(19) United States
(12) Patent Application Publication
(10) Pub. No.: US 2009/0170812 A1
(43) Pub. Date: Jul. 2, 2009

(54) Transcription factor modulating compounds and methods of use thereof

(75) Inventors: Michael N. Alekshun, Marlboro, NJ (US); Victoria Bartlett, Franklin, MA (US); Lynne Garrity-Ryan, Melrose, MA (US); Mark Grier, Medford, MA (US); Oak K. Kim, Cambridge, MA (US); Atul K. Verma, Mansfield, MA (US)

Correspondence Address: MCCARTER & ENGLISH, LLP BOSTON, 265 Franklin Street, Boston, MA, 02110, US

(73) Assignee: Paratek Pharmaceuticals, Inc., Boston, MA (US)

(21) Appl. No.: 11/823,103

(22) Filed: Jun. 25, 2007

Related U.S. Application Data
(60) Provisional application No. 60/815,984, filed on Jun. 23, 2006.

(51) Int. Cl. A61K 31/675; C12N 1/00; C07K 14/00; A61K 31/4184; A61K 31/437; A61K 31/4196; A61K 31/421; A61K 31/5377; A61K 31/496; A61K 31/4439; C07D 235/04; C07D 471/02; C07D 249/08; C07D 263/30; C07D 413/02; C07D 403/02; C07D 401/02; C07F 9/06; A01N 43/52; A01N 43/40; A01N 43/653; A01N 43/76; A01N 43/84; A01N 43/60; A01P 1/00; A61P 31/00; A61P 13/02

(52) U.S. Cl. 514/80; 435/243; 530/350; 514/394; 514/303; 514/383; 514/374; 514/234.5; 514/254.06; 514/338; 548/304.4; 546/118; 548/266.4; 548/215; 544/139; 544/370; 546/273.4; 548/113

(57) ABSTRACT

Substituted benzoimidazole compounds useful as anti-infectives that decrease resistance, virulence, or growth of microbes are provided. Methods of making and using substituted benzoimidazole compounds, as well as pharmaceutical preparations thereof, in, e.g., reducing antibiotic resistance and inhibiting biofilms.

### RELATED APPLICATIONS

This application claims priority to U.S. Provisional Patent Application No. 60/815,984, filed on Jun. 23, 2006. The contents of the aforementioned application are hereby incorporated in their entirety.

### BACKGROUND OF THE INVENTION

Most antibiotics currently used and in development to treat bacterial infections impose selective pressure on microorganisms and have led to the development of widespread antibiotic resistance. Therefore, the development of an alternative approach to treating microbial infections would be of great benefit.

Multidrug resistance in bacteria is generally attributed to the acquisition of multiple transposons and plasmids bearing genetic determinants for different mechanisms of resistance (Gold et al. 1996. N. Engl. J. Med. 335:1445). However, descriptions of intrinsic mechanisms that confer multidrug resistance have begun to emerge. The first of these was a chromosomally encoded multiple antibiotic resistance (mar) locus in Escherichia coli (George and Levy, 1983. J. Bacteriol. 155:531; George and Levy 1983 J. Bacteriol. 155:541). Mar mutants of E. coli arose at a frequency of 10⁻⁶ to 10⁻⁷ and were selected by growth on subinhibitory levels of tetracycline or chloramphenicol (George and Levy, supra). These mutants exhibited resistance to tetracyclines, chloramphenicol, penicillins, cephalosporins, puromycin, nalidixic acid, and rifampin (George and Levy, supra). Later, the resistance phenotype was extended to include fluoroquinolones (Cohen et al. 1989. Antimicrob. Agents Chemother. 33:1318), oxidative stress agents (Ariza et al. 1994. J. Bacteriol. 176:143; Greenberg et al. 1991. J. Bacteriol. 73:4433), and more recently, organic solvents (White et al. 1997. J. of Bacteriology 179:6122; Asako, et al. 1997. J. Bacteriol. 176:143) and household disinfectants, e.g., pine oil and/or TRICLOSAN® (McMurry et al. 1998. FEMS Microbiology Letters 166:305; Moken et al. 1997. Antimicrobial Agents and Chemotherapy 41:2770).

The mar locus consists of two divergently positioned transcriptional units that flank a common promoter/operator region in E. coli, Salmonella typhimurium, and other Entrobacteriacae (Alekshun and Levy. 1997, Antimicrobial Agents and Chemother. 41: 2067). One operon encodes MarC, a putative integral inner membrane protein without any yet apparent function, but which appears to contribute to the Mar phenotype in some strains. The other operon comprises marRAB, encoding the Mar repressor (MarR), which binds marO and negatively regulates expression of marRAB (Cohen et al. 1994. J. Bacteriol. 175:1484; Martin and Rosner 1995. PNAS 92:5456; Seoane and Levy. 1995 J. Bacteriol. 177:530), an activator (MarA), which controls expression of other genes on the chromosome, e.g., the mar regulon (Cohen et al. 1994 J. Bacteriol. 175:1484; Gambino et. al. 1993. J. Bacteriol. 175:2888; Seoane and Levy, 1995 J. Bacteriol. 177:530), and a putative small protein (MarB) of unknown function.

Exposure of E. coli to several chemicals, including tetracycline and chloramphenicol (Hachler et al. 1991 J. Bacteriol 173(17):5532-8; Ariza, 1994, J Bacteriol; 176(1):143-8), sodium salicylate and its derivatives (Cohen, 1993, J Bacteriol; 175(24):7856-62) and oxidative stress agents (Seoane et al. 1995. J Bacteriol; 177(12):3414-9) induces the Mar phenotype. Some of these chemicals act directly at the level of MarR by interacting with the repressor and inactivating its function (Alekshun. 1999. J. Bacteriol. 181:3303-3306) while others (antibiotics such as tetracycline and chloramphenicol) appear to induce mar expression by an alternative mechanism (Alekshun. 1999. J. Bacteriol. 181:3303-3306) e.g., through a signal transduction pathway.

Once expressed, MarA activates the transcription of several genes that constitute the E. coli mar regulon (Alekshun, 1997, Antimicrob. Agents Chemother. 41:2067-2075; Alekshun, 1999, J. Bacteriol. 181:3303-3306). With respect to decreased antibiotic susceptibility, the increased expression of the AcrAB/TolC multidrug efflux system (Fralick, 1996, J. Bacteriol. 178(19):5803-5; Okusu, 1996 J Bacteriol; 178(1):306-8) and decreased synthesis of OmpF (Cohen, 1988, J. Bacteriol.; 170(12):5416-22) an outer membrane protein, play major roles. Organic solvent tolerance, however, is attributed to MarA mediating increased expression of AcrAB, TolC, OmpX, and a 77 kDa protein (Aono, 1998, Extremophiles; 2(3):239-48; Aono, 1998 J Bacteriol; 180(4):938-44.) but is independent of OmpF levels (Asako, 1999, Appl Environ Microbiol; 65(1):294-6).

MarA is a member of the XylS/AraC family of transcriptional activators (Gallegos et al. 1993. Nucleic Acids Res. 21:807). There are more than 100 proteins within the XyIS/AraC family and a defining characteristic of this group of proteins is the presence of two helix-turn-helix (HTH) DNA binding motifs. Proteins within this family activate many different genes, some of which produce antibiotic and oxidative stress resistance or control microbial metabolism and virulence (Gallegos et al. supra).

### SUMMARY OF THE INVENTION

The instant invention identifies microbial transcription factors, e.g., transcription factors of the AraC-XylS family, as virulence factors in microbes and shows that inhibition of these factors reduces the virulence of microbial cells. Because these transcription factors control virulence, rather than essential cellular processes, the development of resistance is much less likely. Accordingly, in one aspect, the invention is directed to a method for preventing infection of a subject by a microbe comprising: administering a compound that modulates the expression or activity of a microbial transcription factor to a subject at risk of developing an infection such that infection of the subject is prevented.

In one embodiment, the invention pertains, at least in part, to a method for reducing antibiotic resistance of a microbial cell. The method includes contacting the cell with a transcription factor modulating compound of the formula (I):

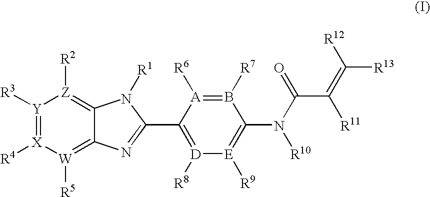

wherein

R¹ is hydroxyl, OCOCO₂H; a straight or branched C₁-C₅ alkyloxy group; or a straight or branched C₁-C₅ alkyl group;

A, B, D, E, W, X, Y and Z are each independently carbon or nitrogen;

wherein R², R³, R⁴, R⁵, R⁶, R⁷, R⁸, R⁹ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen when A, B, D, E, W, X, Y and Z are carbon; or wherein R², R³, R⁴, R⁵, R⁶, R⁷, R⁸, R⁹ are each independently absent or hydroxyl when A, B, D, E, W, X, Y and Z are nitrogen;

R¹⁰, R¹¹, R¹² and R¹³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen; and pharmaceutically acceptable salts, esters and prodrugs thereof;

provided that when A, B, C, D, E, W, X, Y and Z are each carbon, one of R⁶, R⁷, R⁸, R⁹ is not hydrogen, such that the antibiotic resistance of said microbial cell is reduced.

In another embodiment, the invention pertains, at least in part, to a method for modulating transcription, comprising contacting a transcription factor with a transcription factor modulating compound of the formula (I):

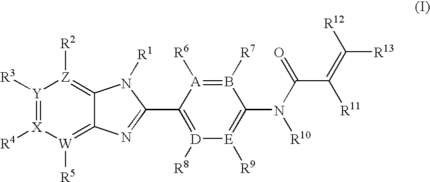

wherein

R¹ is hydroxyl, OCOCO₂H; a straight or branched C₁-C₅ alkyloxy group; or a straight or branched C₁-C₅ alkyl group;

A, B, D, E, W, X, Y and Z are each independently carbon or nitrogen;

wherein R², R³, R⁴, R⁵, R⁶, R⁷, R⁸, R⁹ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen when A, B, D, E, W, X, Y and Z are carbon; or wherein R², R³, R⁴, R⁵, R⁶, R⁷, R⁸, R⁹ are each independently absent or hydroxyl when A, B, D, E, W, X, Y and Z are nitrogen; and

R¹⁰, R¹¹, R¹² and R¹³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen; and pharmaceutically acceptable salts, esters and prodrugs thereof;

provided that when A, B, D, E, W, X, Y and Z are each carbon, one of R⁶, R⁷, R⁸, R⁹ is not hydrogen, such that the transcription is modulated.

In one embodiment, the invention pertains, at least in part, to a method for reducing antibiotic resistance of a microbial cell, comprising contacting said cell with a transcription factor modulating compound of the formula (II):

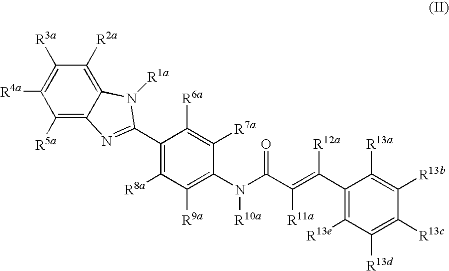

wherein

R^{1a} is hydroxyl, OCOCO₂H, a straight or branched C₁-C₅ alkyloxy group, or a straight or branched C₁-C₅ alkyl group;

R^{2a}, R^{3a}, R^{4a}, R^{5a}, R^{6a}, R^{7a}, R^{8a}, R^{9a}, R^{10a}, R^{11a}, R^{12a}, R^{13a}, R^{13b}, R^{13c}, R^{13d} and R^{13e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof;

provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b}, R^{13d}, and R^{13e} are hydrogen, then R^{13c} is not hydrogen, fluorine, dimethylamino, cyano, hydroxy, methyl or methoxy; and

provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b} and R^{13d} are hydrogen, then R^{13c} and R^{13e} are not fluorine; such that the antibiotic resistance of said microbial cell is reduced.

In yet another embodiment, the invention pertains, at least in part, to a method for modulating transcription, comprising contacting a transcription factor with a transcription factor modulating compound of the formula (II):

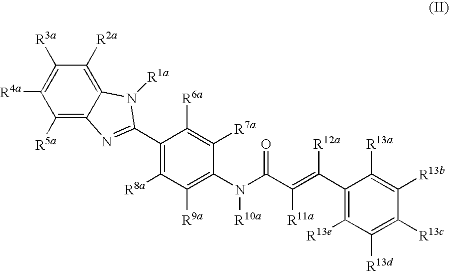

wherein

R^{1a} is hydroxyl, OCOCO₂H, a straight or branched C₁-C₅ alkyloxy group, or a straight or branched C₁-C₅ alkyl group;

R^{2a}, R^{3a}, R^{4a}, R^{5a}, R^{6a}, R^{7a}, R^{8a}, R^{9a}, R^{10a}, R^{11a}, R^{12a}, R^{13a}, R^{13b}, R^{13c}, R^{13d} and R^{13e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof;

provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b}, R^{13d}, and R^{13e} are hydrogen, then R^{13c} is not hydrogen, fluorine, dimethylamino, cyano, hydroxy, methyl or methoxy; and

provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b} and R^{13d} are hydrogen, then R^{13c} and R^{13e} are not fluorine; such that transcription is modulated.

In another embodiment, the invention pertains, at least in part, to a method for reducing antibiotic resistance of a microbial cell, comprising contacting said cell with a transcription factor modulating compound of the formula (III):

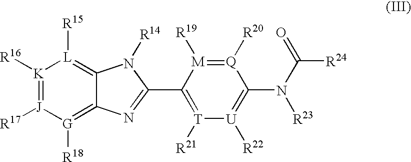

wherein

R¹⁴ is hydroxyl, OCOCO₂H, a straight or branched C₁-C₅ alkyloxy group, or a straight or branched C₁-C₅ alkyl group;

G, J, K, L, M, Q, T and U are each independently carbon or nitrogen;

wherein R¹⁵, R¹⁶, R¹⁷, R¹⁸, R¹⁹, R²⁰, R²¹, R²², R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen when G, J, K, L, M, Q, T and U are carbon; or R¹⁵, R¹⁶, R¹⁷, R¹⁸, R¹⁹, R²⁰, R²¹, R²², R²³ and R²⁴ are each independently absent or hydroxyl when G, J, K, L, M, Q, T and U are nitrogen;

R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen; and pharmaceutically acceptable salts, esters and prodrugs thereof;

provided that when G, J, K, L, M, Q, T and U are each carbon, one of R¹⁵, R¹⁶, R¹⁷, R¹⁸, R¹⁹, R²⁰, R²¹, R²², R²³ and R²⁴, are not hydrogen, such that the antibiotic resistance of said microbial cell is reduced.

In yet another embodiment, the invention pertains, at least in part, to a method for modulating transcription, comprising contacting a transcription factor with a transcription factor modulating compound of the formula (III):

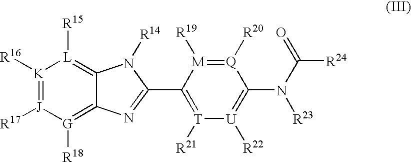

wherein

R¹⁴ is hydroxyl, OCOCO₂H, a straight or branched C₁-C₅ alkyloxy group, or a straight or branched C₁-C₅ alkyl group;

G, J, K, L, M, Q, T and U are each independently carbon or nitrogen;

wherein R¹⁵, R¹⁶, R¹⁷, R¹⁸, R¹⁹, R²⁰, R²¹, R²², R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen when G, J, K, L, M, Q, T and U are carbon; or R¹⁵, R¹⁶, R¹⁷, R¹⁸, R¹⁹, R²⁰, R²¹, R²², R²³ and R²⁴ are each independently absent or hydroxyl when G, J, K, L, M, Q, T and U are nitrogen;

R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen; and pharmaceutically acceptable salts, esters and prodrugs thereof;

provided that when G, J, K, L, M, Q, T and U are each carbon, one of R¹⁵, R¹⁶, R¹⁷, R¹⁸, R¹⁹, R²⁰, R²¹, R²², R²³ and R²⁴, are not hydrogen, such that transcription is modulated.

In one embodiment, the invention pertains, at least in part, to a method for reducing antibiotic resistance of a microbial cell, comprising contacting said cell with a transcription factor modulating compound of the formula (IV):

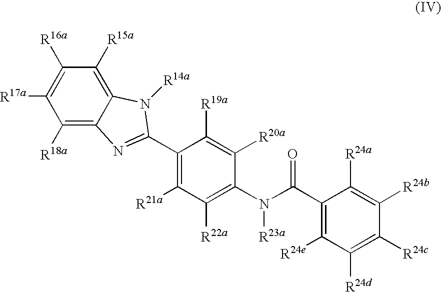

wherein

R^{14a} is hydroxyl, OCOCO₂H, a straight or branched C₁-C₅ alkyloxy group, or a straight or branched C₁-C₅ alkyl group;

R^{15a}, R^{16a}, R^{17a}, R^{18a}, R^{19a}, R^{20a}, R^{21a}, R^{22a}, R^{23a} and R^{24a}, R^{24b}, R^{24c}, R^{24d} and R^{24e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof;

provided that at least two of R^{24a}, R^{24b}, R^{24c}, R^{24d} and R^{24e} are not hydrogen, such that the antibiotic resistance of said microbial cell is reduced.

In another embodiment, the invention pertains, at least in part, to a method for modulating transcription, comprising contacting a transcription factor with a transcription factor modulating compound of the formula (IV):

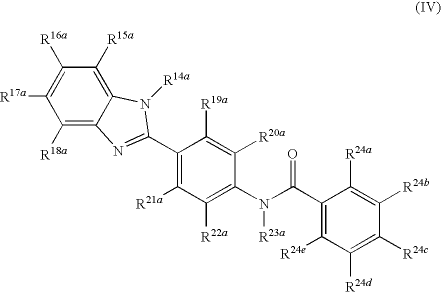

wherein

R^{14a} is hydroxyl, OCOCO₂H, a straight or branched C₁-C₅ alkyloxy group, or a straight or branched C₁-C₅ alkyl group;

R^{15a}, R^{16a}, R^{17a}, R^{18a}, R^{19a}, R^{20a}, R^{21a}, R^{22a}, R^{23a} and R^{24a}, R^{24b}, R^{24c}, R^{24d} and R^{24e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; or R^{24c} and R^{24d} are connected to form a ring; and esters, prodrugs and pharmaceutically acceptable salts thereof;

provided that at least two of R^{24a}, R^{24b}, R^{24c}, R^{24d} and R^{24e} are not hydrogen, such that transcription is modulated.

In a further embodiment, the invention pertains, at least in part, to a method for reducing antibiotic resistance of a microbial cell, comprising contacting said cell with a transcription factor modulating compound of the formula (V):

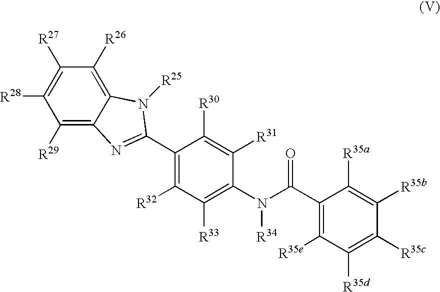

wherein

R²⁵ is hydroxyl, OCOCO₂H, a straight or branched C₁-C₅ alkyloxy group, or a straight or branched C₁-C₅ alkyl group;

R²⁶, R²⁷, R²⁸, R²⁹, R³⁰, R³¹, R³², R³³, R³⁴, R^{35a}, R^{35b}, R^{35c}, R^{35d}, and R^{35e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof;

provided that at least two of R²⁶, R²⁷, R²⁸ and R²⁹ are not hydrogen, such that the antibiotic resistance of said microbial cell is reduced.

In another embodiment, the invention pertains to a method for modulating transcription, comprising contacting a transcription factor with a transcription factor modulating compound of the formula (V):

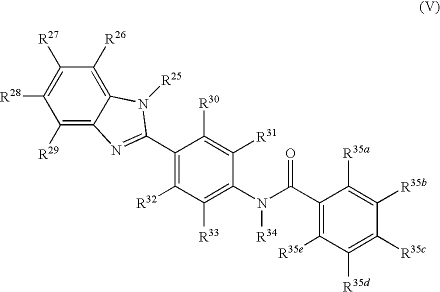

wherein

R²⁵ is hydroxyl, OCOCO₂H, a straight or branched C₁-C₅ alkyloxy group, or a straight or branched C₁-C₅ alkyl group;

R²⁶, R²⁷, R²⁸, R²⁹, R³⁰, R³¹, R³², R³³, R³⁴, R^{35a}, R^{35b}, R^{35c}, R^{35d}, and R^{35e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen; and esters, prodrugs and pharmaceutically acceptable salts thereof;

provided that at least two of R²⁶, R²⁷, R²⁸ and R²⁹ are not hydrogen, such that transcription is modulated.

In one embodiment, the invention pertains to a method for reducing antibiotic resistance of a microbial cell, comprising contacting said cell with a transcription factor modulating compound of the formula (VI):

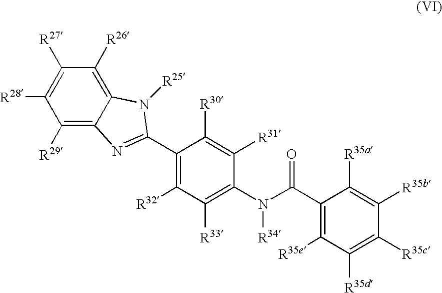

wherein

R^{25′} is a substituted straight or branched C₁-C₅ alkyloxy group;

R^{26′}, R^{27′}, R^{28′}, R^{29′}, R^{30′}, R^{31′}, R^{32′}, R^{33′}, R^{34′}, R^{35a′}, R^{35b′}, R^{35c′}, R^{35d′}, and R^{35e′} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof; such that the antibiotic resistance of said microbial cell is reduced.

In another embodiment, the invention pertains to a method for modulating transcription, comprising contacting a transcription factor with a transcription factor modulating compound of the formula (VI):

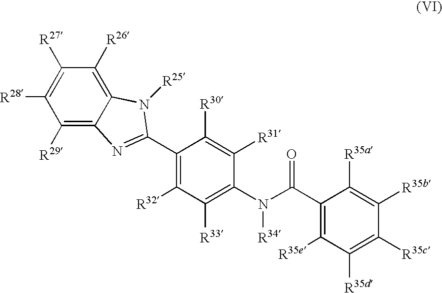

wherein

R^{25′} is substituted straight or branched C₁-C₅ alkoxy group;

R^{26′}, R^{27′}, R^{28′}, R^{29′}, R^{30′}, R^{31′}, R^{32′}, R^{33′}, R^{34′}, R^{35a′}, R^{35b′}, R^{35c′}, R^{35d′}, and R^{35e′} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof; such that transcription is modulated.

In another embodiment, the present invention, pertains, at least in part, to a method for reducing antibiotic resistance of a microbial cell, comprising contacting said cell with a transcription factor modulating compound of the formula (VII):

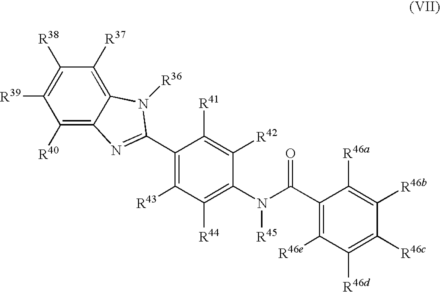

wherein

R³⁶ is hydroxyl;

R³⁷, R³⁹, R⁴⁰, R⁴¹, R⁴², R⁴³, R⁴⁴, R⁴⁵, R^{46a}, R^{46b}, R^{46c}, R^{46d} and R^{46e} are each independently hydrogen, alkyl alkenyl, alkynyl, aryl, heteroaryl, heterocyclyl, alkoxy, aryloxy, heteroaryloxy, alkoxycarbonyl aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;

R³⁸ is cyano, nitro, oxime, alkyloxime, aryloxime, heteroaryl, amino-oxime, or aminocarbonyl;

R^{46c} is hydrogen, acyl, fluoro, pyrizinyl, pyridinyl, cyano, imidazolyl, dialkylaminocarbonyl or dialkylamino; and esters, prodrugs and pharmaceutically acceptable salts thereof,

provided that when R³⁸ is nitro and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino, acyl or hydrogen; and

provided that when R³⁸ is cyano and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino; such that the antibiotic resistance of said microbial cell is reduced.

In a further embodiment, the present invention pertains, at least in part, to a method for modulating transcription, comprising contacting a transcription factor with a transcription factor modulating compound of the formula (VII):

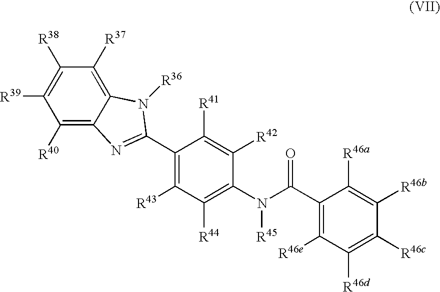

wherein

R³⁶ is hydroxyl;

R³⁷, R³⁹, R⁴⁰, R⁴¹, R⁴², R⁴³, R⁴⁴, R⁴⁵, R^{46a}, R^{46b}, R^{46d}, and R^{46e} are each independently hydrogen, alkyl alkenyl, alkynyl, aryl, heteroaryl, heterocyclyl, alkoxy, aryloxy, heteroaryloxy, alkoxycarbonyl aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;

R³⁸ is cyano, nitro, oxime, alkyloxime, aryloxime, heteroaryl, amino-oxime, or aminocarbonyl;

R^{46c} is hydrogen, acyl, fluoro, pyrizinyl, pyridinyl, cyano, imidazolyl, dialkylaminocarbonyl or dialkylamino; and esters, prodrugs and pharmaceutically acceptable salts thereof;

provided that when R³⁸ is nitro and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino, acyl or hydrogen; and

provided that when R³⁸ is cyano and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino; such that transcription is modulated.

In a further embodiment, the present invention pertains, at least in part, to a method for reducing antibiotic resistance of a microbial cell, comprising contacting said cell with a transcription factor modulating compound of the formula (VIII):

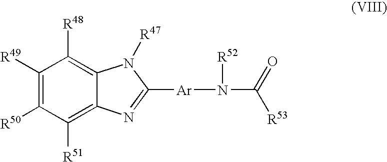

wherein

R⁴⁷ is hydroxyl, OCOCO₂H, a straight or branched C₁-C₅ alkyloxy group, or a straight or branched C₁-C₅ alkyl group;

R⁴⁸, R⁴⁹, R⁵⁰, R⁵¹, R⁵² and R⁵³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;

Ar is aryl; and pharmaceutically acceptable salts, esters and prodrugs thereof; such that the antibiotic resistance of said microbial cell is reduced.

In one embodiment, the present invention pertains, at least in part, to a method for modulating transcription, comprising contacting a transcription factor with a transcription factor modulating compound of the formula (VIII):

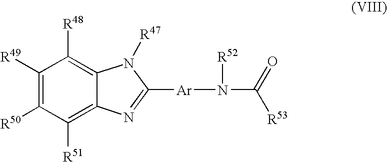

wherein

R⁴⁷ is hydroxyl, OCOCO₂H, a straight or branched C₁-C₅ alkyloxy group, or a straight or branched C₁-C₅ alkyl group;

R⁴⁸, R⁴⁹, R⁵⁰, R⁵¹, R⁵² and R⁵³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;

Ar is aryl; and pharmaceutically acceptable salts, esters and prodrugs thereof; such that transcription is modulated.

In one embodiment, the transcription factor is a member of the AraC-XylS family of transcription factors.

In one embodiment, the transcription factor is a member of the MarA family of transcription factors.

In another embodiment, the method further comprises administering an antibiotic.

In another aspect, the invention pertains to a method for preventing urinary tract infection of a subject by a microbe comprising: administering a compound that modulates the expression or activity of a microbial transcription factor to a subject at risk of developing a urinary tract infection such that infection of the subject is prevented. In one embodiment, the transcription factor is a member of the AraC-XylS family of transcription factors. In one embodiment, the transcription factor is a member of the MarA family of transcription factors. In another embodiment, the method further comprises administering an antibiotic.

In yet another aspect, the invention pertains to a method for reducing virulence of a microbe comprising: administering a compound that modulates the expression or activity of a microbial transcription factor to a subject at risk of developing an infection with the microbe such that virulence of the microbe is reduced.

In another aspect, the invention pertains to a method for treating a microbial infection in a subject comprising: administering a compound that modulates the expression or activity of a transcription factor to a subject having a microbial infection such that infection of the subject is treated. In one embodiment, the transcription factor is a member of the AraC-XylS family of transcription factors. In one embodiment, the transcription factor is a member of the MarA family of transcription factors. In another embodiment, the method further comprises administering an antibiotic.

In another aspect, the invention pertains to a method for evaluating the effectiveness of a compound that modulates the expression or activity of a microbial transcription factor at inhibiting microbial virulence comprising: infecting a non-human animal with a microbe, wherein the ability of the microbe to establish an infection in the non-human animal requires that the microbe colonize the animal; administering the compound that modulates the expression or activity of the microbial transcription factor to the non-human animal; and determining the level of infection of the non-human animal, wherein the ability of the compound to reduce the level of infection of the animal indicates that the compound is effective at inhibiting microbial virulence. In one embodiment, the transcription factor is a member of the AraC-XylS family of transcription factors. In one embodiment, the transcription factor is a member of the MarA family of transcription factors. In another embodiment, the method further comprises administering an antibiotic.

In still another embodiment, the level of infection of the non-human animal is determined by measuring the ability of the microbe to colonize the tissue of the non-human animal.

In another embodiment, the level of infection of the non-human animal is determined by enumerating the number of microbes present in the tissue of the non-human animal.

In another aspect, the invention pertains to a method for identifying a compound for treating microbial infection, comprising: inoculating a non-human animal with a microbe, wherein the ability of the microbe to establish an infection in the non-human animal requires that the microbe colonize the animal; administering a compound which reduces the expression or activity of a microbial transcription factor to the animal, and determining the effect of the test compound on the ability of the microbe to colonize the animal, such that a compound for treating microbial infection is identified. In one embodiment, the transcription factor is a member of the AraC-XylS family of transcription factors. In one embodiment, the transcription factor is a member of the MarA family of transcription factors. In another embodiment, the method further comprises administering an antibiotic.

In still another embodiment, the level of infection of the non-human animal is determined by measuring the ability of the microbe to colonize the tissue of the non-human animal.

In another embodiment, the level of infection of the non-human animal is determined by enumerating the number of microbes present in the tissue of the non-human animal.

In another aspect, method for identifying a compound for reducing microbial virulence, comprising: inoculating a non-human animal with a microbe, wherein the ability of the microbe to establish an infection in the non-human animal requires that the microbe colonize the animal; administering a compound which reduces the expression or activity of a microbial transcription factor to the animal, and determining the effect of the test compound on the ability of the microbe to colonize the animal, such that a compound for reducing microbial virulence is identified. In one embodiment, the transcription factor is a member of the AraC-XylS family of transcription factors. In one embodiment, the transcription factor is a member of the MarA family of transcription factors. In another embodiment, the method further comprises administering an antibiotic.

In yet another embodiment, the level of infection of the non-human animal is determined by measuring the ability of the microbe to colonize the tissue of the non-human animal.

In another embodiment, the level of infection of the non-human animal is determined by enumerating the number of microbes present in the tissue of the non-human animal.

In another aspect, the invention pertains to a method for identifying transcription factors which promote microbial virulence comprising: creating a microbe in which a transcription factor to be tested is misexpressed; introducing the microbe into a non-human animal; wherein the ability of the microbe to establish an infection in the non-human animal requires that the microbe colonize the animal; and determining the ability of the microbe to colonize the animal, wherein a reduced ability of the microbe to colonize the animal as compared to a wild-type microbial cell identifies the transcription factor as a transcription factor which promotes microbial virulence. In another embodiment, the transcription factor is a member of the AraC-XylS family of transcription factors. In another embodiment, the transcription factor is a member of the MarA family of transcription factors.

In another embodiment, the level of infection of the non-human animal is determined by measuring the ability of the microbe to colonize the tissue of the non-human animal.

In another embodiment, the level of infection of the non-human animal is determined by enumerating the number of microbes present in the tissue of the non-human animal.

In another aspect, the invention pertains to a method for reducing the ability of a microbe to adhere to an abiotic surface comprising: contacting the abiotic surface or the microbe with a compound that modulates the activity of a transcription factor such that the ability of the microbe to adhere to the abiotic surface is reduced. In one embodiment, the transcription factor is a member of the AraC-XylS family of transcription factors. In another embodiment, the transcription factor is a member of the MarA family of transcription factors.

In yet another embodiment, the method further comprises contacting the abiotic surface or the microbe with a second agent that is effective at controlling the growth of the microbe.

In still another embodiment, the abiotic surface is selected from the group consisting of: stents, catheters, and prosthetic devices.

In one aspect, the invention pertains to a pharmaceutical composition comprising a compound that modulates the activity or expression of a microbial transcription factor and a pharmaceutically acceptable carrier, wherein the compound reduces microbial virulence.

In another aspect, the invention pertains to a pharmaceutical composition comprising a compound that modulates the activity or expression of a microbial transcription factor and an antibiotic in a pharmaceutically acceptable carrier.

The present invention represents an advance over the prior art by identifying transcription factor modulating compounds, such as, but not limited to helix-turn-helix protein modulating compounds, and providing novel assays that can be used to identify compounds which modulate microbial transcription factors, such as MarA family polypeptides and AraC family polypeptides. Modulation of gene transcription brought about by the modulation of transcription factors, such as helix-turn-helix domain containing proteins, can control a wide variety of cellular processes. For example, in prokaryotic cells processes such as metabolism, resistance, and virulence can be controlled.

Assays to identify compounds that are capable of modulating bacterial transcription factors would be of great benefit in the identification of agonists and antagonists that can be used to control gene transcription in both prokaryotic and eukaryotic cells.

In one embodiment, the invention pertains to a method for reducing antibiotic resistance of a cell, e.g., a eukaryotic or prokaryotic cell. In a preferred embodiment, the cell is a microbial cell. In one embodiment, the invention pertains to a method for reducing antibiotic resistance in a microbial cell, by contacting a cell with a transcription factor modulating compound, such that the antibiotic resistance of the cell is reduced.

In another embodiment, the invention also includes methods for identifying transcription factor modulating compounds. The method includes contacting a microbial cell with a test compound under conditions which allow interaction of the compound with the microbial cell and measuring the ability of the test compound to affect the cell. The microbial cell includes a selective marker under the direct control of a transcription factor responsive element and a transcription factor.

In yet another embodiment, the invention includes methods for identifying a transcription factor modulating compound. The method includes contacting a microbial cell comprising: 1) a selective marker under the control of a transcription factor responsive element and 2) a transcription factor, with a test compound under conditions which allow interaction of the compound with the microbial cell, and measuring the ability of the test compound to affect the growth (e.g., in vitro or in vivo) or survival of the microbial cell, wherein the inactivation of the transcription factor leads to a decrease in in vitro or in vivo cell survival. The invention also pertains to similar methods where the inactivation of the transcription factor leads to an increase in cell survival, as well as methods wherein the activation of the transcription factor leads to increased or, alternatively, decreased cell survival.

In another embodiment, the invention also pertains to methods for identifying a transcription factor modulating compound, by contacting a microbial cell comprising: 1) a chromosomal deletion in a guaB or purA gene, 2) heterologous guaB or purA gene under the control of its transcription factor responsive promoter, and 3) a transcription factor, with a test compound under conditions which allow interaction of the compound with the microbial cell. The method further includes the steps of measuring the ability of the compound to affect gene expression of the reporter or the growth or survival of the microbial cell as an indication of whether the compound modulates the activity of a transcription factor. The ability of the compound to modulate the activity of a transcription factor leads to an alteration in gene expression may effect cell growth or survival.

The invention pertains to transcription factor modulating compounds, HTH protein modulating compounds, and MarA family modulating compounds identified by the methods of the invention, methods of using these compounds and pharmaceutical compositions comprising these compounds. The transcription factor modulating compounds of the invention include, but are not limited to, compounds of formulae (I)-(VIII) and Table 2.

The invention also pertains, at least in part, to a kit for identifying a transcription factor modulating compound which modulates the activity of a transcription factor polypeptide comprising a microbial cell. The kit includes 1) a selective marker under the control of a transcription factor responsive element and 2) a transcription factor.

The invention also pertains, at least in part, to pharmaceutical compositions which contain an effective amount of a transcription factor modulating compound, and, optionally, a pharmaceutically acceptable carrier.

The invention also pertains to a method of inhibiting a biofilm, by administering a composition comprising a transcription factor modulating compound such that the biofilm is inhibited.

In another embodiment, the invention pertains to a pharmaceutical composition comprising a pharmaceutically acceptable carrier and a transcription factor modulating compound. The transcription factor modulating compound may be of the formulae (I)-(VIII) and Table 2.

### DETAILED DESCRIPTION OF THE INVENTION

The instant invention identifies microbial transcription factors, e.g., transcription factors of the AraC-XylS family, as virulence factors in microbes and shows that inhibition of these factors reduces the virulence of microbial cells. Because these transcription factors control virulence, rather than essential cellular processes, modulation of these factors should not promote resistance.

Some major families of transcription factors found in bacteria include the helix-turn-helix transcription factors (HTH) (Harrison, S. C., and A. K. Aggarwal 1990. Annual Review of Biochemistry. 59:933-969) such as AraC, MarA, Rob, SoxS and LysR; winged helix transcription factors (Gajiwala, K. S., and S. K. Burley 2000. 10:110-116), e.g., MarR, Sar/Rot family, and OmpR (Huffman, J. L., and R. G. Brennan 2002. Curr Opin Struct Biol. 12:98-106, Martinez-Hackert, E., and A. M. Stock 1997. Structure. 5:109-124); and looped-hinge helix transcription factors (Huffman, J. L., and R. G. Brennan 2002 Curr Opin Struct Biol. 12:98-106), e.g. the AbrB protein family.

The AraC-XylS family of transcription factors comprises many members. MarA, SoxS, Rma, and Rob are examples of proteins within the AraC-XylS family of transcription factors. These factors belong to a subset of the AraC-XylS family that have historically been considered to play roles in promoting resistance to multiple antibiotics and have not been considered to be virulence factors. In fact, the role of marA in virulence has been tested using a marA null mutant of Salmonella enterica serovar Typhimurium (S. typhimurium) in a mouse infection model (Sulavik et al. 1997. J. Bacteriology 179:1857) and no such role has been found. In another model (using co-infection experiments or crude statistics) only a weak effect of a marA null mutant in chickens has been demonstrated (Randall et al. 2001. J. Med. Microbiol. 50:770). In contrast to this earlier work, this invention is based, at least in part, on the finding that the ability of microbes to cause infection in a host can be inhibited by inhibiting the expression and/or activity of microbial transcription factors. Thus, the instant invention validates the use of microbial transcription factors as therapeutic targets.

The invention pertains, at least in part, to compounds which modulate transcription factors (e.g., helix-turn-helix (HTH) proteins, AraC family polypeptides, MarA family polypeptides, etc.), methods of identifying the transcription factor modulating compounds (e.g., HTH protein modulating compounds, AraC family polypeptide modulating compounds, MarA family polypeptide modulating compounds, etc.), and methods of using the compounds.

### I. Transcription Factors

The term “transcription factor” includes proteins that are involved in gene regulation in both prokaryotic and eukaryotic organisms. In one embodiment, transcription factors can have a positive effect on gene expression and, thus, may be referred to as an “activator” or a “transcriptional activation factor.” In another embodiment, a transcription factor can negatively effect gene expression and, thus, may be referred to as “repressors” or a “transcription repression factor.” Activators and repressors are generally used terms and their functions are discerned by those skilled in the art.

As used herein, the term “infectivity” or “virulence” includes the ability of a pathogenic microbe to colonize a host, a first step required in order to establish growth in a host. Infectivity or virulence is required for a microbe to be a pathogen. In addition, a virulent microbe is one which can cause a severe infection. As used herein, the term “pathogen” includes both obligate and opportunistic organisms. The ability of a microbe to resist antibiotics is also important in promoting growth in a host, however, in one embodiment, antibiotic resistance is not included in the terms “infectivity” or “virulence” as used herein. Accordingly, in one embodiment, the instant invention pertains to methods of reducing the infectivity or virulence of a microbe without affecting (e.g., increasing or decreasing) antibiotic resistance. Preferably, as used herein, the term “infectivity or virulence” includes the ability of an organism to establish itself in a host by evading the host's barriers and immunologic defenses.

The term “AraC family polypeptide,” “AraC-XylS family polypeptide” or “AraC-XylS family peptide” include an art recognized group of prokaryotic transcription factors which contains more than 100 different proteins (Gallegos et al., (1997) Micro. Mol. Biol. Rev. 61: 393; Martin and Rosner, (2001) Curr. Opin. Microbiol. 4:132). AraC family polypeptides include proteins defined in the PROSITE (PS) database as profile PS01124. The AraC family polypeptides also include polypeptides described in PS0041, HTH AraC Family 1, and PS01124, and HTH AraC Family 2. Multiple sequence alignments for the AraC-XylS family polypeptides, HTH AraC family 1, and HTH AraC family 2 are shown in FIGS. 1-3, respectively. In an embodiment, the AraC family polypeptides are generally comprised of, at the level of primary sequence, by a conserved stretch of about 100 amino acids, which are believed to be responsible for the DNA binding activity of this protein (Gallegos et al., (1997) Micro. Mol. Biol. Rev. 61: 393; Martin and Rosner, (2001) Curr. Opin. Microbiol. 4: 132). AraC family polypeptides also may include two helix turn helix DNA binding motifs (Martin and Rosner, (2001) Curr. Opin. Microbiol. 4: 132; Gallegos et al., (1997) Micro. Mol. Biol. Rev. 61: 393; Kwon et al., (2000) Nat. Struct. Biol. 7: 424; Rhee et al., (1998) Proc. Natl. Acad. Sci. U.S.A. 95: 10413). The term includes MarA family polypeptides and HTH proteins. In one embodiment, the invention pertains to a method for modulating an AraC family polypeptide, by contacting the AraC family polypeptide with a test compound which interacts with a portion of the polypeptide involved in DNA binding. In a further embodiment, the test compound interacts with a conserved amino acid residue (capitalized) of the HTH AraC family 1 protein indicated in FIG. 2.

The term “helix-turn-helix protein,” “HTH protein,” “helix-turn-helix polypeptides,” and “HTH polypeptides,” includes proteins comprising one or more helix-turn-helix domains. Helix-turn-helix domains are known in the art and have been implicated in DNA binding (Ann Rev. of Biochem. 1984. 53:293). An example of the consensus sequence for a helix-turn domain can be found in Brunelle and Schleif (1989. J. Mol. Biol. 209:607). The domain has been illustrated by the sequence XXXPhoAlaXXPhoGlyPhoXXXXPhoXXPhoXX, where X is any amino acid and Pho is a hydrophobic amino acid.

The helix-turn-helix domain was the first DNA-binding protein motif to be recognized. Although originally the HTH domain was identified in bacterial proteins, the HTH domain has since been found in hundreds of DNA-binding proteins from both eukaryotes and prokaryotes. It is constructed from two alpha helices connected by a short extended chain of amino acids, which constitutes the “turn.”

In one embodiment, a helix-turn-helix domain containing protein is a Mar A family polypeptide. The language “MarA family polypeptide” includes the many naturally occurring HTH proteins, such as transcription regulation proteins which have sequence similarities to MarA and which contain the MarA family signature pattern, which can also be referred to as an XylS/AraC signature pattern. An exemplary signature pattern which defines MarA family polypeptides is shown, e.g., on PROSITE and is represented by the sequence: [KRQ]-[LIVMA]-X(2)-[GSTALIV]-{FYWPGDN}X(2)-[LIVMSA]-X(4,9)-[LIVMF]-X(2)-[LIVMSTA]-X(2)-[GSTACIL]-X(3)-[GANQRF]-[LIVMFY]-X(4,5)-[LFY]-X(3)-[FYIVA]-{FYWHCM}-X(3)-[GSADENQKR]-X-[NSTAPKL]-[PARL], where X is any amino acid. MarA family polypeptides have two “helix-turn-helix” domains. This signature pattern was derived from the region that follows the first, most amino terminal, helix-turn-helix domain (HTH1) and includes the totality of the second, most carboxy terminal helix-turn-helix domain (HTH2). (See PROSITE PS00041).

The MarA family of proteins (“MarA family polypeptides”) represent one subset of AraC-XylS family polypeptides and include proteins like MarA, SoxS, Rob, Rma, AarP, PqrA, etc. The MarA family polypeptides, generally, are involved in regulating resistance to antibiotics, organic solvents, and oxidative stress agents (Alekshun and Levy, (1997) Antimicrob. Agents. Chemother. 41: 2067). Like other AraC-XylS family polypeptides, MarA-like proteins also generally contain two HTH motifs as exemplified by the MarA and Rob crystal structures (Kwon et al., (2000) Nat. Struct. Biol. 7: 424; Rhee et al., (1998) Proc. Natl. Acad. Sci. U.S.A. 95: 10413). Members of the MarA family can be identified by those skilled in the art and will generally be represented by proteins with homology to amino acids 30-76 and 77-106 of MarA (SEQ ID. NO. 1).

Preferably, a MarA family polypeptide or portion thereof comprises the first MarA family HTH domain (HTH1) (Brunelle, 1989, J Mol Biol; 209(4):607-22). In another embodiment, a MarA polypeptide comprises the second MarA family HTH domain (HTH2) (Caswell, 1992, Biochem J; 287:493-509.). In a preferred embodiment, a MarA polypeptide comprises both the first and second MarA family HTH domains.

MarA family polypeptide sequences are “structurally related” to one or more known MarA family members, preferably to MarA. This relatedness can be shown by sequence or structural similarity between two MarA family polypeptide sequences or between two MarA family nucleotide sequences that specify such polypeptides. Sequence similarity can be shown, e.g., by optimally aligning MarA family member sequences using an alignment program for purposes of comparison and comparing corresponding positions. To determine the degree of similarity between sequences, they will be aligned for optimal comparison purposes (e.g., gaps may be introduced in the sequence of one protein for nucleic acid molecule for optimal alignment with the other protein or nucleic acid molecules). The amino acid residues or bases and corresponding amino acid positions or bases are then compared. When a position in one sequence is occupied by the same amino acid residue or by the same base as the corresponding position in the other sequence, then the molecules are identical at that position. If amino acid residues are not identical, they may be similar. As used herein, an amino acid residue is “similar” to another amino acid residue if the two amino acid residues are members of the same family of residues having similar side chains. Families of amino acid residues having similar side chains have been defined in the art (see, for example, Altschul et al. 1990. J. Mol. Biol. 215:403) including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan). The degree (percentage) of similarity between sequences, therefore, is a function of the number of identical or similar positions shared by two sequences (i.e., % homology=# of identical or similar positions/total # of positions×100). Alignment strategies are well known in the art; see, for example, Altschul et al. supra for optimal sequence alignment.

MarA family polypeptides may share some amino acid sequence similarity with MarA. The nucleic acid and amino acid sequences of MarA as well as other MarA family polypeptides are available in the art. For example, the nucleic acid and amino acid sequence of MarA can be found, e.g., on GeneBank (accession number M96235 or in Cohen et al. 1993. J. Bacteriol. 175:1484, or in SEQ ID NO:1 and SEQ ID NO:2.

The nucleic acid and/or amino acid sequences of MarA can be used as “query sequences” to perform a search against databases (e.g., either public or private) to, for example, identify other MarA family members having related sequences. Such searches can be performed, e.g., using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to MarA family nucleic acid molecules. BLAST protein searches can be performed with the XBLAST program, score=50, wordlength=3 to obtain amino acid sequences homologous to MarA protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

MarA family members can also be identified as being similar based on their ability to specifically hybridize to nucleic acid sequences specifying MarA. Such stringent conditions are known to those skilled in the art and can be found (e.g., in Current Protocols in Molecular Biology, John Wiley & Sons, N.Y. (1989), 6.3.1-6.3.6). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Conditions for hybridizations are largely dependent on the melting temperature Tm that is observed for half of the molecules of a substantially pure population of a double-stranded nucleic acid. Tm is the temperature in ° C. at which half the molecules of a given sequence are melted or single-stranded. For nucleic acids of sequence 11 to 23 bases, the Tm can be estimated in degrees C. as 2(number of A+T residues)+4(number of C+G residues). Hybridization or annealing of nucleic acid molecules should be conducted at a temperature lower than the Tm, e.g., 15° C., 20° C., 25° C. or 30° C. lower than the Tm. The effect of salt concentration (in M of NaCl) can also be calculated, see for example, Brown, A., “Hybridization” pp. 503-506, in The Encyclopedia of Molec. Biol., J. Kendrew, Ed., Blackwell, Oxford (1994).

Preferably, the nucleic acid sequence of a MarA family member identified in this way is at least about 10%, 20%, more preferably at least about 30%, more preferably at least about 40% identical and preferably at least about 50%, or 60% identical to a MarA nucleotide sequence. In preferred embodiments, the nucleic acid sequence of a MarA family member is at least about 70%, 80%, preferably at least about 90%, more preferably at least about 95% identical with a MarA nucleotide sequence. Preferably, MarA family members have an amino acid sequence at least about 20%, preferably at least about 30%, more preferably at least about 40% identical and preferably at least about 50%, or 60% or more identical with a MarA amino acid sequence. In preferred embodiments, the nucleic acid sequence of a MarA family member is at least about 70%, 80%, more preferably at least about 90%, or more preferably at least about 95% identical with a MarA nucleotide sequence. However, it will be understood that the level of sequence similarity among microbial regulators of gene transcription, even though members of the same family, is not necessarily high. This is particularly true in the case of divergent genomes where the level of sequence identity may be low, e.g., less than 20% (e.g., B. burgdorferi as compared e.g., to B. subtilis). Accordingly, structural similarity among MarA family members can also be determined based on “three-dimensional correspondence” of amino acid residues. As used herein, the language “three-dimensional correspondence” is meant to includes residues which spatially correspond, e.g., are in the same position of a MarA family polypeptide member as determined, e.g., by x-ray crystallography, but which may not correspond when aligned using a linear alignment program. The language “three-dimensional correspondence” also includes residues which perform the same function, e.g., bind to DNA or bind the same cofactor, as determined, e.g., by mutational analysis.

Exemplary MarA family polypeptides are shown in Table 1, and at Prosite (PS00041) and include: AarP, Ada, AdaA, AdiY, AfrR, AggR, AppY, AraC, CatR, CelD, CfaD, CsvR, D90812, EnvY, ExsA, FapR, HrpB, InF, InvF, LcrF, LumQ, MarA, MelR, MixE, MmsR, MsmR, OrfR, Orf_f375, PchR, PerA, PocR, PqrA, RafR, RamA, RhaR, RhaS, Rns, Rob, SoxS, S52856, TetD, TcpN, ThcR, TmbS, U73857, U34257, U21191, UreR, VirF, XyIR, XylS, Xys1, 2, 3, 4, Ya52, YbbB, YfiF, YisR, YzbC, and YijO. The nucleotide and amino acid sequences of the E. coli, Rob molecule are shown in SEQ ID NO:3 and 4, respectively.

TABLE 1
Some Bacterial MarA homologs^{a}

| Gram-negative | | |
|---|---|---|
| bacteria | | Gram-positive bacteria |
| Escherichia coli | Kiebsiella pneumoniae | Lactobacillus helveticus |
| MarA (1) | RamA (27) | U34257 (38) |
| OrfR (2, 3) | Haemophilus influenzae | Azorhizobium caulinodans |
| SoxS (4, 5) | Ya52 (28) | S52856 (39) |
| AfrR (6) | Yersinia spp. | Streptomyces spp. |
| AraC (7) | CafR (29) | U21191 (40) |
| CelD (8) | LcrF (30) or VirF (30) | AraL (41) |
| D90812 (9) | Providencia stuartii | Streptococcus mutans |
| FapR (10, 11) | AarP (31) | MsmR (42) |
| MelR (12) | Pseudomonas spp. | Pediococcus pentosaceus |
| ORF f375 | MmsR (32) | RafR (43) |
| (13, 14) | TmbS (33) | Photobacterium leiognathi |
| RhaR (15, | XylS (34) | LumQ (44) |
| 16, 17) | Xys1, 2, 3, 4 (35, 36) | Bacillus subtilis |
| RhaS (18) | Cyanobacteria | AdaA (45) |
| Rob (19) | Synechocystis spp. | YbbB (46) |
| U73857 (20) | LumQ (37) | YfiF (47) |
| XylR(21) | PchR (37) | YisR (48) |
| YijO (22) | | YzbC (49) |
| Proteus | | |
| vulgaris | | |
| PqrA (23) | | |
| Salmonella | | |
| typhimurium | | |
| MarA (24) | | |
| InvF (25) | | |
| PocR (26) | | |

^{a}The smaller MarA homologs, ranging in size from 87 (U34257) to 138 (OrfR) amino acid residues, are represented in boldface. References are given in parentheses and are listed below.
References for Table 1:
(1) S. P. Cohen, et al. 1993. J. Bacteriol. 175: 1484-1492
(2) G. M. Braus, et al. 1984. J. Bacteriol. 160: 504-509
(3) K. Schollmeier, et al., 1984. J. Bacteriol 160: 499-503
(4) C. F. Amabile-Cuevas, et al., 1991. Nucleic Acids Res. 19: 4479-4484
(5) J. Wu, et al., 1991. J. Bacteriol. 173: 2864-2871
(6) M. K. Wolf, et al., 1990. Infect. Immun. 58: 1124-1128
(7) C. M. Stoner, et al. 1982. J. Mol. Biol 153: 649-652
(8) L. L. Parker, et al., 1990. Genetics 123: 455-471
(9) H. Mori, 1996. Unpublished data taken from the NCBI databases
(10) P. Klaasen, et al., 1990. Mol. Microbiol. 4: 1779-1783
(11) M. Ahmed, et al., 1994. J. Biol. Chem 269-28506-28513
(12) C. Webster, et al., 1989. Gene 83: 207-213
(13) G. Plunkett, III. 1995. Unpublished
(14) C Garcia-Martin, et al., 1992. J. Gen. Microbiol. 138: 1109-1116
(15) G. Plunkett, III., et al. 1993. Nucleic Acids Res. 21: 3391-3398
(16) C. G. Tate, et al. 1992. J. Biol. Chem. 267: 6923-6932
(17) J. F. Tobin et al., 1987. J. Mol. Biol. 196: 789-799
(18) J. Nishitani, 1991. Gene 105: 37-42
(19) R. E. Benz, et al., 1993. Zentralbl. Bakteriol. Parasitenkd. Infektionskr. Hyg. Abt. 1 Orig. 278: 187-196
(20) M. Duncan, et al., 1996. Unpublished data
(21) H. J. Sofia, et al., 1994. Nucleic Acids Res. 22: 2576-2586
(22) F. R. Blattner, et al., 1993. Nucleic Acids Res. 21: 5408-5417
(23) H. Ishida, et al., 1995. Antimicrob. Agents Chemother. 39: 453-457
(24) M. C. Sulavik, et al., 1997. J. Bacterial. 179: 1857-1866
(25) K. Kaniga, et al., 1994. Mol. Microbiol. 13: 555-568
(26) J. R. Roth, et al. 1993. J. Bacteriol. 175: 3303-3316
(27) A. M. George, et al., 1983. J. Bacteriol. 155: 541-548
(28) R. D. Fleischmann, et al., 1995. Science 269: 469-512
(29) E. E. Galyov, et al, 1991. FEBS Lett. 286: 79-82
(30) N. P. Hoe, et al., 1992. J. Bacteriol. 174: 4275-4286
(31) G. Cornells, et al., 1989. J. Bacteriol. 171: 254-262
(32) D. R. Macinga, et al., 1995. J. Bacteriol. 177: 3407-3413
(33) M. I. Steele, et al., 1992. J. Biol. Chem. 267: 13585-13592
(34) G. Deho, et al., 1995. Unpublished data
(35) N. Mermod, et al., 1984. EMBO J. 3: 2461-2466
(36) S. J. Assinder, et al., 1992. Nucleic Acids Res. 20: 5476
(37) S. J. Assinder, et al., 1993. J. Gen. Microbiol 139: 557-568
(38) E. G. Dudley, et al., 1996. J. Bacteriol. 178: 701-704
(39) D. Geelen, et al., 1995. Unpublished data
(40) J. Kormanec, et al., 1995. Gene 165: 77-80
(41) C. W. Chen, et al., 1992. J. Bacteriol 174: 7762-7769
(42) R. R. Russell, et al., 1992. J. Biol Chem, 267: 4631-4637
(43) K. K. Leenhouts, et al., 1995. Unpublished data
(44) J. W. Lin, et al, 1995. Biochem. Biophys. Res. Commun. 217: 684-695
(45) F. Morohoshi, et al. 1990. Nucleic Acids Res. 18: 5473-5480
(46) M. Rosenberg, et al., 1979. Annu. Rev. Genet. 13: 319-353
(47) H. Yamamoto, et al., 1996. Microbiology 142: 1417-1421
(48) L. B. Bussey, et al., 1993. J. Bacteriol. 175: 6348-6353
(49) P. G. Quirk, et al., 1994. Biochim. Biophys. Acta 1186: 27-34

The term “transcription factor modulating compound” or “transcription factor modulator” includes HTH protein modulating compounds, HTH protein modulators. Transcription factor modulating compounds include compounds which interact with one or more transcription factors, such that the activity of the transcription factor is modulated, e.g., enhanced or inhibited. The term also includes both AraC family modulating compounds and MarA family modulating compounds. In one embodiment, the transcription factor modulating compound is an inhibiting compound of a transcription factor, e.g., a prokaryotic transcription factor or a eukaryotic transcription activation factor. In one embodiment, the transcription factor modulating compounds modulate the activity of a transcription factor as measured by assays known in the art or LANCE assays such as those described in Example 8 of U.S. Ser. No. 11/115,024, incorporated herein by reference. In one embodiment, the transcription factor modulating compound inhibits a particular transcription factor by about 10% or greater, about 40% or greater, about 50% or greater, about 60% or greater, about 70% or greater, about 80% or greater, about 90% or greater, about 95% or greater, or about 100% as compared to the activity of the transcription factor with out the transcription factor modulating compound. In another embodiment, the transcription factor modulating compound inhibits biofilm formation. In one embodiment, the transcription factor modulating compound inhibits biofilm formation as measured by assays known in the art or the Crystal Violet assay described in Example 7 of U.S. Ser. No. 11/115,024, incorporated herein by reference. In one embodiment, the transcription factor of the invention inhibits the formation of a biofilm by about 25% or more, 50% or more, 75% or more, 80% or more, 90% or more, 95% or more, 96% or more, 97% or more, 98% or more, 99% or more, 99.9% or more, 99.99% or more, or by 100%, as compared to the formation of a biofilm without the transcription factor modulating compound.

The term “HTH protein modulating compound” or “HTH protein modulator” includes compounds which interact with one or more HTH proteins such that the activity of the HTH protein is modulated, e.g., enhanced or, inhibited. In one embodiment, the HTH protein modulating compound is a MarA family polypeptide modulating compound. In one embodiment, the activity of the HTH protein is enhanced when it interacts with the HTH protein modulating compound. For example, the activity of the HTH protein may be increased by greater than 10%, greater the 20%, greater than 50%, greater than 75%, greater than 80%, greater than 90%, or 100% of the activity of the HTH protein in the absence of the HTH modulating compound. In another embodiment, the activity of the HTH protein is decreased upon an interaction with the HTH protein modulating compound. In an embodiment, the activity of the HTH protein is decreased by about 25% or more, 50% or more, 75% or more, 80% or more, 90% or more, 95% or more, 96% or more, 97% or more, 98% or more, 99% or more, 99.9% or more, 99.99% or more, or by 100%, as compared to the activity of the protein of a HTH protein when not contacted with an HTH modulating compound of the invention using techniques and assays described herein. Values and ranges included and/or intermediate of the values set forth herein are also intended to be within the scope of the present invention.

The term “MarA family polypeptide modulating compound” or “MarA family modulating compound” include compounds which interact with one or more MarA family polypeptides such that the activity of the MarA family peptide is enhanced or inhibited. In an embodiment, the MarA family polypeptide modulating compound is an inhibiting compound. In a further embodiment, the MarA family inhibiting compound is an inhibitor of MarA, Rob, and/or SoxS. In another embodiment, the MarA family polypeptide modulating compound modulates the expression of luciferase in the Luciferase Assay described in Example 9 of U.S. Ser. No. 11/115,024, incorporated herein by reference. In one embodiment, the MarA family polypeptide modulating compound decreases luciferase expression by greater than 10%, greater than 20%, greater than 30%, greater than 40%, greater than 50%, greater than 60%, greater than 70%, greater than 80%, greater than 90% or about 100%.

The term “polypeptide(s)” refers to a peptide or protein comprising two or more amino acids joined to each other by peptide bonds or modified peptide bonds. “Polypeptide(s)” includes both short chains, commonly referred to as peptides, oligopeptides and oligomers and longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins-Structure And Molecular Properties, 2 Ed., T. E. Creighton, W. H. Freeman and Company, New York (1993) and Wold, F., Posttranslational Protein Modifications: Perspectives and Prospects, pgs. 1-12 in Posttranslational Covalent Modification Of Proteins, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

As used herein, the term “winged helix” includes dimeric transcription factors in which each monomer comprises a helix-turn-helix motif followed by one or two (β-hairpin wings (Brennan. 1993. Cell. 74:773; Gajiwala and Burley. 2000. Curr. Opin. Struct. Biol. 10:110). The classic winged helix motif comprises two wings, three α helices, and three β strands in the sequence H1-B1-H2-T-H3-B2-W1-B3-W2 (where H is a helix, B is a β strand, T is a turn, and W is a wing), although some variation in structure has been demonstrated (Huffman and Brennan. 2002. Current Opinion in Structural Biology. 12:98).

As used herein the term “looped-hinge helix” included transcription factors, such as AbrB which, in the absence of DNA, have revealed a dimeric N-terminal region consisting of a four-stranded β sheet and a C-terminal DNA-binding region comprising one α helix and a “looped hinge” (see, e.g., Huffman and Brennan. 2002 Current Opinion in Structural Biology 12:98). Residues corresponding to R23 and R24 of AbrB are critical for DNA recognition and contribute to the electropositive nature of the DNA-binding region.

Preferred polypeptides (and the nucleic acid molecules that encode them) are “naturally occurring.” As used herein, a ‘naturally-occurring’ molecule refers to a molecule having an amino acid or a nucleotide sequence that occurs in nature (e.g., a natural polypeptide). In addition, naturally or non-naturally occurring variants of the polypeptides and nucleic acid molecules which retain the same functional activity, (such as, the ability to bind to target nucleic acid molecules (e.g., comprising a marbox) or to polypeptides (e.g. RNA polymerase) with a naturally occurring polypeptide are provided for. Such immunologic cross-reactivity can be demonstrated, e.g., by the ability of a variant to bind to a MarA family polypeptide responsive element. Such variants can be made, e.g., by mutation using techniques that are known in the art. Alternatively, variants can be chemically synthesized.

As used herein the term “variant(s)” includes nucleic acid molecules or polypeptides that differ in sequence from a reference nucleic acid molecule or polypeptide, but retain its essential properties. Changes in the nucleotide sequence of the variant may, or may not, alter the amino acid sequence of a polypeptide encoded by the reference nucleic acid molecule. Nucleotide or amino acid changes may result in amino acid substitutions, additions, deletions, fusions and truncations in the polypeptide encoded by a naturally occurring reference sequence. A typical variant of a polypeptide differs in amino acid sequence from a reference polypeptide. Generally, differences are limited so that the sequences of the reference polypeptide and the variant are closely similar overall and, in many regions, identical. A variant and reference polypeptide may differ in amino acid sequence by one or more substitutions, additions, and/or deletions in any combination.

A variant of a nucleic acid molecule or polypeptide may be naturally occurring, such as an allelic variant, or it may be a variant that is not known to occur naturally. Non-naturally occurring variants of nucleic acid molecules and polypeptides may be made from a reference nucleic acid molecule or polypeptide by mutagenesis techniques, by direct synthesis, and by other recombinant methods known to skilled artisans. Alternatively, variants can be chemically synthesized. For instance, artificial or mutant forms of autologous polypeptides which are functionally equivalent, (e.g., have the ability to interact with a MarA family polypeptide responsive element) can be made using techniques which are well known in the art.

Mutations can include, e.g., at least one discrete point mutation which can give rise to a substitution, or by at least one deletion or insertion. For example, mutations can also be made by random mutagenesis or using cassette mutagenesis. For the former, the entire coding region of a molecule is mutagenized by one of several methods (chemical, PCR, doped oligonucleotide synthesis) and that collection of randomly mutated molecules is subjected to selection or screening procedures. In the latter, discrete regions of a polypeptide, corresponding either to defined structural or functional determinants are subjected to saturating or semi-random mutagenesis and these mutagenized cassettes are re-introduced into the context of the otherwise wild type allele. In one embodiment, PCR mutagenesis can be used. For example, Megaprimer PCR can be used (O. H. Landt, 1990. Gene 96:125-128).

In preferred embodiments, a MarA family polypeptide excludes one or more of XylS, AraC, and MeIR. In other preferred embodiments, a MarA family polypeptide is involved in antibiotic resistance. In particularly preferred embodiments, a MarA family polypeptide is selected from the group consisting of: MarA, RamA, AarP, Rob, SoxS, and PqrA.

The language “activity of a transcription factor” includes the ability of a transcription factor to interact with DNA, e.g., to bind to a transcription factor responsive promoter, or to initiate transcription from such a promoter. The language expressly includes the activities of AraC family polypeptides, HTH proteins and MarA family polypeptides.

The language “activity of a MarA family polypeptide” includes the ability of the MarA family polypeptide to interact with DNA, e.g., to bind to a MarA family polypeptide responsive promoter, or to initiate transcription from such a promoter. MarA functions both as a transcriptional activator (e.g., upregulating genes such as inaA, galT, micF, etc.) and as a repressor (e.g., downregulating genes such as fecA, purA, guaB, etc.) (Alekshun, 1997, Antimicrob. Agents Chemother. 41:2067-2075; Barbosa & Levy, J. Bact. 2000, Vol. 182, p. 3467-3474; Pomposiello et al. J. Bact. 2001, Vol 183, p. 3890-3902).

The language “transcription factor responsive element” includes a nucleic acid sequence which can interact with a transcription factor (e.g., promoters or enhancers or operators) which are involved in initiating transcription of an operon in a microbe. Transcription factor responsive elements responsive to various transcription factors are known in the art and additional responsive elements can be identified by one of ordinary skill in the art. For example, microarray analysis can be used to identify genes that are regulated by a transcription factor of interest. For interest, genes regulated by a transcription factor would be expressed at higher levels in wild type cells than in cells which are deleted for the transcription factor. In addition, genes responsive to a given transcription factor would comprise one or more target sequences responsive to the transcription factor in their promoter regions (Lyons et al. 2000. PNAS 97:7957). Exemplary responsive elements include: araBAD, araE, araFGH (responsive to AraC); melBAD (responsive to MelR); rhaSR (responsive to RhaR); rahBAD, rhaT (responsive to RhaS); Pm (responsive to XyIS); fumC, inaA, micF, nfo, pai5, sodA, toIC, acrAB, fldA, fpr, mar, poxB, ribA, and zwf (responsive to MarA, SoxS, Rob); and coo, rns (responsive to Rns).

The language “marA family polypeptide responsive element” includes a nucleic acid sequence which can interact with marA, e.g., promoters or enhancers which are involved in regulating transcription of a nucleic acid sequence in a microbe. MarA responsive elements comprise approximately 16 base pair marbox sequence, the sequence critical for the binding of MarA to its target. In addition, a secondary site, the accessory marbox, upstream of the primary marbox contributes to basal and derepressed mar transcription. A marbox may be situated in either the forward or backward orientation. (Martin, 1999, Mol. Microbiol. 34:431-441). In the marRAB operon, the marbox is in the backward orientation and is thus located on the sense strand with respect to marRAB (Martin, 1999, Mol. Microbiol. 34:431-441). Subtle differences within the marbox sequence of particular promoters may account for differential regulation by MarA and other related, e.g., SoxS and Rob, transcription factors (Martin, 2000, Mol Microbiol; 35(3):623-34). In one embodiment, MarA family responsive elements are promoters that are structurally or functionally related to a marA promoter, e.g., interact with MarA or a protein related to MarA. Preferably, the marA family polypeptide responsive element is a marRAB promoter. For example, in the mar operon, several promoters are marA family polypeptide responsive promoters as defined herein, e.g., the 405-bp ThaI fragment from the marO region is a marA family responsive promoter (Cohen et al. 1993. J Bact. 175:7856). In addition, MarA has been shown to bind to a 16 bp MarA binding site (referred to as the “marbox” within marO (Martin et al. 1996. J. Bacteriol. 178:2216). MarA also affects transcription from the acrAB; micF; mir 1,2,3; s/p; nfo; inaA; fpr; sodA; soi-17,19; zwf fumc; or rpsF promoters (Alekshun and Levy. 1997. Antimicrobial Agents and Chemother. 41:2067). Other marA family responsive promoters are known in the art and include: araBAD, araE, araFGH and araC, which are activated by AraC; Pm, which is activated by XylS; melAB which is activated by MelR; and oriC which is bound by Rob.

The language “MarA family polypeptide responsive promoter” also includes portions of the above promoters which are sufficient to activate transcription upon interaction with a MarA family member protein. The portions of any of the MarA family polypeptide-responsive promoters which are minimally required for their activity can be easily determined by one of ordinary skill in the art, e.g., using mutagenesis. Exemplary techniques are described by Gallegos et al. (1996, J. Bacteriol. 178:6427). A “MarA family polypeptide responsive promoter” also includes non-naturally occurring variants of MarA family polypeptide responsive promoters which have the same function as naturally occurring MarA family promoters. Preferably such variants have at least 30% or greater, 40% or greater, or 50% or greater, nucleotide sequence identity with a naturally occurring MarA family polypeptide responsive promoter. In preferred embodiments, such variants have at least about 70% nucleotide sequence identity with a naturally occurring MarA family polypeptide responsive promoter. In more preferred embodiments, such variants have at least about 80% nucleotide sequence identity with a naturally occurring MarA family polypeptide responsive promoter. In particularly preferred embodiments, such variants have at least about 90% nucleotide sequence identity and preferably at least about 95% nucleotide sequence identity with a naturally occurring MarA family polypeptide responsive promoter. In yet other embodiments nucleic acid molecules encoding variants of MarA family polypeptide responsive promoters are capable of hybridizing under stringent conditions to nucleic acid molecules encoding naturally occurring MarA family polypeptide responsive promoters.

In one embodiment, the methods described herein can employ molecules identified as responding to the transcription factors of the invention, i.e., molecules in a regulon whose expression is controlled by the transcription factor. For example, compounds that modulate transcription of genes that are directly modulated by a microbial transcription factor (e.g., a marA family transcription factor) can be used to modulate virulence of a microbe or modulate infection by a microbe. In another embodiment, such genes can be identified as important in controlling virulence using the methods described herein. As used herein, the term “regulon” includes two or more loci in two or more different operons whose expression is regulated by a common repressor or activator protein.

The term “interact” includes close contact between molecules that results in a measurable effect, e.g., the binding of one molecule with another. For example, a MarA family polypeptide can interact with a MarA family polypeptide responsive element and alter the level of transcription of DNA. Likewise, compounds can interact with a MarA family polypeptide and alter the activity of a MarA family polypeptide.

The term “inducible promoter” includes promoters that are activated to induce the synthesis of the genes they control. As used herein, the term “constitutive promoter” includes promoters that do not require the presence of an inducer, e.g., are continuously active.

The terms “heterologous DNA” or “heterologous nucleic acid” includes DNA that does not occur naturally in the cell (e.g., as part of the genome) in which it is present or which is found in a location or locations in the genome that differ from that in which it occurs in nature or which is operatively linked to DNA to which it is not normally linked in nature (i.e., a gene that has been operatively linked to a heterologous promoter). Heterologous DNA is 1) not naturally occurring in a particular position (e.g., at a particular position in the genome) or 2) is not endogenous to the cell into which it is introduced, but has been obtained from another cell. Heterologous DNA can be from the same species or from a different species. Any DNA that one of skill in the art would recognize or consider as heterologous or foreign to the cell in which is expressed is herein encompassed by the term heterologous DNA.

The terms “heterologous protein,” “recombinant protein,” and “exogenous protein” are used interchangeably throughout the specification and refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid molecule.

The term “microbe” includes microorganisms expressing or made to express a transcription factor, araC family polypeptide, HTH protein, or a marA family polypeptide. “Microbes” are of some economic importance, e.g., are environmentally important or are important as human pathogens. For example, in one embodiment microbes cause environmental problems, e.g., fouling or spoilage, or perform useful functions such as breakdown of plant matter. In another embodiment, microbes are organisms that live in or on mammals and are medically important. Preferably microbes are unicellular and include bacteria, fungi, or protozoa. In another embodiment, microbes suitable for use in the invention are multicellular, e.g., parasites or fungi. In preferred embodiments, microbes are pathogenic for humans, animals, or plants. Microbes may be used as intact cells or as sources of materials for cell-free assays. In one embodiment, the microbes include prokaryotic organisms. In other embodiments, the microbes include eukaryotic organisms. Exemplary bacteria that comprise MarA homologs include the following:

MarA

E. coli
UPEC (uropathogenic)
EPEC (enteropathogenic)
Salmonella enterica
Cholerasuis (septicemia)
Enteritidis enteritis
Typhimurium enteritis
Typhimurium (multi-drug resistant)
Yersinia enterocolitica
Yersinia pestis
Pseudomonas aeruginosa
Enterobacter spp.
Klebsiella sp.
Proteus spp.
Vibrio cholerae
Shigella sp.
Providencia stuartii
Neisseria meningitidis
Mycobacterium tuberculosis
Mycobacterium leprae
Staphylococcus aureus
Streptococcus pyogenes
Enterococcus faecalis
Bordetella pertussis
Bordetella bronchiseptica

The term “selective marker” includes polypeptides that serve as indicators, e.g., provide a selectable or screenable trait when expressed by a cell. The term “selective marker” includes both selectable markers and counterselectable markers. As used herein, the term “selectable marker” includes markers that result in a growth advantage when a compound or molecule that fulfills the test parameter of the assay is present. The term “counterselectable marker” includes markers that result in a growth disadvantage unless a compound or molecule is present which disrupts a condition giving rise to expression of the counterselectable marker. Exemplary selective markers include cytotoxic gene products, gene products that confer antibiotic resistance, gene products that are essential for growth, gene products that confer a selective growth disadvantage when expressed in the presence of a particular metabolic substrate (e.g., the expression of the URA3 gene confers a growth disadvantage in the presence of 5-fluoroorotic acid).

As used herein the term “reporter gene” includes any gene which encodes an easily detectable product which is operably linked to a regulatory sequence, e.g., to a transcription factor responsive promoter. By operably linked it is meant that under appropriate conditions an RNA polymerase may bind to the promoter of the regulatory region and proceed to transcribe the nucleotide sequence such that the reporter gene is transcribed. In preferred embodiments, a reporter gene consists of the transcription factor responsive promoter linked in frame to the reporter gene. In certain embodiments, however, it may be desirable to include other sequences, e.g., transcriptional regulatory sequences, in the reporter gene construct. For example, modulation of the activity of the promoter may be effected by altering the RNA polymerase binding to the promoter region, or, alternatively, by interfering with initiation of transcription or elongation of the mRNA. Thus, sequences which are herein collectively referred to as transcriptional regulatory elements or sequences may also be included in the reporter gene construct. In addition, the construct may include sequences of nucleotides that alter translation of the resulting mRNA, thereby altering the amount of reporter gene product.

Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); PhoA, alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368) and green fluorescent protein (U.S. Pat. No. 5,491,084; WO96/23898).

In certain embodiments of the invention it will be desirable to obtain “isolated or recombinant” nucleic acid molecules transcription factors or mutant forms thereof. The term “isolated or recombinant” includes nucleic acid molecules which have been, e.g., (1) amplified in vitro by, for example, polymerase chain reaction (PCR); (2) recombinantly produced by cloning, or (3) purified, as by cleavage and gel separation; or (4) synthesized by, for example, chemical synthesis. Such a nucleic acid molecule is isolated from the sequences which naturally flank it in the genome and from cellular components.

In yet other embodiments of the invention, it will be desirable to obtain a substantially purified or recombinant transcription factor. Such polypeptides, for example, can be purified from cells which have been engineered to express an isolated or recombinant nucleic acid molecule which encodes a transcription factor. For example, as described in more detail below, a bacterial cell can be transformed with a plasmid which encodes a transcription factor. The transcription factor can then be purified from the bacterial cells and used, for example, in the cell-free assays described herein or known in the art.

As used herein, the term “antibiotic” includes antimicrobial agents isolated from natural sources or chemically synthesized. The term “antibiotic” refers to antimicrobial agents for use in human therapy. Preferred antibiotics include: tetracycline, fluoroquinolones, chloramphenicol, penicillins, cephalosporins, puromycin, nalidixic acid, and rifampin.

The term “test compound” includes any reagent or test agent which is employed in the assays of the invention and assayed for its ability to influence the activity of a transcription factor, e.g., an AraC family polypeptide, an HTH protein, or a MarA family polypeptide, e.g., by binding to the polypeptide or to a molecule with which it interacts. More than one compound, e.g., a plurality of compounds, can be tested at the same time for their ability to modulate the activity of a transcription factor, e.g., an AraC family polypeptide, an HTH protein, or a MarA family polypeptide, activity in a screening assay. In an advantageous embodiment, the test compound is a MarA family modulating compound.

Test compounds that can be tested in the subject assays include antibiotic and non-antibiotic compounds. In one embodiment, test compounds include candidate detergent or disinfectant compounds. Exemplary test compounds which can be screened for activity include, but are not limited to, peptides, non-peptidic compounds, nucleic acids, carbohydrates, small organic molecules (e.g., polyketides), and natural product extract libraries. The term “non-peptidic test compound” includes compounds that are comprised, at least in part, of molecular structures different from naturally-occurring L-amino acid residues linked by natural peptide bonds. However, “non-peptidic test compounds” also include compounds composed, in whole or in part, of peptidomimetic structures, such as D-amino acids, non-naturally-occurring L-amino acids, modified peptide backbones and the like, as well as compounds that are composed, in whole or in part, of molecular structures unrelated to naturally-occurring L-amino acid residues linked by natural peptide bonds. “Non-peptidic test compounds” also are intended to include natural products.

In one embodiment, small molecules can be used as test compounds. The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. 1998. Science 282:63), and natural product extract libraries. In another embodiment, the compounds are small, organic non-peptidic compounds. In a further embodiment, a small molecule is not biosynthetic.

The term “antagonist” includes transcription factor modulating compounds (e.g., AraC family polypeptide modulating compounds, HTH protein modulating compounds, MarA family polypeptide modulating compounds, etc.) which inhibit the activity of a transcription factor by binding to and inactivating the transcription factor (e.g., an AraC family modulating compound, an MarA family polypeptide modulating compound, etc.), by binding to a nucleic acid target with which the transcription factor interacts (e.g., for MarA, a marbox), by disrupting a signal transduction pathway responsible for activation of a particular regulon (e.g., for Mar, the inactivation of MarR or activation of MarA synthesis), and/or by disrupting a critical protein-protein interaction (e.g., MarA-RNA polymerase interactions that are required for MarA to function as a transcription factor.) Antagonists may include, for example, naturally or chemically synthesized compounds such as small cell permeable organic molecules, nucleic acid interchelators, peptides, etc.

The term “agonist” includes transcription factor modulating compounds (e.g., AraC family polypeptide modulating compounds, HTH protein modulating compounds, MarA family polypeptide modulating compounds, etc.) which promote the activity of a transcription factor by binding to and activating the transcription factor (e.g., an AraC family modulating compound, an MarA family polypeptide modulating compound, etc.), by binding to a nucleic acid target with which the transcription factor interacts (e.g., for MarA, a marbox), by facilitating a signal transduction pathway responsible for activation of a particular regulon (e.g., for Mar, the inactivation of MarR or activation of MarA synthesis), and/or by facilitating a critical protein-protein interaction (e.g., MarA-RNA polymerase interactions that are required for MarA to function as a transcription factor.) Agonists may include, for example, naturally or chemically synthesized compounds such as small cell permeable organic molecules, nucleic acid interchelators, peptides, etc.

II. MarA Family polypeptide Helix-Turn-Helix Domains

Helix-turn-helix domains are known in the art and have been implicated in DNA binding (Ann Rev. of Biochem. 1984. 53:293). An example of the consensus sequence for a helix-turn domain can be found in Brunelle and Schleif (1989, J. Mol. Biol. 209:607). The domain has been illustrated by the sequence XXXPhoAlaXXPhoGlyPhoXXXXPhoXXPhoXX, where X is any amino acid and Pho is a hydrophobic amino acid.

The crystal structure of MarA has been determined and the first (most amino terminal) HTH domain of MarA has been identified as comprising from about amino acid 31 to about amino acid 52 and the second HTH domain of MarA has been identified as comprising from about amino acid 79 to about amino acid 102 (Rhee et al. 1998. Proc. Natl. Acad. Sci. USA. 95:10413).

Locations of the helix-turn-helix domains in other MarA family members as well as other HTH proteins can easily be found by one of skill in the art. For example using the MarA protein sequence and an alignment program, e.g., the ProDom program or other programs known in the art, a portion of the MarA amino acid sequence e.g., comprising one or both HTH domains of MarA (such as from about amino acid 30 to about amino acid 107 of MarA) to produce an alignment. Using such an alignment, the amino acid sequences corresponding to the HTH domains of MarA can be identified in other MarA family member proteins. An exemplary consensus sequence for the first helix-turn-helix domain of a MarA family polypeptide can be illustrated as XXXXAXXXXXSXXXLXXXFX, where X is any amino acid. An exemplary consensus sequence for the second helix-turn-helix domain of a MarA family polypeptide is illustrated as XXIXXIAXXXGFXSXXXFXXX[F/Y], where X is any amino acid. Preferably, a MarA family polypeptide first helix-turn-helix domain comprises the consensus sequence E/D-X-V/L-A-D/E-X-A/S-G-X-S-X3-L-Q-X2-F-K/R/E-X2-T/I. Preferably, a MarA family polypeptide second helix-turn-helix domain comprises the consensus sequence 1-X-D-1-A-X3-G-F-X-S-X2-F-X3-F-X4.

In an embodiment, a MarA family member HTH domain is a MarA HTH domain. The first and second helix-turn-helix domains of MarA are, respectively, EKVSERSGYSKWHLQRMFKKET and ILYLAERYGFESQQTLTRTFKNYF. Other exemplary MarA family helix-turn-helix domains include: about amino acid 210 to about amino acid 229 and about amino acid 259 to about amino acid 278 of MelR; about amino acid 196 to about amino acid 215 and about amino acid 245 to about amino acid 264 of AraC; and about amino acid 230 to about amino acid 249 (or 233-253) and about amino acid 281 to about amino acid 301 (or 282-302) of XylS (see e.g., Brunelle et al. 1989. J. Mol. Biol. 209:607; Niland et al. 1996. J. Mol. Biol. 264:667; Gallegos et al. 1997. Microbiology and Molecular Biology Reviews. 61:393).

“MarA family polypeptide helix-turn-helix domains” are derived from or are homologous to the helix-turn-helix domains found in the MarA family polypeptides as described supra. In preferred embodiments, a MarA family polypeptide excludes one or more of XylS, AraC, and MelR. In particularly preferred embodiments, a MarA family polypeptide is selected from the group consisting of: MarA, RamA, AarP, Rob, SoxS, and PqrA.

Both of the helix-turn-helix domains present in MarA family polypeptides are in the carboxy terminal end of the protein. Proteins or portions thereof comprising either or both of these domains can be used in the instant methods. In certain embodiments, a polypeptide which is used in screening for compounds comprises the helix-turn-helix domain most proximal to the carboxy terminus (HTH2) of the MarA family polypeptide from which it is derived. In other embodiments, such a polypeptide comprises the helix-turn-helix domain most proximal to the amino terminus (HTH1) of the MarA family polypeptide from which it is derived. In one embodiment, other polypeptide sequences may also be present, e.g., sequences that might facilitate immobilizing the domain on a support, or, alternatively, might facilitate the purification of the domain.

In an embodiment, such a polypeptide consists essentially of the helix-turn-helix domain most proximal to the carboxy terminus of the MarA family polypeptide from which it is derived. In other preferred embodiments, such a polypeptide consists essentially of the helix-turn-helix domain most proximal to the amino terminus of the MarA family polypeptide from which it is derived.

In an embodiment, such a polypeptide consists of the helix-turn-helix domain most proximal to the carboxy terminus of the AraC family polypeptide or MarA family polypeptide from which it is derived. In other preferred embodiments, such a polypeptide consists of the helix-turn-helix domain most proximal to the amino terminus of the AraC family polypeptide or MarA family polypeptide from which it is derived.

MarA family polypeptide or AraC family polypeptide helix-turn-helix domains can be made using techniques which are known in the art. The nucleic acid and amino acid sequences of transcription factors, such as MarA family polypeptides, are available, for example, from GenBank. Using this information, the helix-turn-helix consensus motif and mutational analysis provided herein, one of ordinary skill in the art can identify MarA family or AraC family polypeptide helix-turn-helix domains.

In certain embodiments of the invention it will be desirable to obtain “isolated or recombinant” nucleic acid molecules encoding transcription factors or portions thereof (e.g., HTH protein helix-turn-helix domains, AraC family helix-turn-helix domains, MarA family helix-turn-helix domains or mutant forms thereof). By “isolated or recombinant” is meant a nucleic acid molecule which has been (1) amplified in vitro by, for example, polymerase chain reaction (PCR); (2) recombinantly produced by cloning, or (3) purified, as by cleavage and gel separation; or (4) synthesized by, for example, chemical synthesis. Such a nucleic acid molecule is isolated from the sequences which naturally flank it in the genome and from cellular components.

The isolated or recombinant nucleic acid molecules encoding transcription factors (e.g., HTH protein helix-turn-helix domains, AraC family helix-turn-helix domains, MarA family helix-turn-helix domains or mutant forms thereof) can then, for example, be utilized in binding assays, can be expressed in a cell, or can be expressed on the surface of phage, as discussed further below.

In yet other embodiments of the invention, it will be desirable to obtain a substantially purified or recombinant HTH protein helix-turn-helix domains (e.g., MarA family helix-turn-helix domains or mutant forms thereof). Such polypeptides, for example, can be purified from cells which have been engineered to express an isolated or recombinant nucleic acid molecule which encodes a HTH protein helix-turn-helix domain (e.g., MarA family helix-turn-helix domain or mutant forms thereof). For example, as described in more detail below, a bacterial cell can be transformed with a plasmid which encodes a MarA family helix-turn-helix domain. The MarA family helix-turn-helix protein can then be purified from the bacterial cells and used, for example, in the cell-free assays described herein.

Purification of a HTH protein helix-turn-helix domain (e.g., MarA family helix-turn-helix domain) can be accomplished using techniques known in the art. For example, column chromatography could be used, or antibodies specific for the domain or for a polypeptide fused to the domain can be employed, for example on a column or in a panning assay.

In preferred embodiments, cells used to express HTH protein helix-turn-helix domains (e.g., MarA family helix-turn-helix domains or mutant forms thereof) for purification, e.g., host cells, comprise a mutation which renders any endogenous HTH proteins nonfunctional or causes the endogenous protein to not be expressed. In other embodiments, mutations may also be made in MarR or related genes of the host cell, such that repressor proteins which bind to the same promoter as a MarA family polypeptide are not expressed by the host cell.

In certain embodiments of the invention, it will be desirable to use a mutant form of a HTH protein helix-turn helix domain, e.g., a non-naturally occurring form of a MarA family helix-turn-helix domain which has altered activity, e.g., does not retain wild type MarA family polypeptide helix-turn-helix domain activity, or which has reduced activity or which is more active when compared to a wild-type MarA family polypeptide helix-turn-helix domain.

Such mutant forms can be made using techniques which are well known in the art. For example, random mutagenesis can be used. Using random mutagenesis one can mutagenize an entire molecule or one can proceed by cassette mutagenesis. In the former instance, the entire coding region of a molecule is mutagenized by one of several methods (chemical, PCR, doped oligonucleotide synthesis) and that collection of randomly mutated molecules is subjected to selection or screening procedures. In the second approach, discrete regions of a protein, corresponding either to defined structural or functional determinants (e.g., the first or second alpha helix of a helix-turn-helix domain) are subjected to saturating or semi-random mutagenesis and these mutagenized cassettes are re-introduced into the context of the otherwise wild type allele.

In a preferred embodiment, PCR mutagenesis is used. For example, Example 2 of U.S. Ser. No. 11/115,024, describes the use of Megaprimer PCR (O. H. Landt, Gene 96:125-128) used to introduce an NheI restriction site into the centers of both the helix A (position 1989) and helix B (position 2016) regions of the marA gene.

In one embodiment, such mutant helix-turn-helix domains comprise one or more mutations in the helix-turn-helix domain most proximal to the carboxy terminus (HTH2) of the MarA family polypeptide molecule. In a preferred embodiment, the mutation comprises an insertion into helix A and helix B of the helix-turn-helix domain most proximal to the carboxy terminus of the MarA family polypeptide. In one embodiment, such mutant helix-turn-helix domains comprise one or more mutations in the helix-turn-helix domain most proximal to the amino terminus (HTH1) of the MarA family polypeptide molecule. In a preferred embodiment, the mutation comprises an insertion into helix A and helix B of the helix-turn-helix domain most proximal to the amino terminus of the MarA family polypeptide. In particularly preferred embodiments, the mutation is selected from the group consisting of: an insertion at an amino acid corresponding to about position 33 of MarA and an insertion at an amino acid position corresponding to about position 42 of MarA. “Corresponding” amino acids can be determined, e.g., using an alignment of the helix-turn-helix domains.

Such mutant forms of MarA family helix-turn-helix motifs are useful as controls to verify the specificity of antiinfective compounds for a MarA family helix-turn-helix domain or as controls for the identification of genetic loci which affect resistance to antiinfectives. For example, the mutant MarA family helix-turn-helix domains described in the appended Examples demonstrate that insertional inactivation of MarA at either helix A or helix B in the first HTH domain abolished the multidrug resistance phenotype in both E. coli and M. smegmatis. By the use of an assay system such as that described in Example 2, which demonstrates the ability of MarA family polypeptide helix-turn-helix domains to increase antibiotic resistance and that mutant forms of these domains do not have the same effect, one can clearly show that the response of any genetic loci identified is specific to a MarA family helix-turn-helix domain.

### III. Expression of Polypeptide or Portions Thereof

Nucleic acids encoding transcription factors, such as AraC family polypeptides, HTH proteins, e.g., MarA family polypeptides or selectable markers (or portions thereof that retain an activity of the full-length polypeptide, e.g., are capable of binding to a transcription factor responsive element or retain their indicator function) can be expressed in cells using vectors. Almost any conventional delivery vector can be used. Such vectors are widely available commercially and it is within the knowledge and discretion of one of ordinary skill in the art to choose a vector which is appropriate for use with a given microbial cell. The sequences encoding these domains can be introduced into a cell on a self-replicating vector or may be introduced into the chromosome of a microbe using homologous recombination or by an insertion element such as a transposon.

These nucleic acids can be introduced into microbial cells using standard techniques, for example, by transformation using calcium chloride or electroporation. Such techniques for the introduction of DNA into microbes are well known in the art. In one embodiment, a nucleic acid molecule which has been amplified in vitro by, for example, polymerase chain reaction (PCR); recombinantly produced by cloning, or) purified, as by cleavage and gel separation; or synthesized by, for example, chemical synthesis can be used to produce MarA family polypeptides (George, A. M. & Levy, S. B. (1983) J. Bacteriol. 155, 541-548; Cohen, S. P. et al. (1993) J. Infect. Dis. 168, 484-488; Cohen, S. P et al. (1993) J Bacteriol. 175, 1484-1492; Sulavick, M. C. et al. (1997) J. Bacteriol. 179, 1857-1866).

Host cells can be genetically engineered to incorporate nucleic acid molecules of the invention. In one embodiment nucleic acid molecules specifying transcription factors can be placed in a vector. The term “vector” refers to a nucleic acid molecule capable of transporting another nucleic acid molecule to which it has been linked. The term “expression vector” or “expression system” includes any vector, (e.g., a plasmid, cosmid or phage chromosome) containing a gene construct in a form suitable for expression by a cell (e.g., linked to a promoter). In the present specification, “plasmid” and “vector” are used interchangeably, as a plasmid is a commonly used form of vector. Moreover, the invention is intended to include other vectors which serve equivalent functions. A great variety of expression systems can be used to produce the polypeptides of the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Appropriate vectors are widely available commercially and it is within the knowledge and discretion of one of ordinary skill in the art to choose a vector which is appropriate for use with a given host cell. The sequences encoding a transcription factor, such as, for example, MarA family polypeptides, can be introduced into a cell on a self-replicating vector or may be introduced into the chromosome of a microbe using homologous recombination or by an insertion element such as a transposon.

The expression system constructs may contain control regions that regulate expression. “Transcriptional regulatory sequence” is a generic term to refer to DNA sequences, such as initiation signals, enhancers, operators, and promoters, which induce or control transcription of polypeptide coding sequences with which they are operably linked. It will also be understood that a recombinant gene encoding a transcription factor gene, e.g., an HTH protein gene or an AraC family polypeptide, e.g., MarA family polypeptide, can be under the control of transcriptional regulatory sequences which are the same or which are different from those sequences which control transcription of the naturally-occurring transcription factor gene. Exemplary regulatory sequences are described in Goeddel; Gene Expression Technology: Methods in Enzymology 185, Academic Press, San Diego, Calif. (1990). For instance, any of a wide variety of expression control sequences, that control the expression of a DNA sequence when operatively linked to it, may be used in these vectors to express DNA sequences encoding the polypeptide.

Generally, any system or vector suitable to maintain, propagate or express nucleic acid molecules and/or to express a polypeptide in a host may be used for expression in this regard. The appropriate DNA sequence may be inserted into the expression system by any of a variety of well-known and routine techniques, such as, for example, those set forth in Sambrook et al., Molecular Cloning, A Laboratory Manual, (supra).

Exemplary expression vectors for expression of a gene encoding a polypeptide and capable of replication in a bacterium, e.g., a gram positive, gram negative, or in a cell of a simple eukaryotic fungus such as a Saccharomyces or, Pichia, or in a cell of a eukaryotic organism such as an insect, a bird, a mammal, or a plant, are known in the art. Such vectors may carry functional replication-specifying sequences (replicons) both for a host for expression, for example a Streptomyces, and for a host, for example, E. coli, for genetic manipulations and vector construction. See, e.g., U.S. Pat. No. 4,745,056. Suitable vectors for a variety of organisms are described in Ausubel, F. et al., Short Protocols in Molecular Biology, Wiley, New York (1995), and for example, for Pichia, can be obtained from Invitrogen (Carlsbad, Calif.).

Useful expression control sequences, include, for example, the early and late promoters of SV40, adenovirus or cytomegalovirus immediate early promoter, the lac system, the trp system, the TAC or TRC system, T7 promoter whose expression is directed by T7, RNA polymerase, the major operator and promoter regions of phage lambda, the control regions for fd coat polypeptide, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. A useful translational enhancer sequence is described in U.S. Pat. No. 4,820,639.

In one embodiment, an inducible promoter will be employed to express a polypeptide of the invention. For example, in one embodiment, trp (induced by tryptophan), tac (induced by lactose), or tet (induced by tetracycline) can be used in bacterial cells, or GAL1 (induced by galactose) can be used in yeast cell.

In another embodiment, a constitutive promoter can be used to express a polypeptide of the invention.

It should be understood that the design of the expression vector may depend on such factors as the choice of the host cell to be transformed and/or the type of polypeptide desired to be expressed. Representative examples of appropriate hosts include bacterial cells, such as gram positive, gram negative cells; fungal cells, such as yeast cells and Aspergillus cells; insect cells such as Drosophila S2 and Spodoplera Sf9 cells; animal cells such as CHO, COS, HeLa, C127, 3T3, BHK, 293 and Bowes melanoma cells; and plant cells.

In one embodiment, cells used to express heterologous polypeptides of the invention, comprise a mutation which renders one or more endogenous transcription factors, such as a AraC family polypeptide or a MarA family polypeptide, nonfunctional or causes one or more endogenous polypeptide to not be expressed. Manipulation of the genetic background in this manner allows for screening for compounds that modulate specific transcription factors, such as MarA family members or AraC family members, or more than one transcription factors.

In other embodiments, mutations may also be made in other related genes of the host cell, such that there will be no interference from the endogenous host loci. In yet another embodiment, a mutation may be made in a chromosomal gene to create a heterotroph.

Introduction of a nucleic acid molecule into the host cell (“transformation”) can be effected by methods described in many standard laboratory manuals, such as Davis et al., Basic Methods In Molecular Biology, (1986) and Sambrook et al., Molecular Cloning: A Laboratory Manual, 2nd Ed., Cold Spring Harbor Laboratory Press, Cold Spring Harbor, N.Y. (1989). Examples include calcium phosphate transfection, DEAE-dextran mediated transfection, transvection, microinjection, cationic lipid-mediated transfection, electroporation, transduction, scrape loading, ballistic introduction and infection.

Purification of polypeptides, e.g., recombinantly expressed polypeptides, can be accomplished using techniques known in the art. For example, if the polypeptide is expressed in a form that is secreted from cells, the medium can be collected. Alternatively, if the polypeptide is expressed in a form that is retained by cells, the host cells can be lysed to release the polypeptide. Such spent medium or cell lysate can be used to concentrate and purify the polypeptide. For example, the medium or lysate can be passed over a column, e.g., a column to which antibodies specific for the polypeptide have been bound. Alternatively, such antibodies can be specific for a second polypeptide which has been fused to the first polypeptide (e.g., as a tag) to facilitate purification of the first polypeptide. Other means of purifying polypeptides are known in the art.

IV. Methods for Identifying Antiinfective Compounds which Modulate an Activity of a Transcription Factor

Transcription factor agonists and antagonists can be assayed in a variety of ways. For example, in one embodiment, the invention provides for methods for identifying a compound which modulates an transcription factor, e.g., by measuring the ability of the compound to interact with an transcription factor nucleic acid molecule or an transcription factor polypeptide or the ability of a compound to modulate the activity or expression of an transcription factor polypeptide. Furthermore, the ability of a compound to modulate the binding of an transcription factor polypeptide or transcription factor nucleic acid molecule to a molecule to which they normally bind, e.g., a nucleic acid or protein molecule can be tested.

In one embodiment, a transcription factor and its cognate DNA sequence can be present in a cell free system, e.g., a cell lysate and the effect of the compound on that interaction can be measured using techniques known in the art.

In a preferred embodiment, the assay system is a cell-based system. Compounds identified using the subject methods are useful, e.g., to interfere with the ability of a microbe to grow in a host and/or in reducing microbial virulence and, thereby, and in reducing the ability of the microbe to cause infection in a host.

The ability of the test compound to modulate the expression and/or activity of a transcription factor can be determined in a variety of ways. Exemplary methods which can be used in the instant assays are known in the art and are described, e.g., in U.S. Pat. No. 5,817,793 and WO 99/61579. Other exemplary methods are described in more detail below.

In one embodiment, the invention provides for methods of identifying a test compound which modulates the expression and/or activity of a transcription factor, (e.g., an HTH protein, a MarA family polypeptide, an AraC family polypeptide, etc.) by contacting a cell expressing a transcription factor (or portion thereof) with a test compound under conditions which allow interaction of the test compound with the cell.

### Assays

In one embodiment, the expression of a selectable marker that confers a selective growth disadvantage or lethality is placed under the direct control of a MarA responsive element in a cell expressing marA.

In one embodiment, marA is plasmid encoded. In one embodiment, the genetic background of the host organism is manipulated, e.g., to delete one or more chromosomal marA genes or marA homolog genes.

In one embodiment, expression of marA is controlled by a highly regulated and inducible promoter. For example, in one embodiment, a promoter selected from the group consisting of trp, tac, or tet in bacterial cells or GAL1 in yeast cells can be used.

In another embodiment, expression of marA is constitutive.

In one embodiment, a selective marker is a cytotoxic gene product (e.g., ccdB).

In another embodiment, a selective marker is a gene that confers antibiotic resistance (e.g., kan, cat, or bla).

In another embodiment, a selective marker is an essential gene (e.g., purA or guaB in a purine or guanine heterotroph).

In still another embodiment, a selective marker is a gene that confers a selective growth disadvantage in the presence of a particular metabolic substrate (e.g., the expression of URA3 in the presence of 5-fluoroorotic acid [5-FOA] in yeast).

In one embodiment, compounds that modulate transcription factors (e.g., HTH proteins, AraC family polypeptides, or MarA family polypeptides) are identified using a one-hybrid screening assay. As used herein, the term “one-hybrid screen” as used herein includes assays that detect the disruption of protein-nucleic acid interactions. These assays will identify agents that interfere with the binding of a transcription factor (e.g., an HTH protein, a AraC family polypeptide, or a MarA family polypeptide) to a particular target, e.g., DNA containing, for MarA, a marbox, at the level of the target itself, e.g., by binding to the target and preventing the transcriptional activation factor from interacting with or binding to this site.

In another embodiment, compounds of the invention are identified using a two-hybrid screening assay. As used herein the term “two-hybrid screen” as used herein includes assays that detect the disruption of protein-protein interactions. Such two hybrid assays can be used to interfere with crucial protein-transcription factor interactions (e.g., HTH protein interactions, AraC family polypeptide interactions, MarA family polypeptide interactions). One example would be to prevent RNA polymerase-MarA family polypeptide contacts, that are necessary for the MarA family polypeptide to function as a transcription factor (either positive acting or negative acting).

In yet another embodiment, compounds of the invention are identified using a three-hybrid screening assay. As used herein the term “three-hybrid screen” as used herein includes assays that will detect the disruption of a signal transduction pathway(s) required for the activation of a particular regulon of interest. In one embodiment, the three-hybrid screen is used to detect disruption of a signal transduction pathway(s) required for the activation of the Mar regulon, i.e., synthesis of MarA. (Li and Park. J. Bact. 181:4824). The assay can be used to identify compounds that may be responsible for activating transcription factor expression, e.g., Mar induction by antibiotics may proceed in this manner.

In one embodiment of the assay, the expression of a selective marker (e.g., ccdB, cat, bla, kan, guaB, URA3) is put under the direct control of an activatable MarA responsive activatable promoter (e.g., inaA, galT, micF). In the absence of Mar A, the expression of the selective marker would be silent. For example, in the case of regulation of the cytotoxic gene ccdB, the gene would be silent and the cells would survive. Synthesis of MarA from an inducible plasmid in a suitable host would result in the activation of the MarA responsive activatable promoter and expression of the selective marker. In the case of ccdB, the gene would be expressed and result in cell death. Compounds that inhibit MarA would be identified as those that permit cell survival under conditions of MarA expression.

In another embodiment, e.g., where the expression of the MarA responsive activatable promoter regulates a gene such as URA3, a different result could be obtained. In this case, in the absence of MarA and thus, in the absence of URA3 expression, cells would grow in the presence of a 5-FOA. Upon activation of MarA expression and thus synthesis of URA3, cells would die following the conversion of 5-FOA to a toxic metabolite by URA3.

In another embodiment, a selectable marker is put under the direct control of a repressible MarA responsive promoter (e.g., fecA). In this example, under conditions of constitutive MarA synthesis, e.g., in a constitutive mar (marc) mutant the expression of the selectable marker would be silent. In the case of ccdB, this would mean that cells would remain viable. Following inactivation of MarA, the selectable marker would be turned on, resulting in cell death.

In another embodiment, a purine or guanine heterotroph can be constructed by the inactivation of the chromosomal guaB or purA genes in E. coli. The guaB or purA gene would then be cloned into a suitable vector, under the control of its natural promoter. This construct would then be transformed into the heterotrophic host. The heterotroph will not grow if MarA expression is constitutive and if cells are grown on media lacking purines or guanine. This can be attributed to MarA mediated repression of guaB or purA synthesis. Candidate inhibiting compounds of MarA can be identified as compounds that restored growth, i.e., relieved MarA mediated repression of guaB and purA expression. In another embodiment, genes that are required for growth in vivo, for example in an animal model of infection.

In preferred embodiments, controls may be included to ensure that any compounds which are identified using the subject assays do not merely appear to modulate the activity of a transcription factor, because they inhibit protein synthesis. For example, if a compound appears to inhibit the synthesis of a protein being translated from RNA which is transcribed upon activation of a MarA family responsive element, it may be desirable to show that the synthesis of a control, e.g., a protein which is being translated from RNA which is not transcribed upon activation of a MarA family responsive element, is not affected by the addition of the same compound. For example, the amount of the MarA family polypeptide being made and compared to the amount of an endogenous protein being made. In another embodiment the microbe could be transformed with another plasmid comprising a promoter which is not a MarA family responsive promoter and a protein operably linked to that promoter. The expression of the control protein could be used to normalize the amount of protein produced in the presence and absence of compound.

### V. Microbes Suitable For Testing

Numerous different microbes are suitable for testing in the instant assays. As such, they may be used as intact cells or as sources of material, e.g., nucleic acid molecules or polypeptides as described herein.

In preferred embodiments, microbes for use in the claimed methods are bacteria, either Gram negative or Gram positive bacteria. More specifically, any bacteria that are shown to become resistant to antibiotics, e.g., to display a Mar phenotype are preferred for use in the claimed methods, or that are infectious or potentially infectious.

Examples of microbes suitable for testing include, but are not limited to, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Yibrio parahaemolyticus, Legionella pneumophila, Listeria monocytogenes, Neisseria gonorrhoeae, Neisseria meningitidis, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus.

In one embodiment, microbes suitable for testing are bacteria from the family Enterobacteriaceae. In preferred embodiments, the compound is effective against a bacteria of a genus selected from the group consisting of: Escherichia, Proteus, Salmonella, Klebsiella, Providencia, Enterobacter, Burkholderia, Pseudomonas, Aeromonas, Haemophilus, Yersinia, Neisseria, and Mycobacteria.

In yet other embodiments, the microbes to be tested are Gram positive bacteria and are from a genus selected from the group consisting of: Lactobacillus, Azorhizobium, Streptomyces, Pediococcus, Photobacterium, Bacillus, Enterococcus, Staphylococcus, Clostridium, and Streptococcus.

In other embodiments, the microbes to be tested are fungi. In a preferred embodiment the fungus is from the genus Mucor or Candida, e.g., Mucor racmeosus or Candida albicans.

In yet other embodiments, the microbes to be tested are protozoa. In a preferred embodiment the microbe is a malaria or cryptosporidium parasite.

### VI. Transcription Factor Modulating Compounds and Test Compounds

Compounds for testing in the instant methods can be derived from a variety of different sources and can be known or can be novel. In one embodiment, libraries of compounds are tested in the instant methods to identify transcriptional activation factor modulating compounds, e.g., HTH protein modulating compounds, AraC family polypeptide modulating compounds, MarA family polypeptide modulating compounds, etc. In another embodiment, known compounds are tested in the instant methods to identify transcription factor modulating compounds (such as, for example, HTH protein modulating compounds, AraC family polypeptide modulating compounds, MarA family polypeptide modulating compounds, etc.). In an embodiment, compounds among the list of compounds generally regarded as safe (GRAS) by the Environmental Protection Agency are tested in the instant methods. In another embodiment, the transcription factors which are modulated by the modulating compounds are of prokaryotic microbes.

A recent trend in medicinal chemistry includes the production of mixtures of compounds, referred to as libraries. While the use of libraries of peptides is well established in the art, new techniques have been developed which have allowed the production of mixtures of other compounds, such as benzodiazepines (Bunin et al. 1992. J. Am. Chem. Soc. 114:10987; DeWitt et al. 1993. Proc. Natl. Acad. Sci. USA 90:6909) peptoids (Zuckermann. 1994. J. Med. Chem. 37:2678) oligocarbamates (Cho et al. 1993. Science. 261:1303), and hydantoins (DeWitt et al. supra). Rebek et al. have described an approach for the synthesis of molecular libraries of small organic molecules with a diversity of 104-105 (Carell et al. 1994. Angew. Chem. Int. Ed. Engl. 33:2059; Carell et al. Angew. Chem. Int. Ed. Engl. 1994. 33:2061).

The compounds of the present invention can be obtained using any of the numerous approaches in combinatorial library methods known in the art, including: biological libraries; spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, non-peptide oligomer or small molecule libraries of compounds (Lam, K. S. Anticancer Drug Des. 1997. 12:145).

Exemplary compounds which can be screened for activity include, but are not limited to, peptides, nucleic acids, carbohydrates, small organic molecules, and natural product extract libraries. In one embodiment, the test compound is a peptide or peptidomimetic. In another, preferred embodiment, the compounds are small, organic non-peptidic compounds.

Other exemplary methods for the synthesis of molecular libraries can be found in the art, for example in: Erb et al. 1994. Proc. Natl. Acad. Sci. USA 91:11422; Horwell et al. 1996 Immunopharmacology 33:68; and in Gallop et al. 1994. J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.). Other types of peptide libraries may also be expressed, see, for example, U.S. Pat. Nos. 5,270,181 and 5,292,646). In still another embodiment, combinatorial polypeptides can be produced from a cDNA library.

In other embodiments, the compounds can be nucleic acid molecules. In preferred embodiments, nucleic acid molecules for testing are small oligonucleotides. Such oligonucleotides can be randomly generated libraries of oligonucleotides or can be specifically designed to reduce the activity of a transcription factor, e.g., a HTH protein, a MarA family polypeptide, or an AraC family polypeptide. For example, in one embodiment, these oligonucleotides are sense or antisense oligonucleotides. In an embodiments, oligonucleotides for testing are sense to the binding site of a particular transcription factor, e.g., a MarA family polypeptide helix-turn-helix domain. Methods of designing such oligonucleotides given the sequences of a particular transcription factor polypeptide, such as a MarA family polypeptide, is within the skill of the art.

In yet another embodiment, computer programs can be used to identify individual compounds or classes of compounds with an increased likelihood of modulating a transcription factor activity, e.g., an HTH protein, a AraC family polypeptide, or a MarA family polypeptide activity. Such programs can screen for compounds with the proper molecular and chemical complementarities with a chosen transcription factor. In this manner, the efficiency of screening for transcription factor modulating compounds in the assays described above can be enhanced.

The invention pertains, per se, to not only the methods for identifying the transcription factor modulating compounds, but to the compounds identified by the methods of the invention as well as methods for using the identified compounds.

### VII. MarA Family Modulating Compounds, and Methods of Use Thereof.

In one embodiment, the invention pertains, at least in part, to a method for reducing antibiotic resistance of a microbial cell, comprising contacting said cell with a transcription factor modulating compound of the formula (I):

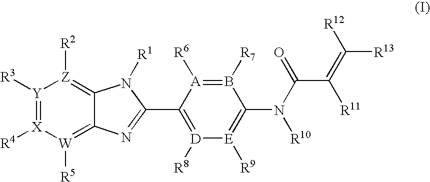

wherein

R¹ is hydroxyl, OCOCO₂H; a straight or branched C₁-C₅ alkyloxy group; or a straight or branched C₁-C₅ alkyl group;

A, B, D, E, W, X, Y and Z are each independently carbon or nitrogen;

wherein R², R³, R⁴, R⁵, R⁶, R⁷, R⁸, R⁹ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen when A, B, D, E, W, X, Y and Z are carbon; or R², R³, R⁴, R⁵, R⁶, R⁷, R⁸, R⁹ are each independently absent or hydroxyl when A, B, D, E, W, X, Y and Z are nitrogen; and

R¹⁰, R¹¹, R¹² and R¹³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen; and pharmaceutically acceptable salts, esters and prodrugs thereof;

provided that when A, B, C, D, E, W, X, Y and Z are each carbon, one of R⁶, R⁷, R⁸, R⁹ is not hydrogen, such that the antibiotic resistance of said microbial cell is reduced.

In another embodiment, the invention pertains, at least in part, to a method for modulating transcription, comprising contacting a transcription factor with a transcription factor modulating compound of the formula (I):

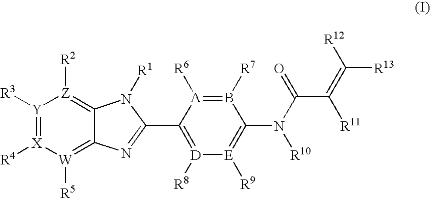

wherein

R¹ is hydroxyl, OCOCO₂H; a straight or branched C₁-C₅ alkyloxy group; or a straight or branched C₁-C₅ alkyl group;

A, B, D, E, W, X, Y and Z are each independently carbon or nitrogen;

wherein R², R³, R⁴, R⁵, R⁶, R⁷, R⁸, R⁹ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen when A, B, D, E, W, X, Y and Z are carbon; or wherein R², R³, R⁴, R⁵, R⁶, R⁷, R⁸, R⁹ are each independently absent or hydroxyl when A, B, D, E, W, X, Y and Z are nitrogen; and

R¹⁰, R¹¹, R¹² and R¹³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen; and pharmaceutically acceptable salts, esters and prodrugs thereof;

provided that when A, B, D, E, W, X, Y and Z are each carbon, one of R⁶, R⁷, R⁸, R⁹ is not hydrogen, such that the transcription is modulated.

In one embodiment, A, B, D, E, W, X, Y and Z are each carbon, R¹ is hydroxy, R², R⁴, R⁵, R¹⁰, R¹¹ and R¹² are each hydrogen, R³ is nitro, R¹³ is aryl, such as halogen substituted phenyl (e.g., 4-fluorophenyl), R⁶ is halogen (e.g., fluorine) and R⁷, R⁸ and R⁹ are hydrogen.

In another embodiment, A, B, D, E, W, X, Y and Z are each carbon, R¹ is hydroxy, R², R⁴, R⁵, R¹⁰, R¹¹ and R¹² are each hydrogen, R³ is nitro, R¹³ is aryl, such as halogen substituted phenyl (e.g., 4-fluorophenyl), R⁶, R⁸ and R⁹ are hydrogen, and R⁹ is halogen (e.g., fluorine).

In a further embodiment, A, B, D, E, W, X, Y and Z are each carbon, R¹ is hydroxy, R², R⁴, R⁵, R¹⁰, R¹¹ and R¹² are each hydrogen, R³ is nitro, R¹³ is aryl, such as halogen substituted phenyl (e.g., 4-fluorophenyl), R⁶, R⁸ and R⁹ are hydrogen, and R⁷ is substituted alkyl (e.g., morpholinylmethyl) or unsubstituted alkyl (e.g., methyl).

In yet another embodiment, A, B, D, E, W, X, Y and Z are each carbon, R¹ is hydroxy, R², R⁴, R⁵, R¹⁰, R¹¹ and R¹² are each hydrogen, R³ is nitro, R¹³ is aryl, such as halogen substituted phenyl (e.g., 4-fluorophenyl), R⁶, R⁷ and R⁹ are each hydrogen and R⁸ is alkoxy (e.g., methoxy).

In one embodiment, A, B, D, E, W, X, Y and Z are each carbon, R¹ is hydroxy, R², R⁴, R⁵, R¹⁰, R¹¹ and R¹² are each hydrogen, R³ is nitro and R¹³ is aryl, such as alkyl substituted phenyl (e.g., 4-methylphenyl). In one embodiment, R⁶, R⁸ and R⁹ are each hydrogen and R⁷ is alkyl (e.g., ethyl).

In another embodiment, A, B, D, W, X, Y and Z are each carbon, E is nitrogen, R¹ is hydroxy, R², R⁴, R⁵, R⁶, R⁷, R⁸, R¹⁰, R¹¹ and R¹² are hydrogen, R³ is nitro, R⁹ is absent and R¹³ is aryl, such as halogen substituted phenyl (e.g., 4-fluorophenyl or 2,4-fluorophenyl).

In a further embodiment, B, D, E, W, X, Y and Z are each carbon, A is nitrogen, R¹ is hydroxy, R², R⁴, R⁵, R⁷, R⁸, R⁹, R¹⁰, R¹¹ and R¹² are hydrogen, R⁶ is absent, R³ is nitro and R¹³ is aryl, such as halogen substituted phenyl (e.g., 4-fluorophenyl or 2,4-fluorophenyl).

In yet another embodiment, A, B, D, E, X, Y and Z are each carbon, W is nitrogen, R¹ is hydroxy, R², R⁴, R⁷, R⁸, R⁹, R¹⁰, R¹¹ and R¹² are each hydrogen, R³ is nitro, R⁵ is absent, R⁶ is halogen (e.g., fluorine) and R¹³ is aryl, such as halogen substituted phenyl (e.g., 4-fluorophenyl).

In one embodiment, A, B, D, E, X, W, and Z are each carbon, Y is nitrogen, R¹ is hydroxy, R², R⁴, R⁵, R⁶, R⁷, R⁸, R⁹, R¹⁰, R¹¹ and R¹² are each hydrogen, R¹² is hydroxyl and R¹³ is aryl, such as halogen substituted phenyl (e.g., 4-fluorophenyl).

In another embodiment, A, B, D, E, X, Y and Z are each carbon, W is nitrogen, R¹ is hydroxy, R², R³, R⁴, R⁶, R⁷, R⁸, R⁹, R¹⁰, R¹¹ and R¹² are each hydrogen, R⁵ is hydroxy and R¹³ is aryl, such as halogen substituted phenyl (e.g., 4-fluorophenyl).

In a further embodiment, A, B, D, E, W, X and Z are each carbon, Y is nitrogen, R¹ is hydroxyl, R², R⁴, R⁵, R⁶, R⁷, R⁸, R⁹, R¹⁰, R¹¹ and R¹² are each hydrogen, R³ is absent and R¹³ is aryl (e.g., substituted phenyl, such as 4-fluorophenyl).

In one embodiment, the invention pertains, at least in part, to a method for reducing antibiotic resistance of a microbial cell, comprising contacting said cell with a transcription factor modulating compound of the formula (II):

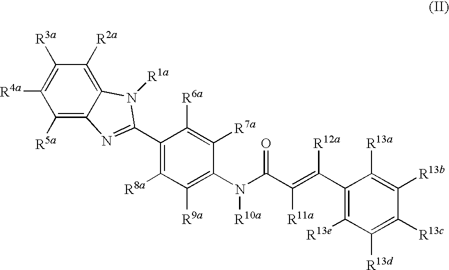

wherein

R^{1a} is hydroxyl, OCOCO₂H, a straight or branched C₁-C₅ alkyloxy group, or a straight or branched C₁-C₅ alkyl group;

R^{2a}, R^{3a}, R^{4a}, R^{5a}, R^{6a}, R^{7a}, R^{8a}, R^{9a}, R^{10a}, R^{11a}, R^{12a}, R^{13a}, R^{13b}, R^{13c}, R^{13d} and R^{13e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof;

provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b}, R^{13d} and R^{13e} are hydrogen, then R^{13c} is not hydrogen, fluorine, dimethylamino, cyano, hydroxy, methyl or methoxy; and

provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b} and R^{13d} are hydrogen, then R^{13c} and R^{13e} are not fluorine; such that the antibiotic resistance of said microbial cell is reduced.

In yet another embodiment, the invention pertains, at least in part, to a method for modulating transcription, comprising contacting a transcription factor with a transcription factor modulating compound of the formula (II):

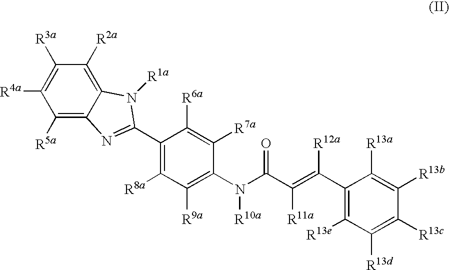

wherein

R^{1a} is hydroxyl, OCOCO₂H, a straight or branched C₁-C₅ alkyloxy group, or a straight or branched C₁-C₅ alkyl group;

R^{2a}, R^{3a}, R^{4a}, R^{5a}, R^{6a}, R^{7a}, R^{8a}, R^{9a}, R^{10a}, R^{11a}, R^{12a}, R^{13a}, R^{13b}, R^{13c}, R^{13d} and R^{13e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof;

provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b}, R^{13d} and R^{13e} are hydrogen, then R^{13c} is not hydrogen, fluorine, dimethylamino, cyano, hydroxy, methyl or methoxy; and

provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b} and R^{13d} are hydrogen, then R^{13c} and R^{13e} are not fluorine; such that transcription is modulated.

In one embodiment, R^{1a} is hydroxy and R^{3a} is cyano and R^{2a}, R^{4a}, R^{5a}, R^{6a}, R^{7a}, R^{8a}, R^{9a}, R^{10a}, R^{11a}, R^{12a}, R^{13a}, R^{13b}, R^{13c}, R^{13d} and R^{13e} are each hydrogen

In another embodiment, R^{1a} is hydroxyl, R^{3a} is cyano, R^{2a}, R^{4a}, R^{5a}, R^{6a}, R^{7a}, R^{8a}, R^{9a}, R^{10a}, R^{11a}, R^{12a}, R^{13a}, R^{13b}, R^{13d} and R^{13e} are each hydrogen and R^{13c} is halogen (e.g., fluorine), alkyl (e.g., methyl) or acyl.

In yet another embodiment, R^{1a} is hydroxy and R^{3a} is nitro, R^{2a}, R^{4a}, R^{5a}, R^{6a}, R^{7a}, R^{8a}, R^{9a}, R^{10a}, R^{12a}, R^{13a}, R^{13b}, R^{13c}, R^{13d} and R^{13e} are each hydrogen and R^{11a} is aryl (e.g., phenyl), halogen (e.g., fluorine) or alkyl (e.g., methyl).

In another embodiment, R^{1a} is hydroxyl, R^{3a} is nitro, R^{2a}, R^{2b}, R^{4a}, R^{5a}, R^{6a}, R^{7a} R^{8a}, R^{9a}, R^{10a}, R^{12a}, R^{13a}, R^{13b}, R^{13d}, and R^{13e} are each hydrogen, R^{13c} is halogen (e.g., fluorine) and R^{11a} is alkyl (e.g., hydroxyethyl or piperazinylmethyl).

In a further embodiment, R^{1a} is hydroxyl, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b}, R^{13d} and R^{13e} are each hydrogen and R^{13c} is alkyl (e.g., isopropyl), acyl or heteroaryl (e.g., triazole, imidazole or oxazole).

In one embodiment, R^{1a} is hydroxy and R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b} and R^{13d} are each hydrogen and R^{13c} and R^{13e} are each alkoxy (e.g., methoxy).

In another embodiment, R^{1a} is hydroxy and R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13d} and R^{13e} are each hydrogen and R^{13b} is alkyl (e.g., alkyl substituted with phosphonic acid or phosphonic acid dialkyl ester) and R^{13e} is halogen (e.g., fluorine).

In one embodiment, R^{1a} is hydroxyl, R^{3a} is nitro, R^{13c} is halogen (e.g., fluorine), R^{2a}, R^{5a}, R^{6a}, R^{7a}, R^{8a}, R^{9a}, R^{10a}, R^{11a}, R^{12a}, R^{13a}, R^{13b}, R^{13d} and R^{13e} are each hydrogen and R^{4a} is alkylamino (e.g., dimethylamino or dialkylaminoalkylamino), alkyl (e.g., methyl) or alkoxy (e.g., ethoxy, phosphonic acid substituted alkoxy, ether substituted alkoxy, alkylamino substituted alkoxy, or heterocyclic substituted alkoxy, for example, morpholine substituted alkoxy or piperazine substituted alkoxy) or halogen (e.g., fluorine)

In yet another embodiment, R^{1a} is hydroxyl, R^{3a} is nitro, R^{13c} is halogen (e.g., R^{4a}, R^{5a}, R^{6a}, R^{7a}, R^{8a}, R^{9a}, R^{10a}, R^{11a}, R^{12a}, R^{13a}, R^{13b}, R^{13d} and R^{13e} each hydrogen and R^{2a} is alkylamino (e.g., alkylaminoalkylamino, such as dimethylaminoethylamino).

In a further embodiment, R^{1a} is a substituted or unsubstituted straight or branched C₁-C₅ alkyloxy group (e.g., phosphonic acid substituted alkoxy or phosphonic acid dialkyl ester alkoxy), R^{3a} is nitro, R^{13c} is halogen (e.g., fluorine), R^{2a}, R^{4a}, R^{5a}, R^{6a}, R^{7a}, R^{8a}, R^{9a}, R^{10a}, R^{11a}, R^{12a}, R^{13a}, R^{13b}, R^{13d} and R^{13e} are each hydrogen.

In yet another embodiment, R^{1a} is hydroxyl, R^{3a} is nitro, R^{2a}, R^{5a}, R^{6a}, R^{7a}, R^{8a}, R^{9a}, R^{10a}, R^{11a}, R^{12a}, R^{13a}, R^{13b}, R^{13d} and R^{13e} are hydrogen, R^{13c} is acyl and R^{4a} is alkoxy (e.g., piperazinyl substituted alkoxy or morpholine substituted alkoxy).

In a further embodiment, R^{1a} is hydroxyl, R^{3a} is heteroaryl (e.g., imidazolyl or pyrazolyl), R^{3a}, R^{4a}, R^{5a}, R^{6a}, R^{7a}, R^{8a}, R^{9a}, R^{10a}, R^{11a}, R^{12a}, R^{13a}, R^{13b}, R^{13d} and R^{13e} are each hydrogen, and R^{13c} is halogen (e.g., fluorine).

In another embodiment, the invention pertains, at least in part, to a method for reducing antibiotic resistance of a microbial cell, comprising contacting said cell with a transcription factor modulating compound of the formula (III):

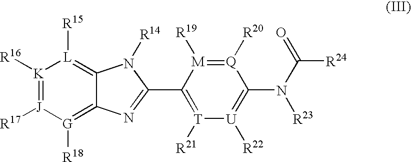

wherein

R¹⁴ is hydroxyl, OCOCO₂H, a straight or branched C₁-C₅ alkyloxy group, or a straight or branched C₁-C₅ alkyl group;

G, J, K, L, M, Q, T and U are each independently carbon or nitrogen;

wherein R¹⁵, R¹⁶, R¹⁷, R¹⁸, R¹⁹, R²⁰, R²¹, R²², R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen, when G, J, K, L, M, Q, T and U are carbon; or R¹⁵, R¹⁶, R¹⁷, R¹⁸, R¹⁹, R²⁰, R²¹, R²², R²³ and R²⁴ are each independently absent or hydroxyl when G, J, K, L, M, Q, T and U are nitrogen;

R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen; and pharmaceutically acceptable salts, esters and prodrugs thereof;

provided that when G, J, K, L, M, Q, T and U are each carbon, one of R¹⁵, R¹⁶, R¹⁷, R¹⁸, R¹⁹, R²⁰, R²¹, R²², R²³ and R²⁴, are not hydrogen, such that the antibiotic resistance of said microbial cell is reduced.

In yet another embodiment, the invention pertains, at least in part, to a method for modulating transcription, comprising contacting a transcription factor with a transcription factor modulating compound of the formula (III):

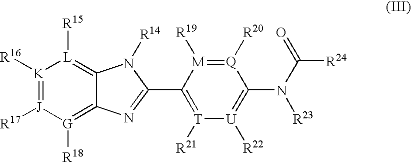

wherein

R¹⁴ is hydroxyl, OCOCO₂H, a straight or branched C₁-C₅ alkyloxy group, or a straight or branched C₁-C₅ alkyl group;

G, J, K, L, M, Q, T and U are each independently carbon or nitrogen;

wherein R¹⁵, R¹⁶, R¹⁷, R¹⁸, R¹⁹, R²⁰, R²¹, R²², R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen, when G, J, K, L, M, Q, T and U are carbon; or R¹⁵, R¹⁶, R¹⁷, R¹⁸, R¹⁹, R²⁰, R²¹, R²², R²³ and R²⁴ are each independently absent or hydroxyl when G, J, K, L, M, Q, T and U are nitrogen;

R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen; and pharmaceutically acceptable salts, esters and prodrugs thereof;

provided that when G, J, K, L, M, Q, T and U are each carbon, one of R¹⁵, R¹⁶, R¹⁷, R¹⁸, R¹⁹, R²⁰, R²¹, R²², R²³ and R²⁴, are not hydrogen, such that transcription is modulated.

In one embodiment, G, J, K, L, M, Q, T and U are each carbon, R¹⁴ is hydroxy, R¹⁶ is nitro, R²⁴ is aryl (e.g., phenyl, such as acyl substituted phenyl), R¹⁵, R¹⁷, R¹⁸, R¹⁹, R²⁰ and R²¹ are hydrogen and R²² is halogen (e.g., fluorine).

In another embodiment, G, J, K, L, M, Q, T and U are each carbon, R¹⁴ is hydroxy, R¹⁶ is nitro, R²⁴ is aryl (e.g., phenyl, such as acyl substituted phenyl), R¹⁵, R¹⁷, R¹⁸, R¹⁹, R²¹ and R²² are hydrogen and R²⁰ is alkyl (e.g., methyl or ethyl).

In yet another embodiment, G, J, K, L, M, Q, T and U are each carbon, R¹⁴ is hydroxy, R¹⁶ is nitro, R²⁴ is aryl (e.g., phenyl, such as acyl substituted phenyl), R¹⁵, R¹⁷, R¹⁸, R¹⁹, R²⁰ and R²² are hydrogen and R²¹ is alkoxy (e.g., methoxy).

In a further embodiment, G, J, K, L, M, Q, T and U are each carbon, R¹⁴ is hydroxy, R¹⁶ is nitro, R²⁴ is aryl (e.g., phenyl, such as halogen substituted phenyl, for example, 4-fluorophenyl), R¹⁵, R¹⁷, R¹⁸, R¹⁹, R²⁰ and R²² are hydrogen and R²¹ is halogen (e.g., fluorine) or alkoxy (e.g., methoxy or phosphonic acid substituted alkoxy).

In one embodiment, G, J, K, L, M, Q, T and U are each carbon, R¹⁴ is hydroxy, R¹⁶ is nitro, R²⁴ is aryl (e.g., phenyl, such as halogen substituted phenyl, for example, 4-fluorophenyl), R¹⁵, R¹⁷, R¹⁸, R¹⁹, R²¹ and R²² are hydrogen and R²⁰ is alkyl (e.g., ethyl).

In one embodiment, G, J, K, L, Q, T and U are each carbon, M is nitrogen, R¹⁴ is hydroxy, R¹⁶ is nitro, R¹⁵, R¹⁷, R¹⁸, R²⁰, R²¹, R²² and R²³ are each hydrogen, R¹⁹ is absent and R²⁴ is aryl, such as, for example, substituted phenyl, and in particular, halogen substituted phenyl (e.g., 4-fluorophenyl) or acyl substituted phenyl (e.g., 4-acyl substituted phenyl).

In another embodiment, G, J, K, L, M, Q and T are each carbon, U is nitrogen, R¹⁴ is hydroxy, R¹⁶ is nitro, R¹⁵, R¹⁷, R¹⁸, R¹⁹, R²⁰, R²¹, and R²³ are each hydrogen, R²² is absent and R²⁴ is aryl, such as, for example, phenyl such as halogen substituted phenyl (4-fluorophenyl).

In yet another embodiment, wherein J, K, L, M, Q, T and U are each carbon, G is nitrogen, R¹⁴ is hydroxy, R¹⁶ is nitro, R¹⁵, R¹⁷, R¹⁹, R²⁰, R²¹, R²² and R²³ are each hydrogen, R¹⁸ is absent and R²⁴ is aryl, such as, for example, phenyl, which may be substituted with halogen (e.g., 4-fluorophenyl) or acyl (e.g., 4-acylphenyl).

In one embodiment, G, J, L, M, Q, T and U are each carbon, K is nitrogen, R¹⁴ is hydroxy, R¹⁶ is absent, R¹⁵, R¹⁷, R¹⁸, R¹⁹, R²⁰, R²¹, R²² and R²³ are each hydrogen and R²⁴ is aryl, such as, for example, phenyl, which may be substituted with halogen (e.g., 4-fluorophenyl).

In one embodiment, the invention pertains, at least in part, to a method for reducing antibiotic resistance of a microbial cell, comprising contacting said cell with a transcription factor modulating compound of the formula (IV):

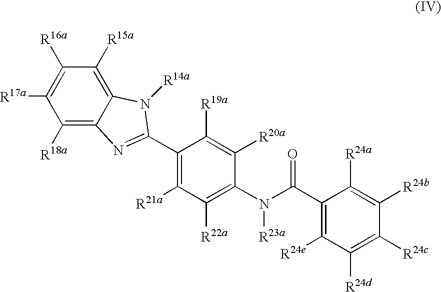

wherein

R^{14a} is hydroxyl, OCOCO₂H, a straight or branched C₁-C₅ alkyloxy group, or a straight or branched C₁-C₅ alkyl group;

R^{15a}, R^{16a}, R^{17a}, R^{18a}, R^{19a}, R^{20a}, R^{21a}, R^{22a}, R^{23a} and R^{24a}, R^{24b}, R^{24c}, R^{24d} and R^{24e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof;

provided that at least two of R^{24a}, R^{24b}, R^{24c}, R^{24d} and R^{24e} are not hydrogen, such that the antibiotic resistance of said microbial cell is reduced.

In another embodiment, the invention pertains, at least in part, to a method for modulating transcription, comprising contacting a transcription factor with a transcription factor modulating compound of the formula (IV):

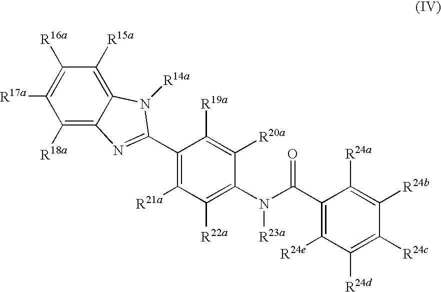

wherein

R^{14a} is hydroxyl, OCOCO₂H, a straight or branched C₁-C₅ alkyloxy group, or a straight or branched C₁-C₅ alkyl group;

R^{15a}, R^{16a}, R^{17a}, R^{18a}, R^{19a}, R^{20a}, R^{21a}, R^{22a}, R^{23a} and R^{24a}, R^{24b}, R^{24c}, R^{24d} and R^{24e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; or R^{24c} and R^{24d} are connected to form a ring; and esters, prodrugs and pharmaceutically acceptable salts thereof;

provided that at least two of R^{24a}, R^{24b}, R^{24c}, R^{24d} and R^{24e} are not hydrogen, such that transcription is modulated.

In one embodiment, R^{14a} is hydroxyl, R^{15a}, R^{17a}, R^{18a}, R^{19a}, R^{20a}, R^{21a}, R^{22a}, R^{23a}, R^{24a}, R^{24b} and R^{24e} are hydrogen, R^{16a} is nitro and R^{24c} and R^{24d} are joined to form a ring (e.g., a six membered ring, such as cyclohexanone).

In another embodiment, R^{14a} is hydroxyl, R^{15a}, R^{17a}, R^{18a}, R^{19a}, R^{20a}, R^{21a}, R^{22a}, R^{23a}, R^{24a}, R^{24b} and R^{24e} are hydrogen, R^{16a} is nitro and R^{24c} is halogen (e.g., fluorine) and R^{24d} is halogen (e.g., fluorine), alkyl (e.g., methyl) or alkoxy (e.g., methoxy).

In yet another embodiment, R^{14a} is hydroxyl, R^{15a}, R^{17a}, R^{18a}, R^{19a}, R^{20a}, R^{21a}, R^{22a}, R^{23a}, R^{24a}, R^{24b} and R^{24d} are hydrogen, R^{16a} is nitro, R^{24c} is halogen (e.g., fluorine) and R^{24e} is alkoxy (e.g., methoxy).

In a further embodiment, the invention pertains, at least in part, to a method for reducing antibiotic resistance of a microbial cell, comprising contacting said cell with a transcription factor modulating compound of the formula (V):

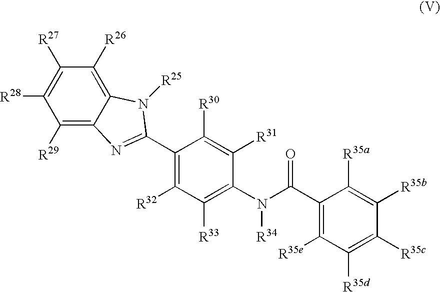

wherein

R²⁵ is hydroxyl, OCOCO₂H, a straight or branched C₁-C₅ alkyloxy group, or a straight or branched C₁-C₅ alkyl group;

R²⁶, R²⁷, R²⁸, R²⁹, R³⁰, R³¹, R³², R³³, R³⁴, R^{35a}, R^{35b}, R^{35c}, R^{35d}, and R^{35e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof;

provided that at least two of R²⁶, R²⁷, R²⁸ and R²⁹ are not hydrogen, such that the antibiotic resistance of said microbial cell is reduced.

In another embodiment, the invention pertains to a method for modulating transcription, comprising contacting a transcription factor with a transcription factor modulating compound of the formula (V):

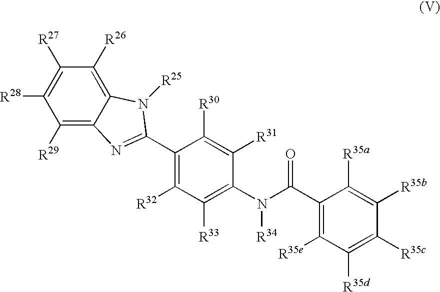

wherein

R²⁵ is hydroxyl, OCOCO₂H, a straight or branched C₁-C₅ alkyloxy group, or a straight or branched C₁-C₅ alkyl group;

R²⁶, R²⁷, R²⁸, R²⁹, R³⁰, R³¹, R³², R³³, R³⁴, R^{35a}, R^{35b}, R^{35c}, R^{35d}, and R^{35e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof;

provided that at least two of R²⁶, R²⁷, R²⁸ and R²⁹ are not hydrogen, such that transcription is modulated.

In one embodiment, R²⁵ is hydroxy, R²⁶, R²⁹, R³⁰, R³¹, R³², R³³, R³⁴, R^{35a}, R^{35b}, R^{35d}, and R^{35e} are each hydrogen, R²⁷ is nitro, R²⁸ is alkyl (e.g., methyl) and R^{35c} is acyl or heteroaryl (e.g., oxazole).

In one embodiment, the invention pertains to a method for reducing antibiotic resistance of a microbial cell, comprising contacting said cell with a transcription factor modulating compound of the formula (VI):

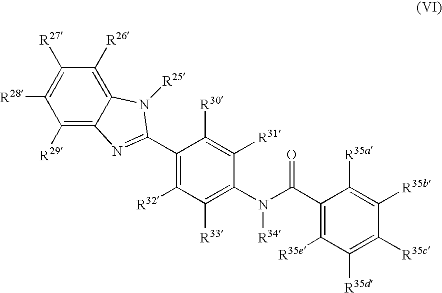

wherein

R^{25′} is a substituted straight or branched C₁-C₅ alkyloxy group;

R^{26′}, R^{27′}, R^{28′}, R^{29′}, R^{30′}, R^{31′}, R^{32′}, R^{33′}, R^{34′}, R^{35a′}, R^{35b′}, R^{35c′}, R^{35d′}, and R^{35e′} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof; such that the antibiotic resistance of said microbial cell is reduced.

In another embodiment, the invention pertains to a method for modulating transcription, comprising contacting a transcription factor with a transcription factor modulating compound of the formula (VI):

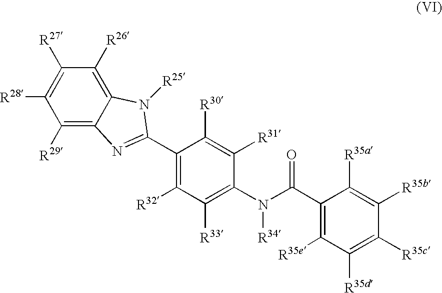

wherein

R^{25′} is substituted straight or branched C₁-C₅ alkoxy group;

R^{26′}, R^{27′}, R^{28′}, R^{29′}, R^{30′}, R^{31′}, R^{32′}, R^{33′}, R^{34′}, R^{35a′}, R^{35b′}, R^{35c′}, R^{35d′}, and R^{35e′} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen; and esters, prodrugs and pharmaceutically acceptable salts thereof; such that transcription is modulated.

In one embodiment, R^{26′}, R^{28′}, R^{29′}, R^{30′}, R^{31′}, R^{32′}, R^{33′}, R^{34′}, R^{35a′}, R^{35b′}, R^{35d′} and R^{35e′} are each hydrogen, R^{27′} is nitro, R^{35c′} is halogen (e.g., fluorine) and R^{25′} phosphonic acid substituted alkoxy, alkyl phosphonic acid substituted alkoxy, carboxylic acid substituted alkoxy or alkylamino substituted alkoxy.

In another embodiment, the present invention, pertains, at least in part, to a method for reducing antibiotic resistance of a microbial cell, comprising contacting said cell with a transcription factor modulating compound of the formula (VII):

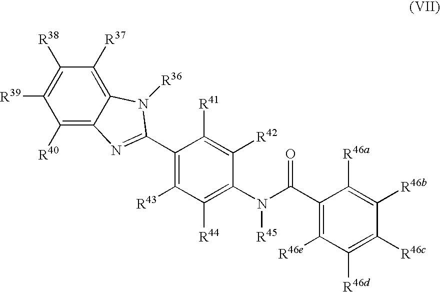

wherein

R³⁶ is hydroxyl;

R³⁷, R³⁹, R⁴⁰, R⁴¹, R⁴², R⁴³, R⁴⁴, R⁴⁵, R^{46a}, R^{46b}, R^{46d} and R^{46e} are each independently hydrogen, alkyl alkenyl, alkynyl, aryl, heteroaryl, heterocyclyl, alkoxy, aryloxy, heteroaryloxy, alkoxycarbonyl aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;

R³⁸ is cyano, nitro, oxime, alkyloxime, aryloxime, heteroaryl, amino-oxime, or aminocarbonyl;

R^{46c} is hydrogen, acyl, fluoro, pyrizinyl, pyridinyl, cyano, imidazolyl, dialkylaminocarbonyl or dialkylamino; and esters, prodrugs and pharmaceutically acceptable salts thereof;

provided that when R³⁸ is nitro and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino, acyl or hydrogen; and

provided that when R³⁸ is cyano and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino; such that the antibiotic resistance of said microbial cell is reduced.

In a further embodiment, the present invention pertains, at least in part, to a method for modulating transcription, comprising contacting a transcription factor with a transcription factor modulating compound of the formula (VII):

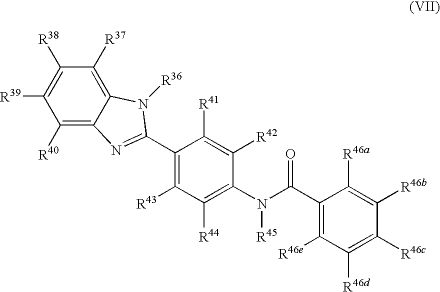

wherein

R³⁶ is hydroxyl;

R³⁷, R³⁹, R⁴⁰, R⁴¹, R⁴², R⁴³, R⁴⁴, R⁴⁵, R^{46a}, R^{46b}, R^{46d}, and R^{46e} are each independently hydrogen, alkyl alkenyl, alkynyl, aryl, heteroaryl, heterocyclyl, alkoxy, aryloxy, heteroaryloxy, alkoxycarbonyl aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;

R³⁸ is cyano, nitro, oxime, alkyloxime, aryloxime, heteroaryl, amino-oxime, or aminocarbonyl;

R^{46c} is hydrogen, acyl, fluoro, pyrizinyl, pyridinyl, cyano, imidazolyl, dialkylaminocarbonyl or dialkylamino; and esters, prodrugs and pharmaceutically acceptable salts thereof;

provided that when R³⁸ is nitro and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino, acyl or hydrogen; and

provided that when R³⁸ is cyano and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino; such that transcription is modulated.

In one embodiment, R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, and R³⁸ is cyano and R^{46c} is acyl, fluoro, cyano or imidazolyl.

In another embodiment, R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, and R³⁸ is amino-oxime and R^{46c} is fluoro.

In a further embodiment, R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, and R³⁸ is nitro and R^{46c} is pyrizinyl, pyridinyl or dialkylaminocarbonyl (e.g., dimethylaminocarbonyl).

In another embodiment, R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, and R³⁸ is aminocarbonyl and R^{46c} is halogen (e.g., fluorine).

In one embodiment, R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, and R³⁸ is oxime and R^{46c} is dialkylamino (e.g., dimethylamino).

In another embodiment, R³⁷, R³⁹, R⁴⁰, R⁴¹, R⁴², R⁴³, R⁴⁴, R⁴⁵, R^{46b}, R^{46c}, R^{46d}, and R^{46e} are each hydrogen, and R³⁸ is nitro and R^{46a} is hydroxyl.

In another embodiment, R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, and R³⁸ is heteroaryl (e.g., imidazolyl or pyrazolyl) and R^{46c} is acyl.

In a further embodiment, the present invention pertains, at least in part, to a method for reducing antibiotic resistance of a microbial cell, comprising contacting said cell with a transcription factor modulating compound of the formula (VIII):

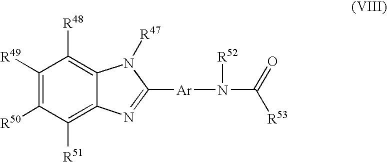

wherein

R⁴⁷ is hydroxyl, OCOCO₂H, a straight or branched C₁-C₅ alkyloxy group, or a straight or branched C₁-C₅ alkyl group;

R⁴⁸, R⁴⁹, R⁵⁰, R⁵¹, R⁵² and R⁵³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;

Ar is aryl; and pharmaceutically acceptable salts, esters and prodrugs thereof; such that the antibiotic resistance of said microbial cell is reduced.

In one embodiment, the present invention pertains, at least in part, to a method for modulating transcription, comprising contacting a transcription factor with a transcription factor modulating compound of the formula (VIII):

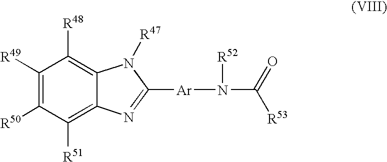

wherein

R⁴⁷ is hydroxyl, OCOCO₂H, a straight or branched C₁-C₅ alkyloxy group, or a straight or branched C₁-C₅ alkyl group;

R⁴⁸, R⁴⁹, R⁵⁰, R⁵¹, R⁵² and R⁵³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;

Ar is aryl; and pharmaceutically acceptable salts, esters and prodrugs thereof; such that transcription is modulated.

In one embodiment, R⁴⁷ is hydroxy, R⁴⁸, R⁵⁰, R⁵¹ and R⁵² are each hydrogen, Ar is furanyl, and R⁵³ is alkenyl, which may be substituted with phenyl, such as, for example, halogen substituted phenyl (e.g., fluorophenyl).

In another embodiment, the invention pertains to inhibiting transcription, comprising contacting a transcription factor with a transcription factor modulating compound, such that transcription is inhibited. In a further embodiment, the transcription of a prokaryotic cell is inhibited. In another further embodiment, the transcription factor modulating compound is a compound of anyone of formulae (I)-(VIII) and of Table 2.

The term “antibiotic resistance” includes resistance of a microbial cell to a antibiotic compound, especially an antibiotic compound which had been previously used to treat similar microbial organisms successfully.

In one embodiment, the transcription factor modulating compound (e.g., MarA family polypeptide modulating compound, AraC family polypeptide modulating compound, etc.) is of anyone of formulae (I)-(VIII) and of Table 2.

In a further embodiment, the transcription factor modulating compound is:

TABLE 2

| Code | Compound |
|---|---|
| A | 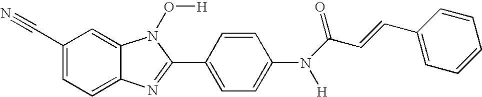 |
| B | 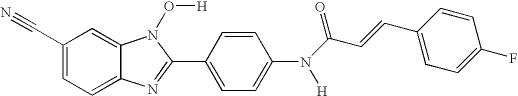 |
| C | 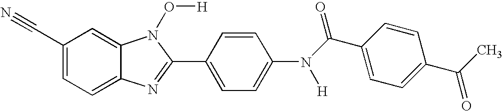 |
| D | 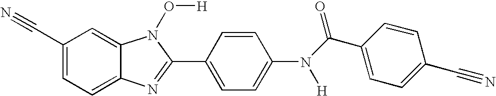 |
| E | 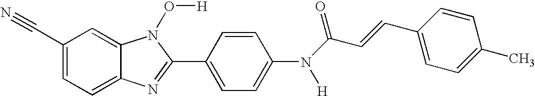 |
| F | 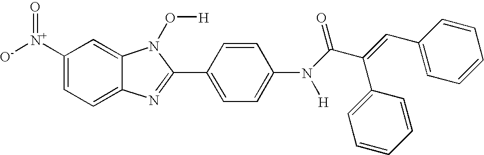 |
| G | 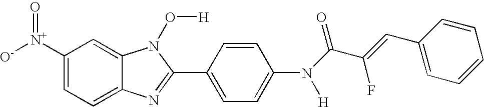 |
| H | 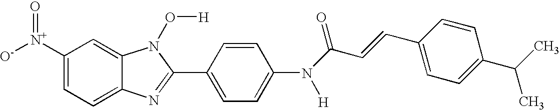 |
| I | 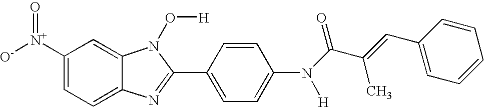 |
| J | 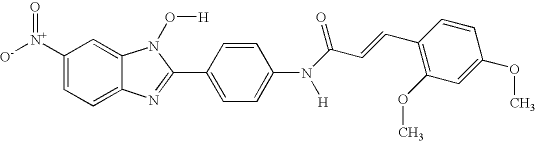 |
| K | 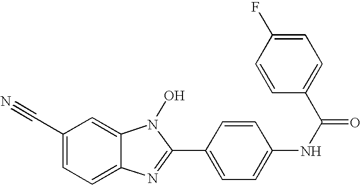 |
| L | 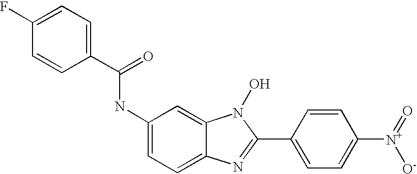 |
| M | 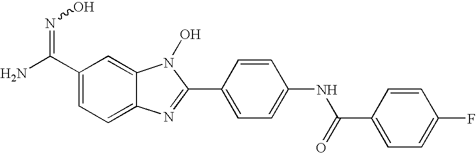 |
| N | 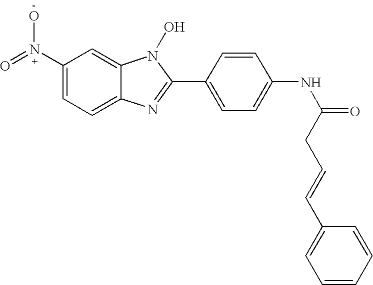 |
| O | 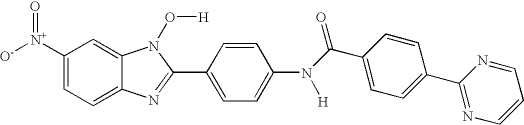 |
| P | 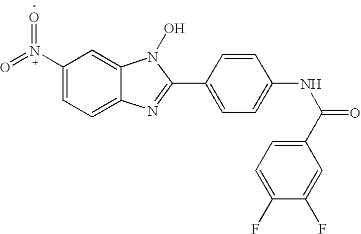 |
| Q | 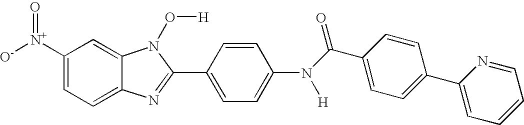 |
| R | 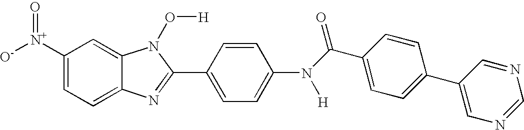 |
| S | 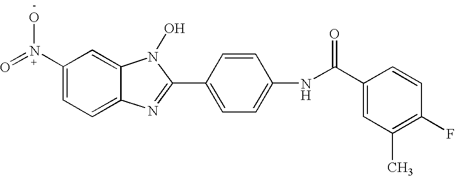 |
| T | 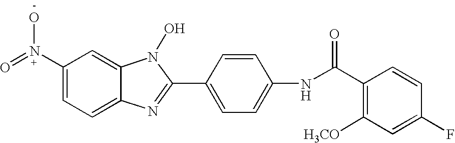 |
| U | 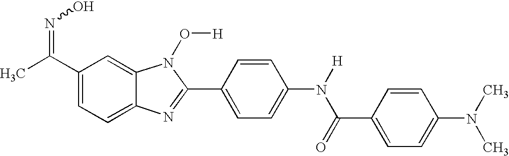 |
| V | 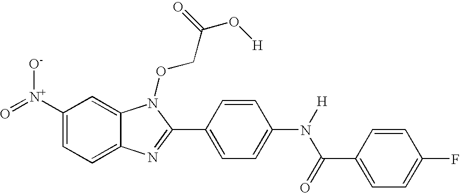 |
| W | 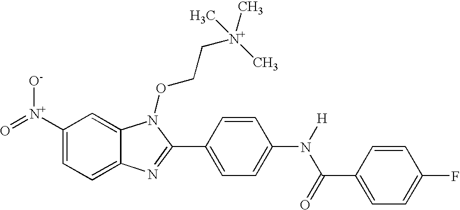 |
| X | 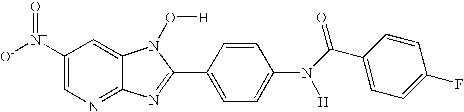 |
| Y | 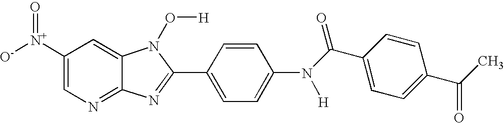 |
| Z | 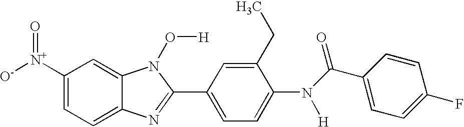 |
| AA | 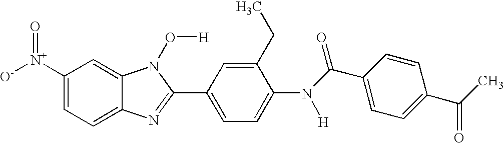 |
| AB | 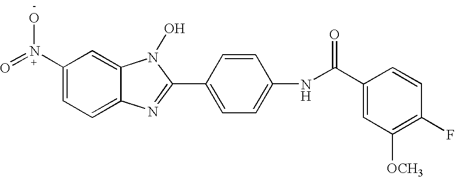 |
| AC | 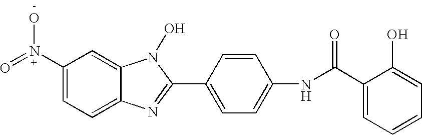 |
| AD | 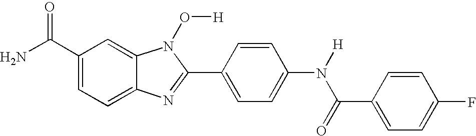 |
| AE | 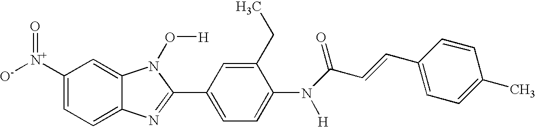 |
| AF | 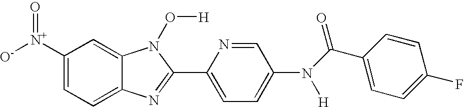 |
| AG | 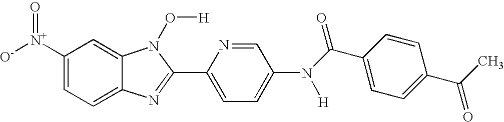 |
| AH | 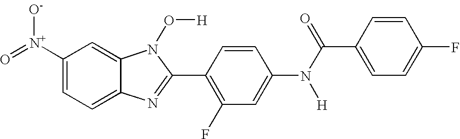 |
| AI | 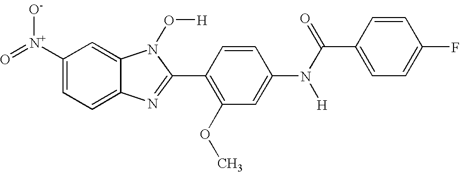 |
| AJ | 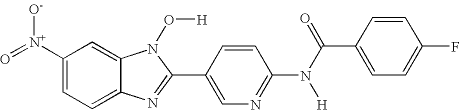 |
| AK | 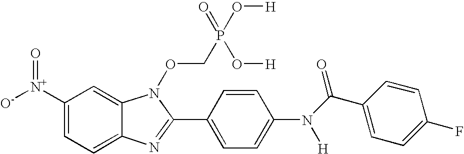 |
| AL | 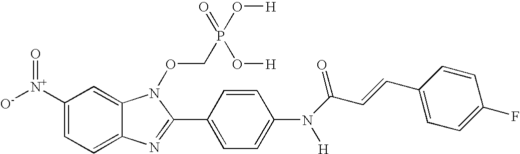 |
| AM | 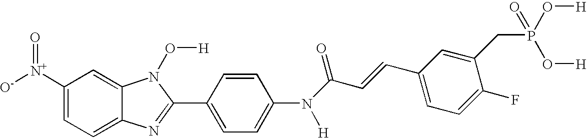 |
| AN | 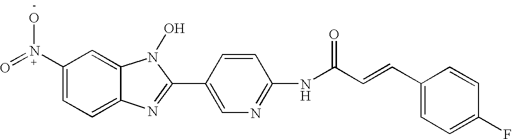 |
| AO | 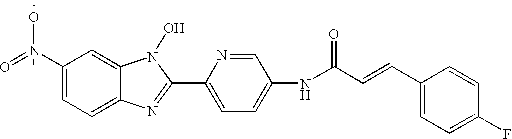 |
| AP | 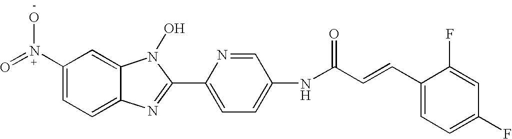 |
| AQ | 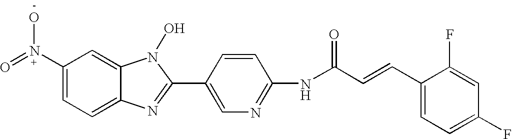 |
| AR | 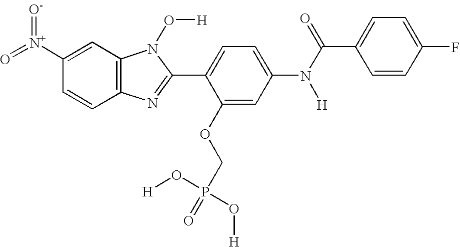 |
| AS | 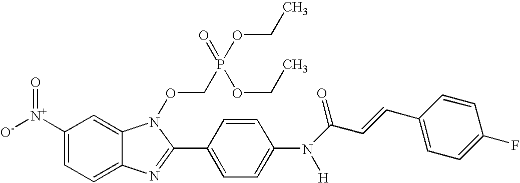 |
| AT | 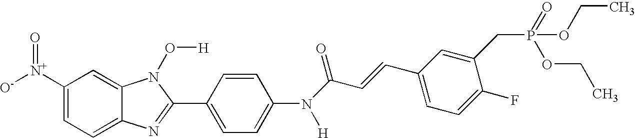 |
| AU | 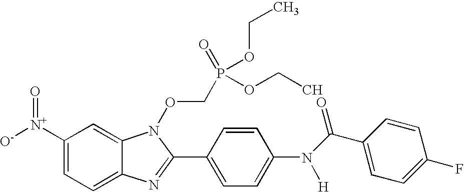 |
| AV | 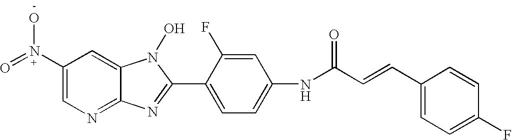 |
| AW | 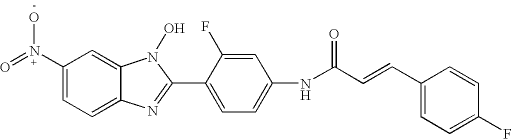 |
| AX | 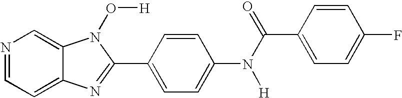 |
| AY | 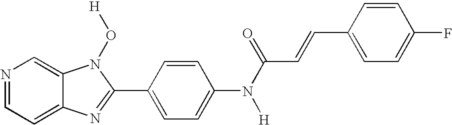 |
| AZ | 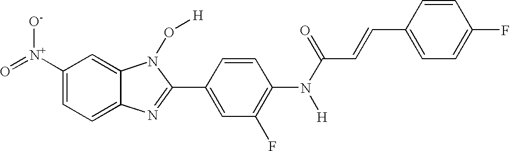 |
| BA | 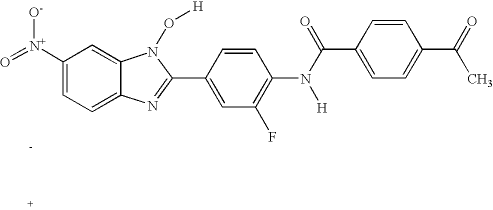 |
| BB | 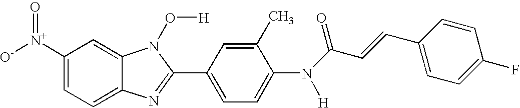 |
| BC | 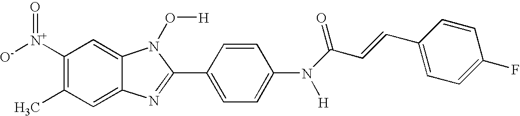 |
| BD | 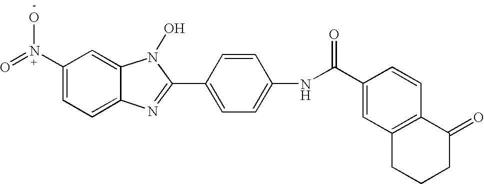 |
| BE | 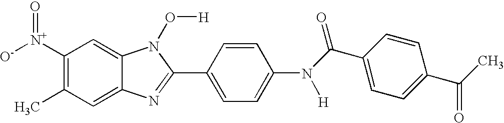 |
| BF | 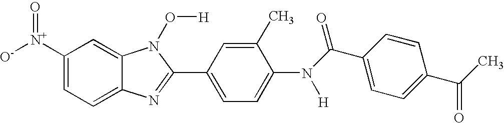 |
| BG | 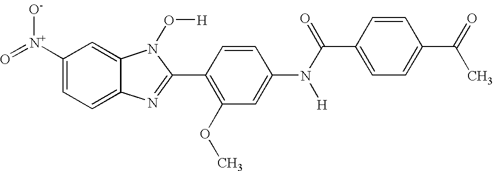 |
| BH | 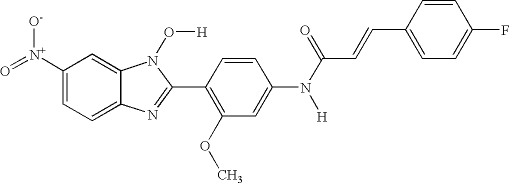 |
| BI | 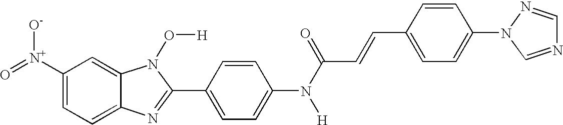 |
| BJ | 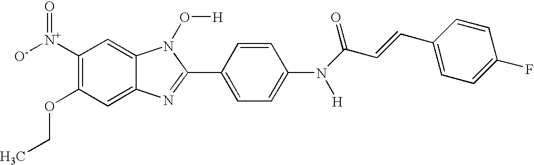 |
| BK | 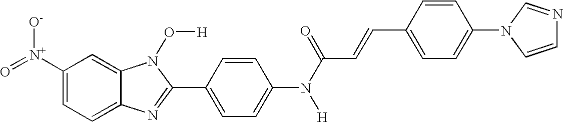 |
| BL | 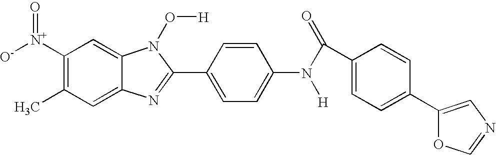 |
| BM | 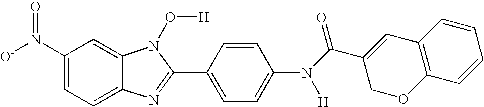 |
| BN | 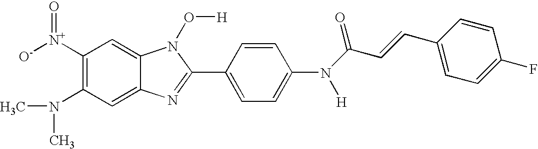 |
| BO | 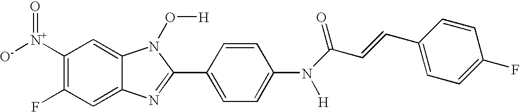 |
| BP | 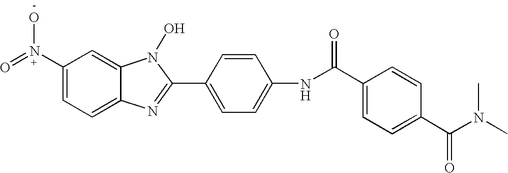 |
| BQ | 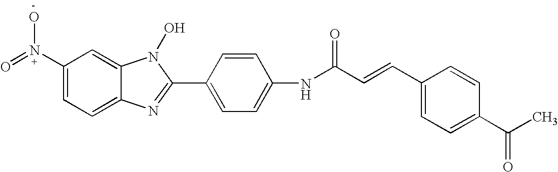 |
| BR | 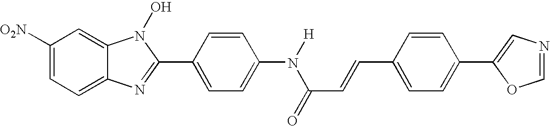 |
| BS | 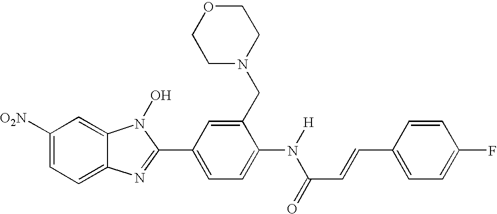 |
| BT | 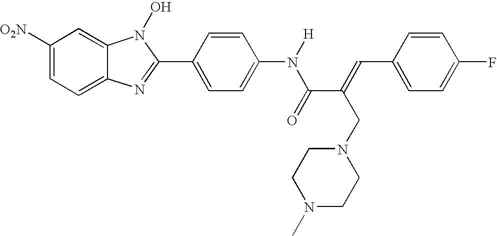 |
| BU | 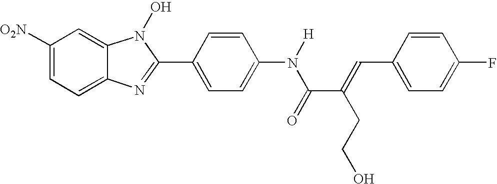 |
| BV | 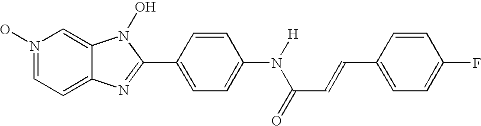 |
| BW | 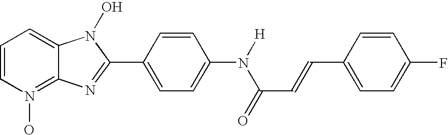 |
| BX | 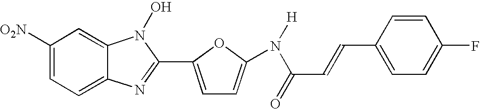 |
| BY | 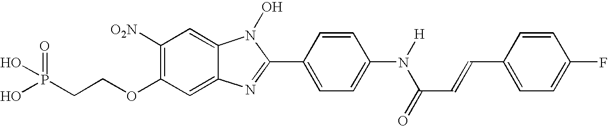 |
| BZ | 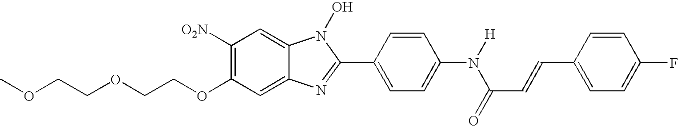 |
| CA | 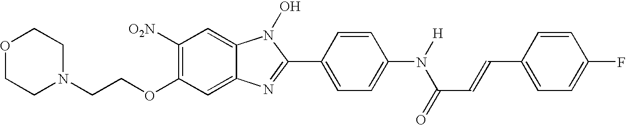 |
| CB | 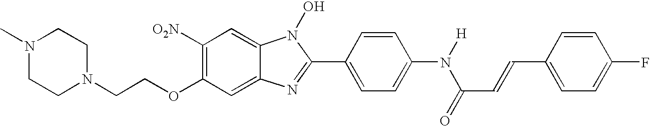 |
| CC | 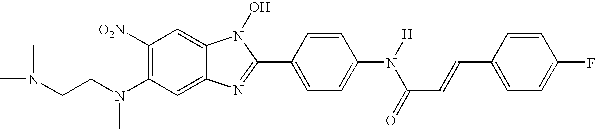 |
| CD | 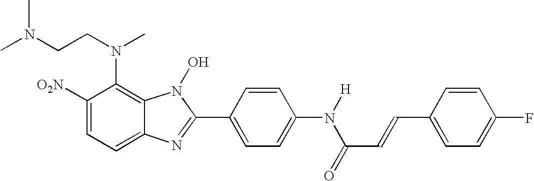 |
| CE | 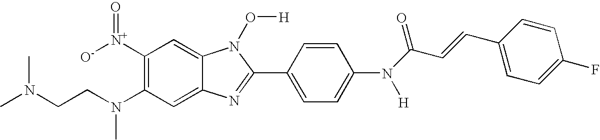 |
| CF | 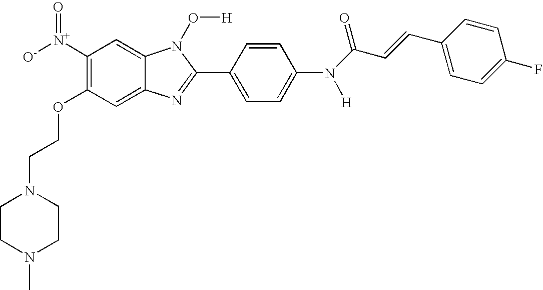 |
| CG | 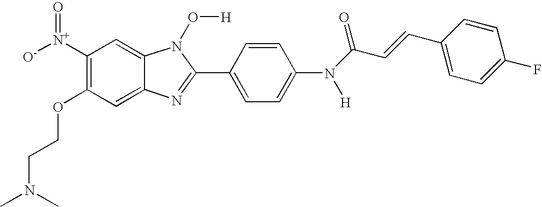 |
| CH | 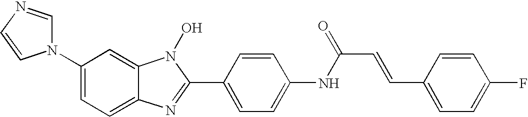 |
| CI | 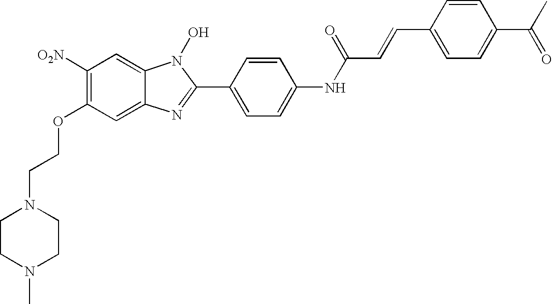 |
| CJ | 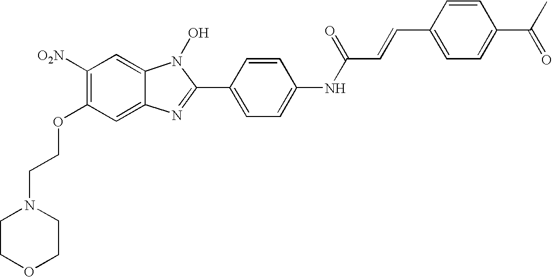 |
| CK | 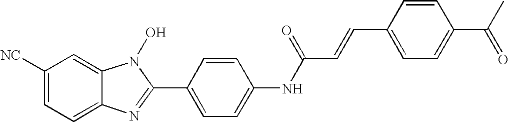 |
| CL | 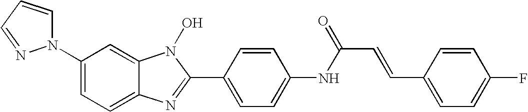 |
| CM | 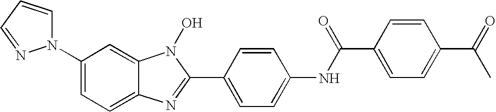 |
| CN | 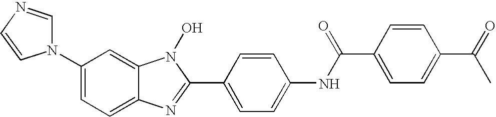 |
| CO | 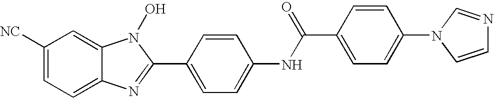 |

In one embodiment, the compounds of the invention (e.g., a compound of formulae I, II, III, IV, V, VI, VII, VIII or a compound of Table 2) are pharmaceutically acceptable salts, including, for example, a sodium salt or a potassium salt.

The EC₅₀ of a transcription factor modulating compound can be measured using the assay described in Example 12 of U.S. Ser. No. 11/115,024, incorporated herein by reference. In a further embodiment, the transcription factor modulating compound has an EC₅₀ activity against SoxS of less than about 10 μM, less than about 5 μM, or less than about 1 μM. In a further embodiment, the transcription factor modulating compound can have an EC₅₀ activity against MarA of less than about 10 μM, less than about 5 μM, or less than about 1 μM. In yet another embodiment, the transcription factor modulating compound can have an EC₅₀ against LcrF (VirF) of less than about 10 μM, less than about 5 μM, or less than about 1 μM.

In another further embodiment, the transcription factor modulating causes a log decrease in CFU/g of kidney tissue. This can be measured using the assay described Example 13 of U.S. Ser. No. 11/115,024, incorporated herein by reference. In one embodiment, the transcription factor modulating compound cause a log decrease in CFU/g of kidney tissue of greater than 1.0 CFU/g. In a further embodiment, the compound causes a log decrease in CFU/g of kidney tissue greater than 2.5 CFU/g.

In a further embodiment, the transcription factor modulating compound is not apigenin.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, the term alkyl includes both “unsubstituted alkyls” and “substituted alkyls,” the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthridine, indole, benzofuran, purine, benzofuran, deazapurine, or indolizine. Those aryl groups having heteroatoms in the ring structure may also be referred to as “aryl heterocycles,” “heterocycles,” “heteroaryls” or “heteroaromatics.” Moreover, the term heterocycle includes The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin). The term “aryl” also includes multicyclic aryl groups such as porphyrins, phthalocyanines, etc.

The term “alkenyl” includes unsaturated aliphatic groups analogous in length and possible substitution to the alkyls described above, but that contain at least one double bond.

For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls,” the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, aryl amino, diaryl amino, and alkylaryl amino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The terms “alkoxyalkyl,” “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide” or “aminocarboxy” includes compounds or moieties which contain a nitrogen atom which is bound to the carbon of a carbonyl or a thiocarbonyl group. The term includes “alkaminocarboxy” groups which include alkyl, alkenyl, or alkynyl groups bound to an amino group bound to a carboxy group. It includes arylaminocarboxy groups which include aryl or heteroaryl moieties bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, arylalkylaminocarbonyl, alkenylaminocarbonyl, alkylcarbonyl, arylcarbonyl, arylalkyl carbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

The term “electron withdrawing substituent” includes, but is not limited to, ammonium (including alkylammonium, arylammonium, and heteroarylammonium), slovenly including alkylsulfonyl, arylsulfonyl, and heteroarylsulfonyl), halogen, perhalogenated alkyl, cyano, oxime, carbonyl (including alkylcarbonyl, arylcarbonyl, and heteroarylcarbonyl), and nitro.

It will be noted that the structure of some of the compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof.

Bonds represented by “ [symbol] ” in a structural formula mean that the bond may be either a single or a double bond.

### VIII. Formulations Comprising Transcription Factor Modulating Compounds

The invention provides compositions which include a therapeutically-effective amount or dose of a transcription factor modulating compound and/or a compound identified in any of the instant assays and one or more carriers (e.g., pharmaceutically acceptable additives and/or diluents). The pharmaceutical compositions of the invention may comprise any compound described in this application as a transcription factor modulating compound, an AraC family polypeptide modulating compound, a MarA family polypeptide modulating compound, a MarA family inhibiting compound, a MarA inhibiting compound, compounds of formulae (I), (II), (III), (IV), (V), (VI), (VII), (VIII), or Table 2. Each of these compounds may be used alone of in combination as a part of a pharmaceutical composition of the invention. Furthermore, a composition can also include a second antimicrobial agent, e.g., an antibiotic.

The invention pertains to pharmaceutical compositions comprising an effective amount of a transcription factor modulating compound (e.g., a MarA family polypeptide modulating compound or an AraC family polypeptide modulating compound), and a pharmaceutically acceptable carrier. In one embodiment, the transcription factor modulating compound is of the formula (I), (II), (III), (IV), (V), (VI), (VII), (VIII) or Table 2.

In one embodiment, the present invention provides a pharmaceutical composition comprising a pharmaceutically acceptable carrier and a transcription factor modulating compound, wherein said compound is of the formula (I), (II), (III), (IV), (V), (VI), (VII), (VIII) or Table 2. In another embodiment, the pharmaceutical composition can further comprise an antibiotic. In a further embodiment, the effective amount of the pharmaceutical composition can be effective for treating a biofilm associated state in a subject. The biofilm associated states can include, for example, middle ear infections, cystic fibrosis, osteomyelitis, acne, dental cavities, endocarditis, and prostatitis.

In another embodiment, the method for preventing a bacterial associated state in a subject, comprising administering to the subject an effective amount of a transcription factor modulating compound, such that the bacterial associated state is prevented. In a further embodiment, the transcription factor modulating compound is of the formula (I), (II), (III), (IV), (V), (VI), (VII), (VIII) or a compound of Table 2. In a further embodiment, the transcription factor modulating compound can include, for example, a MarA family polypeptide inhibitor and an AraC family polypeptide inhibitor.

The term “subject” includes plants and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans) which are capable of suffering from a bacterial associated disorder. The term “subject” also comprises immunocompromised subjects, who may be at a higher risk for infection.

The term “preventing” the administration of an effective amount of the transcription factor modulating compound to prevent a bacterial associated state from occurring.

The term “bacterial associated state” includes states characterized by the presence of bacteria which can be prevented by administering the transcription factor modulating compounds of the invention. The term includes biofilm associated states and other infections or the undesirable presence of a bacteria on or in a subject.

As described in detail below, the pharmaceutical compositions can be formulated for administration in solid or liquid form, including those adapted for the following: (1) oral administration, for example, aqueous or non-aqueous solutions or suspensions, tablets, boluses, powders, granules, pastes; (2) parental administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream, foam, or suppository; or (5) aerosol, for example, as an aqueous aerosol, liposomal preparation or solid particles containing the compound.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the antiinfective agents or compounds of the invention from one organ, or portion of the body, to another organ, or portion of the body without affecting its biological effect. Each carrier should be “acceptable” in the sense of being compatible with the other ingredients of the composition and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microbes may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Pharmaceutical compositions of the present invention may be administered to epithelial surfaces of the body orally, parenterally, topically, rectally, nasally, intravaginally, intracisternally. They are of course given by forms suitable for each administration route. For example, they are administered in tablets or capsule form, by injection, inhalation, eye lotion, ointment, etc., administration by injection, infusion or inhalation; topical by lotion or ointment; and rectal or vaginal suppositories.

The phrases “parenteral administration” and “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a sucrose octasulfate and/or an antibacterial, drug or other material other than directly into the central nervous system, such that it enters the subject's system and, thus, is subject to metabolism and other like processes, for example, subcutaneous administration.

In some methods, the compositions of the invention can be topically administered to any epithelial surface. An “epithelial surface” according to this invention is defined as an area of tissue that covers external surfaces of a body, or which lines hollow structures including, but not limited to, cutaneous and mucosal surfaces. Such epithelial surfaces include oral, pharyngeal, esophageal, pulmonary, ocular, aural, nasal, buccal, lingual, vaginal, cervical, genitourinary, alimentary, and anorectal surfaces.

Compositions can be formulated in a variety of conventional forms employed for topical administration. These include, for example, semi-solid and liquid dosage forms, such as liquid solutions or suspensions, suppositories, douches, enemas, gels, creams, emulsions, lotions, slurries, powders, sprays, lipsticks, foams, pastes, toothpastes, ointments, salves, balms, douches, drops, troches, chewing gums, lozenges, mouthwashes, rinses.

Conventionally used carriers for topical applications include pectin, gelatin and derivatives thereof, polylactic acid or polyglycolic acid polymers or copolymers thereof, cellulose derivatives such as methyl cellulose, carboxymethyl cellulose, or oxidized cellulose, guar gum, acacia gum, karaya gum, tragacanth gum, bentonite, agar, carbomer, bladderwrack, ceratonia, dextran and derivatives thereof, ghatti gum, hectorite, ispaghula husk, polyvinylpyrrolidone, silica and derivatives thereof, xanthan gum, kaolin, talc, starch and derivatives thereof, paraffin, water, vegetable and animal oils, polyethylene, polyethylene oxide, polyethylene glycol, polypropylene glycol, glycerol, ethanol, propanol, propylene glycol (glycols, alcohols), fixed oils, sodium, potassium, aluminum, magnesium or calcium salts (such as chloride, carbonate, bicarbonate, citrate, gluconate, lactate, acetate, gluceptate or tartrate).

Such compositions can be particularly useful, for example, for treatment or prevention of an unwanted cell, e.g., vaginal Neisseria gonorrhoeae, or infections of the oral cavity, including cold sores, infections of eye, the skin, or the lower intestinal tract. Standard composition strategies for topical agents can be applied to the antiinfective compounds or a pharmaceutically acceptable salt thereof in order to enhance the persistence and residence time of the drug, and to improve the prophylactic efficacy achieved.

For topical application to be used in the lower intestinal tract or vaginally, a rectal suppository, a suitable enema, a gel, an ointment, a solution, a suspension or an insert can be used. Topical transdermal patches may also be used. Transdermal patches have the added advantage of providing controlled delivery of the compositions of the invention to the body. Such dosage forms can be made by dissolving or dispersing the agent in the proper medium.

Compositions of the invention can be administered in the form of suppositories for rectal or vaginal administration. These can be prepared by mixing the agent with a suitable non-irritating carrier which is solid at room temperature but liquid at rectal temperature and therefore will melt in the rectum or vagina to release the drug. Such materials include cocoa butter, beeswax, polyethylene glycols, a suppository wax or a salicylate, and which is solid at room temperature, but liquid at body temperature and, therefore, will melt in the rectum or vaginal cavity and release the active agent.

Compositions which are suitable for vaginal administration also include pessaries, tampons, creams, gels, pastes, foams, films, or spray compositions containing such carriers as are known in the art to be appropriate. The carrier employed in the sucrose octasulfate/contraceptive agent should be compatible with vaginal administration and/or coating of contraceptive devices. Combinations can be in solid, semi-solid and liquid dosage forms, such as diaphragm, jelly, douches, foams, films, ointments, creams, balms, gels, salves, pastes, slurries, vaginal suppositories, sexual lubricants, and coatings for devices, such as condoms, contraceptive sponges, cervical caps and diaphragms.

For ophthalmic applications, the pharmaceutical compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions can be formulated in an ointment such as petrolatum. Exemplary ophthalmic compositions include eye ointments, powders, solutions and the like.

Powders and sprays can contain, in addition to sucrose octasulfate and/or antibiotic or contraceptive agent(s), carriers such as lactose, talc, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), proteins like serum albumin, sorbitan esters, oleic acid, lecithin, amino acids such as glycine, buffers, salts, sugars or sugar alcohols. Aerosols generally are prepared from isotonic solutions.

Compositions of the invention can also be orally administered in any orally-acceptable dosage form including, but not limited to, capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of sucrose octasulfate and/or antibiotic or contraceptive agent(s) as an active ingredient. A compound may also be administered as a bolus, electuary or paste. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Tablets, and other solid dosage forms, such as dragees, capsules, pills and granules, may be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the antiinfective agent(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Sterile injectable forms of the compositions of this invention can be aqueous or oleaginous suspension. These suspensions may be formulated according to techniques known in the art using suitable dispersing or wetting agents and suspending agents. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The sterile injectable preparation may also be a sterile injectable solution or suspension in a nontoxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The antiinfective agent or a pharmaceutically acceptable salt thereof will represent some percentage of the total dose in other dosage forms in a material forming a combination product, including liquid solutions or suspensions, suppositories, douches, enemas, gels, creams, emulsions, lotions slurries, soaps, shampoos, detergents, powders, sprays, lipsticks, foams, pastes, toothpastes, ointments, salves, balms, douches, drops, troches, lozenges, mouthwashes, rinses and others. Creams and gels for example, are typically limited by the physical chemical properties of the delivery medium to concentrations less than 20% (e.g., 200 mg/gm). For special uses, far less concentrated preparations can be prepared, (e.g., lower percent formulations for pediatric applications). For example, the pharmaceutical composition of the invention can comprise sucrose octasulfate in an amount of 0.001-99%, typically 0.01-75%, more typically 0.1-20%, especially 1-10% by weight of the total preparation. In particular, a preferred concentration thereof in the preparation is 0.5-50%, especially 0.5-25%, such as 1-10%. It can be suitably applied 1-10 times a day, depending on the type and severity of the condition to be treated or prevented.

Given the low toxicity of an antiinfective agent or a pharmaceutically acceptable salt thereof over many decades of clinical use as an anti-ulcerant (W. R. Garnett, Clin. Pharm. 1:307-314 (1982); R. N. Brogden et al., Drugs 27:194-209 (1984); D. M. McCarthy, New Eng J Med., 325:1017-1025 (1991)), an upper limit for the therapeutically effective dose is not a critical issue.

For prophylactic applications, the pharmaceutical composition of the invention can be applied prior to potential infection. The timing of application prior to potential infection can be optimized to maximize the prophylactic effectiveness of the compound. The timing of application will vary depending on the mode of administration, the epithelial surface to which it is applied, the surface area, doses, the stability and effectiveness of composition under the pH of the epithelial surface, the frequency of application, e.g., single application or multiple applications. One skilled in the art will be able to determine the most appropriate time interval required to maximize prophylactic effectiveness of the compound.

The practice of the present invention will employ, unless otherwise indicated, conventional techniques of cell biology, cell culture, molecular biology, genetics, microbiology, recombinant DNA, and immunology, which are within the skill of the art. Such techniques are explained fully in the literature. See, for example, Genetics; Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, NY (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Hames & S. J. Higgins eds. (1984)); the treatise, Methods In Enzymology (Academic Press, Inc., N.Y); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

### IX. The Role of Transcription Activation Factor Polypeptides in Biofilms

In one embodiment, the invention pertains to a method for dispersing or preventing the formation of a biofilm on a surface or in an area, by administering an effective amount of a transcription factor modulating compound, e.g., a HTH protein modulating compound, an AraC family polypeptide modulating compound, a MarA family polypeptide modulating compound, or a MarA inhibiting compound.

It has been discovered that the absence of MarA and its homologs has a negative effect on biofilm formation in E. coli. In order to confirm this finding genetically, plasmid encoded marA was transformed into an E. coli strain deleted of marA, soxS, and rob (triple knockout). The expression of MarA in this triple knockout restored biofliim formation in this host to a level that was comparable to that of the wild type host.

The term “biofilm” includes biological films that develop and persist at interfaces in aqueous and other environments. Biofilms are composed of microorganisms embedded in an organic gelatinous structure composed of one or more matrix polymers which are secreted by the resident microorganisms. The term “biofilm” also includes bacteria that are attached to a surface in sufficient numbers to be detected or communities of microorganisms attached to a surface (Costerton, J. W., et al. (1987) Ann. Rev. Microbiol. 41:435-464; Shapiro, J. A. (1988) Sci Am. 256:82-89; O'Toole, G. et al (2000) Annu Rev Microbiol. 54:49-79).

In another embodiment, the invention pertains to methods of treating biofilm associated states in a subject, by administering to said subject an effective amount of a transcription factor modulating compound, e.g., a MarA family inhibiting compound, such that the biofilm associated state is treated.

The term “biofilm associated states” includes disorders which are characterized by the presence or potential presence of a bacterial biofilm. Many medically important pathogens form biofilms and biofilm formation is often one component of the infectious process (Costerton, J. W. et al. (1999) Science 284:1318-1322). Examples of biofilm associated states include, but are not limited to, middle ear infections, cystic fibrosis, osteomyelitis, acne, dental cavities, and prostatitis. Biofilm associated states also include infection of the subject by one or more bacteria, e.g., Pseudomonas aeruginosa. One consequence of biofilm formation is that bacteria within biofilms are generally less susceptible to a range of different antibiotics relative to their planktonic counterparts.

Furthermore, the invention also pertains to methods for preventing the formation of biofilms on surfaces or in areas, by contacting the area with an effective amount of a transcription factor modulating compound, e.g., a MarA family inhibiting compound, etc.

Industrial facilities employ many methods of preventing biofouling of industrial water systems. Many microbial organisms are involved in biofilm formation in industrial waters. Growth of slime-producing bacteria in industrial water systems causes problems including decreased heat transfer, fouling and blockage of lines and valves, and corrosion or degradation of surfaces. Control of bacterial growth in the past has been accomplished with biocides. Many biocides and biocide formulations are known in the art. However, many of these contain components which may be environmentally deleterious or toxic, and are often resistant to breakdown.

The transcription factor inhibiting compounds, such as but not limited to AraC family inhibiting compounds and MarA family inhibiting compounds, of the present invention are useful in a variety of environments including industrial, clinical, the household, and personal care. The compositions of the invention may comprise one or more compounds of the invention as an active ingredient acting alone, additively, or synergistically against the target organism.

The MarA family inhibiting compounds and modulating compounds of the invention may be formulated in a composition suitable for use in environments including industry, pharmaceutics, household, and personal care. In an embodiment, the compounds of the invention are soluble in water. The modulating compounds may be applied or delivered with an acceptable carrier system. The composition may be applied or delivered with a suitable carrier system such that the active ingredient (e.g., transcription factor modulating compound of the invention such as a MarA family modulating compound, e.g., a MarA family polypeptide inhibiting compound) may be dispersed or dissolved in a stable manner so that the active ingredient, when it is administered directly or indirectly, is present in a form in which it is available in a advantageous way.

Also, the separate components of the compositions of the invention may be preblended or each component may be added separately to the same environment according to a predetermined dosage for the purpose of achieving the desired concentration level of the treatment components and so long as the components eventually come into intimate admixture with each other. Further, the present invention may be administered or delivered on a continuous or intermittent basis.

A transcription factor modulating compound, e.g., a MarA family modulating compound of the present invention, when present in a composition will generally be present in an amount from about 0.000001% to about 100%, more preferably from about 0.001% to about 50%, and most preferably from about 0.01% to about 25%.

For compositions of the present invention comprising a carrier, the composition comprises, for example, from about 1% to about 99%, preferably from about 50% to about 99%, and most preferably from about 75% to about 99% by weight of at least one carrier.

The transcription factor modulating compound, e.g., the MarA family polypeptide inhibiting compound, of the invention may be formulated with any suitable carrier and prepared for delivery in forms, such as, solutions, microemulsions, suspensions or aerosols. Generation of the aerosol or any other means of delivery of the present invention may be accomplished by any of the methods known in the art. For example, in the case of aerosol delivery, the compound is supplied in a finely divided form along with any suitable carrier with a propellant. Liquefied propellants are typically gases at ambient conditions and are condensed under pressure. The propellant may be any acceptable and known in the art including propane and butane, or other lower alkanes, such as those of up to 5 carbons. The composition is held within a container with an appropriate propellant and valve, and maintained at elevated pressure until released by action of the valve.

The compositions of the invention may be prepared in a conventional form suitable for, but not limited to topical or local application such as an ointment, paste, gel, spray and liquid, by including stabilizers, penetrants and the carrier or diluent with the compound according to a known technique in the art. These preparations may be prepared in a conventional form suitable for enteral, parenteral, topical or inhalational applications.

The present invention may be used in compositions suitable for household use. For example, compounds of the present invention are also useful as active antimicrobial ingredients in household products such as cleansers, detergents, disinfectants, dishwashing liquids, soaps and detergents. In an embodiment, the transcription factor modulating compound of the present invention may be delivered in an amount and form effective for the prevention, removal or termination of microbes.

The compositions of the invention for household use comprise, for example, at least one transcription factor modulating compound of the invention and at least one suitable carrier. For example, the composition may comprise from about 0.00001% to about 50%, preferably from about 0.0001% to about 25%, most preferably from about 0.0005% to about 10% by weight of the modulating compound based on the weight percentage of the total composition.

The transcription factor modulating compound of the present invention may also be used in hygiene compositions for personal care. For instance, compounds of the invention can be used as an active ingredient in personal care products such as facial cleansers, astringents, body wash, shampoos, conditioners, cosmetics and other hygiene products. The hygiene composition may comprise any carrier or vehicle known in the art to obtain the desired form (such as solid, liquid, semisolid or aerosol) as long as the effects of the compound of the present invention are not impaired. Methods of preparation of hygiene compositions are not described herein in detail, but are known in the art. For its discussion of such methods, The CTFA Cosmetic Ingredient Handbook, Second Edition, 1992, and pages 5-484 of A Formulary of Cosmetic Preparations (Vol. 2, Chapters 7-16) are incorporated herein by reference.

The hygiene composition for use in personal care comprise generally at least one modulating compound of the present application and at least one suitable carrier. For example, the composition may comprise from about 0.00001% to about 50%, preferably from about 0.0001% to about 25%, more preferably from about 0.0005% to about 10% by weight of the transcription factor modulating compound of the invention based on the weight percentage of the total composition.

The transcription factor modulating compound of the present invention may be used in industry. In the industrial setting, the presence of microbes can be problematic, as microbes are often responsible for industrial contamination and biofouling. Compositions of the invention for industrial applications may comprise an effective amount of the compound of the present invention in a composition for industrial use with at least one acceptable carrier or vehicle known in the art to be useful in the treatment of such systems. Such carriers or vehicles may include diluents, deflocculating agents, penetrants, spreading agents, surfactants, suspending agents, wetting agents, stabilizing agents, compatibility agents, sticking agents, waxes, oils, co-solvents, coupling agents, foams, antifoaming agents, natural or synthetic polymers, elastomers and synergists. Methods of preparation, delivery systems and carriers for such compositions are not described here in detail, but are known in the art. For its discussion of such methods, U.S. Pat. No. 5,939,086 is herein incorporated by reference. Furthermore, the preferred amount of the composition to be used may vary according to the active ingredient(s) and situation in which the composition is being applied.

The transcription factor modulating compounds, e.g., MarA family polypeptide inhibiting compounds, and compositions of the present invention may be useful in nonaqueous environments. Such nonaqueous environments may include, but are not limited to, terrestrial environments, dry surfaces or semi-dry surfaces in which the compound or composition is applied in a manner and amount suitable for the situation.

The transcription factor modulating compounds, e.g., MarA family polypeptide modulating compounds, e.g., MarA inhibiting compounds, of the present invention may be used to form contact-killing coatings or layers on a variety of substrates including personal care products (such as toothbrushes, contact lens cases and dental equipment), healthcare products, household products, food preparation surfaces and packaging, and laboratory and scientific equipment. Further, other substrates include medical devices such as catheters, urological devices, blood collection and transfer devices, tracheotomy devices, intraocular lenses, wound dressings, sutures, surgical staples, membranes, shunts, gloves, tissue patches, prosthetic devices (e.g., heart valves) and wound drainage tubes. Still further, other substrates include textile products such as carpets and fabrics, paints and joint cement. A further use is as an antimicrobial soil fumigant.

The transcription factor modulating compounds of the invention may also be incorporated into polymers, such as polysaccharides (cellulose, cellulose derivatives, starch, pectins, alginate, chitin, guar, carrageenan), glycol polymers, polyesters, polyurethanes, polyacrylates, polyacrylonitrile, polyamides (e.g., nylons), polyolefins, polystyrenes, vinyl polymers, polypropylene, silks or biopolymers. The modulating compounds may be conjugated to any polymeric material such as those with the following specified functionality: 1) carboxy acid, 2) amino group, 3) hydroxyl group and/or 4) haloalkyl group.

The composition for treatment of nonaqueous environments may be comprise at least one transcription factor modulating compound of the present application and at least one suitable carrier. In an embodiment, the composition comprises from about 0.001% to about 75%, advantageously from about 0.01% to about 50%, and preferably from about 0.1% to about 25% by weight of a transcription factor modulating compound of the invention based on the weight percentage of the total composition.

The transcription factor modulating compounds and compositions of the invention may also be useful in aqueous environments. “Aqueous environments” include any type of system containing water, including, but not limited to, natural bodies of water such as lakes or ponds; artificial, recreational bodies of water such as swimming pools and hot tubs; and drinking reservoirs such as wells. The compositions of the present invention may be useful in treating microbial growth in these aqueous environments and may be applied, for example, at or near the surface of water.

The compositions of the invention for treatment of aqueous environments may comprise at least one transcription factor modulating compound of the present invention and at least one suitable carrier. In an embodiment, the composition comprises from about 0.001% to about 50%, advantageously from about 0.003% to about 15%, preferably from about 0.01% to about 5% by weight of the compound of the invention based on the weight percentage of the total composition.

The present invention also provides a process for the production of an antibiofouling composition for industrial use. Such process comprises bringing at least one of any industrially acceptable carrier known in the art into intimate admixture with a transcription factor modulating compound of the present invention. The carrier may be any suitable carrier discussed above or known in the art.

The suitable antibiofouling compositions may be in any acceptable form for delivery of the composition to a site potentially having, or having at least one living microbe. The antibiofouling compositions may be delivered with at least one suitably selected carrier as hereinbefore discussed using standard formulations. The mode of delivery may be such as to have a binding inhibiting effective amount of the antibiofouling composition at a site potentially having, or having at least one living microbe. The antibiofouling compositions of the present invention are useful in treating microbial growth that contributes to biofouling, such as scum or slime formation, in these aqueous environments. Examples of industrial processes in which these compounds might be effective include cooling water systems, reverse osmosis membranes, pulp and paper systems, air washer systems and the food processing industry. The antibiofouling composition may be delivered in an amount and form effective for the prevention, removal or termination of microbes.

The antibiofouling composition of the present invention generally comprise at least one compound of the invention. The composition may comprise from about 0.001% to about 50%, more preferably from about 0.003% to about 15%, most preferably from about 0.01% to about 5% by weight of the compound of the invention based on the weight percentage of the total composition.

The amount of antibiofouling composition may be delivered in an amount of about 1 mg/l to about 1000 mg/l, advantageously from about 2 mg/l to about 500 mg/l, and preferably from about 20 mg/l to about 140 mg/l.

Antibiofouling compositions for water treatment generally comprise transcription factor modulating compounds of the invention in amounts from about 0.001% to about 50% by weight of the total composition. Other components in the antibiofouling compositions (used at 0.1% to 50%) may include, for example, 2-bromo-2-nitropropane-1,3-diol (BNPD), β-nitrostyrene (BNS), dodecylguanidine hydrochloride, 2,2-dibromo-3-nitrilipropionamide (DBNPA), glutaraldehyde, isothiazolin, methylene bis(thiocyanate), triazines, n-alkyl dimethylbenzylammonium chloride, trisodium phosphate-based, antimicrobials, tributyltin oxide, oxazolidines, tetrakis (hydroxymethyl)phosphonium sulfate (THPS), phenols, chromated copper arsenate, zinc or copper pyrithione, carbamates, sodium or calcium hypochlorite, sodium bromide, halohydantoins (Br, Cl), or mixtures thereof.

Other possible components in the compositions of the invention include biodispersants (about 0.1% to about 15% by weight of the total composition), water, glycols (about 20-30%) or Pluronic (at approximately 7% by weight of the total composition). The concentration of antibiofouling composition for continuous or semi-continuous use is about 5 to about 70 mg/l.

Antibiofouling compositions for industrial water treatment may comprise compounds of the invention in amounts from about 0.001% to about 50% based on the weight of the total composition. The amount of compound of the invention in antibiofouling compositions for aqueous water treatment may be adjusted depending on the particular environment. Shock dose ranges are generally about 20 to about 140 mg/l; the concentration for semi-continuous use is about 0.5× of these concentrations.

The invention also pertains, at least in part, to a method of regulating biofilm development. The method includes administering a composition which contains a transcription factor modulating compound of the invention. The composition can also include other components which enhance the ability of the composition to degrade biofilms.

The composition can be formulated as a cleaning product, e.g., a household or an industrial cleaner to remove, prevent, inhibit, or modulate biofilm development. Advantageously, the biofilm is adversely affected by the administration of the compound of the invention, e.g., biofilm development is diminished. These compositions may include compounds such as disinfectants, soaps, detergents, as well as other surfactants. Examples of surfactants include, for example, sodium dodecyl sulfate; quaternary ammonium compounds; alkyl pyridinium iodides; TWEEN 80, TWEEN 85, TRITON X-100; BRIJ 56; biological surfactants; rhamnolipid, surfactin, visconsin, and sulfonates. The composition of the invention may be applied in known areas and surfaces where disinfection is required, including but not limited to drains, shower curtains, grout, toilets and flooring. A particular application is on hospital surfaces and medical instruments. The disinfectant of the invention may be useful as a disinfectant for bacteria such as, but not limited to, Pseudomonadaceae, Azatobacteraceae, Rhizabiaceae, Mthylococcaceae, Halobacteriaceae, Acetobacteraceae, Legionellaceae, Neisseriaceae, and other genera.

The invention also pertains to a method for cleaning and disinfecting contact lenses. The method includes contacting the contact lenses with a solution of at least one compound of the invention in an acceptable carrier. The invention also pertains to the solution comprising the compound, packaged with directions for using the solution to clean contact lenses.

The invention also includes a method of treating medical indwelling devices. The method includes contacting at least one compound of the invention with a medical indwelling device, such as to prevent or substantially inhibit the formation of a biofilm. Examples of medical indwelling devices include catheters, orthopedic devices and implants.

A dentifrice or mouthwash containing the compounds of the invention may be formulated by adding the compounds of the invention to dentifrice and mouthwash formulations, e.g., as set forth in Remington's Pharmaceutical Sciences, 18th Ed., Mack Publishing Co., 1990, Chapter 109 (incorporated herein by reference in its entirety). The dentifrice may be formulated as a gel, paste, powder or slurry. The dentifrice may include binders, abrasives, flavoring agents, foaming agents and humectants. Mouthwash formulations are known in the art, and the compounds of the invention may be advantageously added to them.

In one embodiment, the invention pertains to each of the transcription factor modulating compounds described herein in Table 2, and in Formulae (I)-(VII).

The contents of all references, patent applications and patents, cited throughout this application are hereby expressly incorporated by reference. Each reference disclosed herein is incorporated by reference herein in its entirety. Any patent application to which this application claims priority is also incorporated by reference herein in its entirety.

The invention is further illustrated by the following examples, which should not be construed as further limiting.

### EXEMPLIFICATION OF THE INVENTION

### Example 1

### Synthesis of Selected Compounds of the Invention

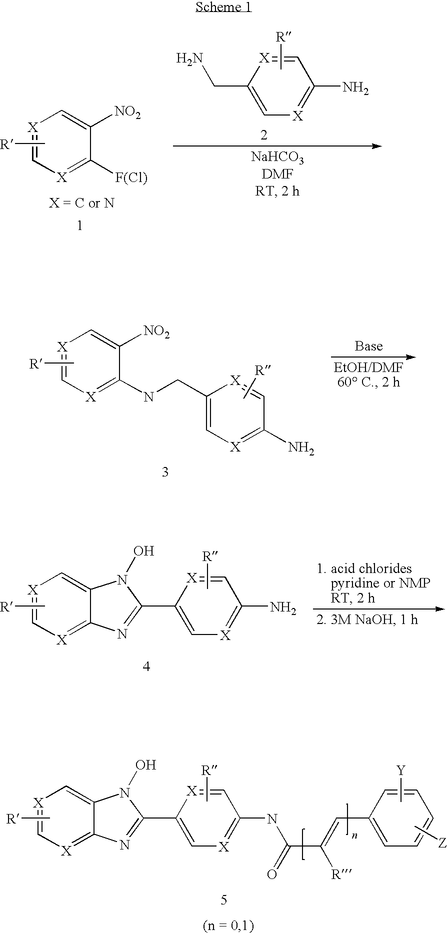

### Preparation of 4-aminobenzyl-(2,4-dinitro-phenyl)-amine Derivatives (3)

To a solution of 4-aminobenzyl amine derivatives (2) (225 mmol) and powdered NaHCO₃ (1125 mmol) in anhydrous DMF (300 mL) at was added 2,4-dinitrofluoro benzene (1) (150 mmol) dropwise at room temperature. After 2 hours, the solution was slowly diluted with water (1000 mL) to precipitate the product, which was collected on a fritted funnel rinsing with water until the eluent was colorless. The solid was further dried under high vacuum to afford a bright orange solid.

### Preparation of 6-nitro-2-(4-aminophenyl)-1-hydroxybenzimidazole Derivatives (4)

To a solution of N-(4-aminobenzyl)-2,4-dinitroaniline derivative (3) (74.9 mmol) in anhydrous EtOH (300 mL) and anhydrous DMF (75 mL) was slowly added sodium methoxide (30% w/w) (375 mmol) at room temperature under Argon atmosphere. After the addition, the solution was warmed to 60° C. for 2 hours. After cooling to ambient temperature, the solution was transferred to an Erlenmeyer flask or tall beaker through dilution with water (700 mL) and then acidified with saturated citric acid. The resulting precipitate was collected on a sintered funnel rinsing with water. The crude product was purified by recrystallization in hot EtOH to afford a brown solid.

### Preparation of N-acyl-6-nitro-2-(4-aminophenyl)-1-hydroxybenzimidazole Derivatives (5)

To a solution of 6-nitro-2-(4-aminophenyl)-1-hydroxybenzimidazole derivative (4) (1.00 mmol) in anhydrous pyridine (2.0 mL) was added acid chlorides (2.50 mmol) or the in situ formed mixed anhydrides at room temperature. After stirring for 2-3 hours, the solution was diluted with 3M NaOH (6.0 mL) and stirred for another hour. The deep amber solution was transferred to an Erlenmeyer flask or beaker through dilution with water (100 mL) and then acidified with saturated citric acid. The resulting precipitate was collected on a sintered funnel rinsing with water. The crude product was further purified either by preparatory HPLC, or by recrystallization in hot ethanol or a mixture of hot ethanol and chloroform.

### (E)-N-[4-(1-Hydroxy-6-nitro-1H-benzoimidazol-2-yl)-phenyl]-3-(4-[1,2,4]triazol-1-yl-phenyl)-acrylamide (Compound BI)

¹H NMR (300 MHz, DMSO-d₆): δ 10.60 (s, 1H), 9.38 (s, 1H), 8.36-8.32 (d, 3H), 8.28 (s, 1H), 8.15-8.11 (d, 1H), 7.99-7.93 (t, 4H), 7.87-7.82 (m, 3H), 7.73-7.68 (d, 1H), 6.96-6.91 (d, 1H). MS (M+1)=375.05

### (E)-N-[4-(1-Hydroxy-6-nitro-1H-benzoimidazol-2-yl)-phenyl]-3-(4-imidazol-1-yl-phenyl)-acrylamide (Compound BK)

¹H NMR (300 MHz, DMSO-d₆): δ 10.77 (s, 1H), 9.77 (s, 1H), 8.37-8.34 (m, 4H), 8.15-8.12 (dd, 1H), 7.98-7.92 (m, 7H), 7.85-7.82 (d, 1H), 7.76-7.71 (d, 1H), 7.08-7.03 (d, 1H). MS (M+1)=467.20

### 2-[4-(4-Fluoro-benzoylamino)-phenyl]-3-hydroxy-3H-benzoimidazole-5-carboxylic Acid Amide (Compound AD)

¹H NMR (300 MHz, DMSO-d₆); δ 10.55 (s, 1H), 8.30 (d, 2H), 8.18-7.96 (m, 6H), 7.87 (d, 1H), 7.70 (d, 1H), 7.39 (t, 3H), 6.78 (d, 2H), 3.02 (s, 6H). MS (M+1)=391.20

### (E)-N-[2-Fluoro-4-(1-hydroxy-6-nitro-1H-benzoimidazol-2-yl)-phenyl]-3-(4-fluoro-phenyl)-acrylamide: (Compound AZ)

¹H NMR (300 MHz, DMSO-d₆); δ 10.23 (s, 1H), 8.49-8.39 (t, 1H), 8.38 (s, 1H), 8.22-8.12 (m, 3H), 7.87-7.84 (d, 1H), 7.72-7.63 (m, 3H), 7.33-7.28 (t, 2H), 7.14-7.09 (d, 1H). MS (M+1)=375.05

### 4-Acetyl-N-[2-fluoro-4-(1-hydroxy-6-nitro-1H-benzoimidazol-2-yl)-phenyl]-benzamide (Compound BA)

¹H NMR (300 MHz, DMSO-d₆): δ 12.81 (br s, 1H), 10.57 (s, 1H), 8.42 (s, 1H), 8.41-8.13 (m, 7H), 7.98 (t, 1H), 7.89-7.86 (d, 1H), 2.66 (s, 3H). MS (M+1)=435.10

### (E)-3-(4-Acetyl-phenyl)-N-[4-(1-hydroxy-6-nitro-1H-benzoimidazol-2-yl)-phenyl]-acrylamide (Compound BQ)

¹H NMR (300 MHz, DMSO-d₆): δ 12.65 (s, 1H), 10.65 (s, 1H), 8.33-8.36 (m, 3H), 8.13 (dd, 1H), 8.03 (d, 2H), 7.94 (d, 2H), 7.78-7.84 (m, 3H), 7.7 (d, 1H), 7.0 (d, 1H), 2.6 (s, 3H). MS (M−1)=441

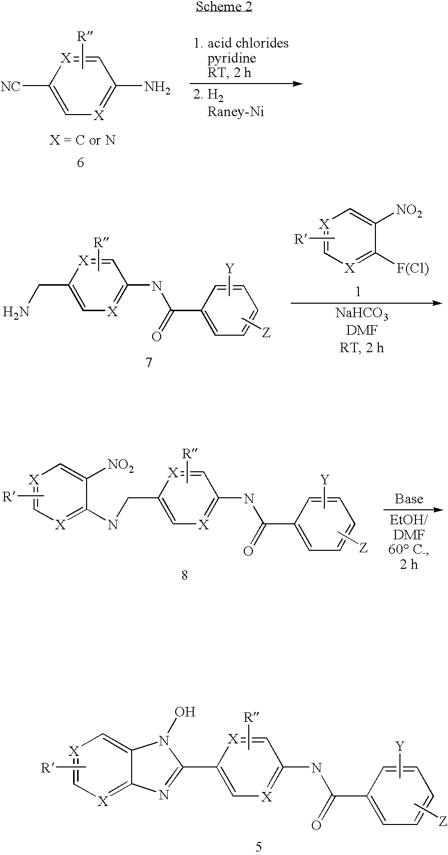

### Preparation of 4-phenylamidobenzylamine Derivatives (7)

To a solution of 4-cyanoaniline derivative (6) (225 mmol) in N-methylpyrrolidone (180 mL), was added an acid chloride (225.4 mmol) over a period of 3-5 minutes with vigorous stirring. After stirring the reaction mixture for about 5 hours (until the HPLC monitoring of the reaction indicated a complete consumption of the starting materials), it was poured into about 1400 mL of water at room temperature and the resulting suspension was stirred for about 1 hour. The precipitate was filtered, washed with 4×500 mL portions of water, and dried. A second crop of solid can be obtained from the filtrate and washings. In a pressure reactor, 4-phenylamido benzonitrile intermediate (98 mmol) was dissolved in anhydrous THF (940 mL), and the solution was purged with argon for 2-3 minutes, followed by the addition of 11 mL of the uniformly suspended catalyst (Raneye nickel 2400, suspension in water). After addition of a small amount of methanol to the suspension, the reactor was pressurized at 55 psi of H₂ while stirring vigorously. LC-MS monitoring of the reaction indicated a complete conversion of the starting material to the corresponding amine within 2.5 hours. The reaction mixture was filtered over a bed of diatomaceous earth (e.g., Celite®), and washed with 3×100 mL portions of anhydrous THF. The combined filtrates were evaporated to dryness, and further dried under high vacuum to afford white colored solid.

### Preparation of N-{4-[(2,4-dinitrophenylamino)-methyl]-phenyl}-benzamide Derivatives (8)

To a solution of 4-phenylamidobenzylamine derivatives (7) (225 mmol) and powdered NaHCO₃ (1125 mmol) in anhydrous DMF (300 mL) at was added 2,4-dinitrofluoro benzene (1) (150 mmol) dropwise at room temperature. After 2 hours, the solution was slowly diluted with water (1000 mL) to precipitate the product, which was collected on a fritted funnel rinsing with water until the eluent was colorless. The solid was further dried under high vacuum to afford the product as a bright orange solid.

### Preparation of N-[4-(1-hydroxy-6-nitro-1H-benzoimidazol-2-yl)-phenyl]-benzamide Derivatives (5)

To a solution of N-{4-[(2,4-dinitrophenylamino)-methyl]-phenyl}-benzamide derivatives (8) (74.9 mmol) in anhydrous EtOH (300 mL) and anhydrous DMF (75 mL) was slowly added sodium methoxide (30% w/w) (69.1 g, 375 mmol) at room temperature under Argon atmosphere. After the addition, the solution was warmed to 60° C. for 2 hours. After cooling to ambient temperature, the solution was transferred to an Erlenmeyer flask or tall beaker through dilution with water (700 mL) and then acidified with saturated citric acid. The resulting precipitate was collected on a sintered funnel rinsing with water. The crude product was purified by recrystallization in hot EtOH.

### [5-(4-Fluoro-benzoylamino)-2-(1-hydroxy-6-nitro-1H-benzoimidazol-2-yl)-phenoxymethyl]-phosphonic Acid: (Compound AR)

¹H NMR (300 MHz, DMSO-d₆): δ 10.57 (s, 1H), 8.30 (s, 1H), 8.29-8.06 (m, 3H), 7.86-7.83 (d, 2H), 7.67-7.44 (t, 2H), 7.41-7.38 (t, 2H), 4.36-4.32 (d, 2H). MS (M−1)=501

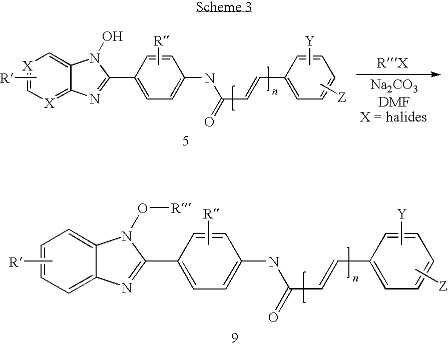

### Preparation of N-[4-(1-alkyloxy-6-nitro-1H-benzoimidazol-2-yl)-phenyl]-benzamide Derivatives (9)

A suspension of N-[4-(1-hydroxy-6-nitro-1H-benzoimidazol-2-yl)-phenyl]-benzamide derivatives (5) (0.19 mmol) and anhydrous sodium carbonate (0.96 mmol) in 3 mL of DMF, was treated with substituted alkyl halide derivatives (0.25 mmol) and stirred at RT. After 24 h, the reaction mixture was poured into 20 mL of water and stirred for 2 h. The precipitate formed was filtered, washed with 4×10 mL portions of water and dried under vacuum to afford the product.

### (2-{2-[4-(4-Fluoro-benzoylamino)-phenyl]-6-nitro-benzoimidazol-1-yloxy}-ethyl)-trimethyl-ammonium (Compound W)

¹H NMR (300 MHz, DMSO-d₆): δ 10.62 (s, 1H), 8.72 (s, 1H), 8.22 (t, 3H), 8.15-8.12 (m, 4H), 7.93 (d, 1H), 7.41 (t, 2H), 4.78 (t, 2H), 3.99 (t, 2H), 3.21 (s, 9H). MS (m/z, M)=478.39

### {2-[4-(4-Fluoro-benzoylamino)-phenyl]-6-nitro-benzoimidazol-1-yloxy}-acetic Acid (Compound V)

¹H NMR (300 MHz, DMSO-d₆): δ 10.55 (s, 1H), 8.78 (d, 1H), 8.30 (d, 2H), 8.20-8.00 (m, 5H), 7.85 (d, 1H), 7.41 (t, 2H), 5.02 (s, 2H). MS (M+1)=451.20

### (2-{4-[(E)-3-(4-Fluoro-phenyl)-acryloylamino]-phenyl}-6-nitro-benzoimidazol-1-yloxymethyl)-phosphonic Acid Diethyl Ester (Compound AS)

¹H NMR (300 MHz, DMSO-d₆): δ 10.62 (s, 1H), 8.60 (d, 1H), 8.30 (d, 2H), 8.23 (dd, 1H), 7.99 (d, 2H), 7.91 (d, 1H), 7.79-7.67 (m, 3H), 7.34 (dd, 2H), 6.86 (d, 1H), 4.95 (d, 2H), 4.19 (q, 4H), 1.33 (t, 6H). MS (M−1)=567

### (2-{4-[(E)-3-(4-Fluoro-phenyl)-acryloylamino]-phenyl}-6-nitro-benzoimidazol-1-yloxymethyl)-phosphonic Acid (Compound AL)

¹H NMR (300 MHz, DMSO-d₆): δ 10.55 (s, 1H), 8.56 (d, 1H), 8.32 (d, 2H), 8.17 (dd, 1H), 7.92 (d, 2H), 7.86 (d, 1H), 7.74-7.61 (m, 3H), 7.30 (dd, 2H), 6.80 (d, 1H), 4.50 (d, 2H). MS (M−1)=511

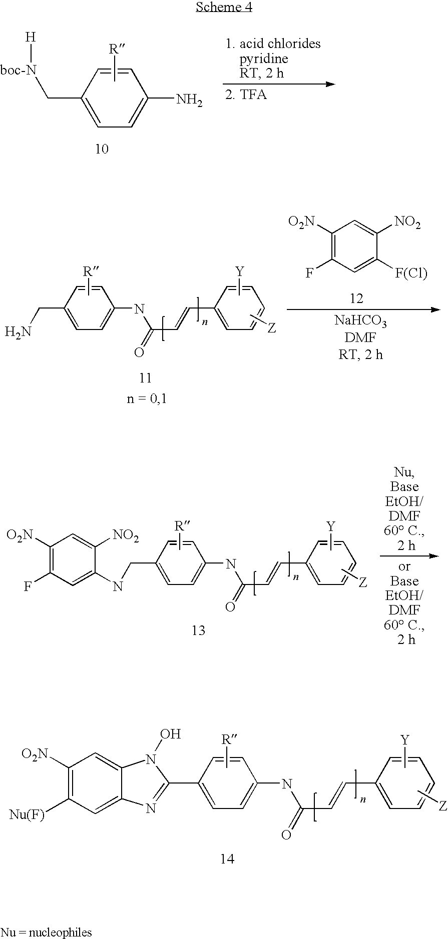

### Preparation of (E)-N-(4-Aminomethyl-phenyl)-3-phenyl-acrylamide Derivatives (11)

To a solution of 4-(tert-butoxycarbonyl-aminomethyl)-aniline derivative (0.94 mmol) in 7 mL of NMP, acid chloride derivative (1.0 mmol) was added and the reaction was stirred at room temperature for 40 minutes. It was then poured in 100 mL of water while stirring. The precipitate was filtered, washed with water (5×15 mL) and dried to give the boc-protected product. A solution of the boc-protected product (0.83 mmol) in 10 mL of TFA was stirred at room temperature for 20 minutes. It was then diluted with 200 mL of diethyl ether and the suspension stirred for another 10 minutes. The precipitate was filtered, washed with 3×20 mL of diethyl ether, and dried under vacuum for 6 hours to give the product (11) as its TFA salt.

### Preparation of (E)-N-{4-[(5-Fluoro-2,4-dinitro-phenylamino)-methyl]-phenyl}-3-phenyl-acrylamide (13)

To a solution of 1,5-difluoro-2,4-dinitrobenzene (12) (2.0 mmol) in 8 mL of DMF, was added sodium bicarbonate (10.0 mmol) and the compound (11) (2.0 mmol) and the reaction mixture was refluxed for 10 hours. The reaction was poured in ice-water to give a precipitate. The precipitate was filtered, washed with water, and dried under vacuum to give the desired product.

### Preparation of (E)-N-[4-(5-ethyloxy-1-hydroxy-6-nitro-1H-benzoimidazol-2-yl)-phenyl]-3-phenyl-acrylamide (14)

To a solution of (E)-N-{4-[(5-Fluoro-2,4-dinitro-phenylamino)-methyl]-phenyl}-3-phenyl-acrylamide (13) (0.44 mmol) in ethanol (10 mL) and DMF (10 mL), was added sodium hydride (2.2 mmol). The reaction mixture was heated at 60° C. for 3 hours. After cooling to room temperature, it was poured into ice-water, and acidified with aqueous citric acid. The resulting precipitation was collected, washed with water, and dried in vacuo to yield the product as a yellow solid.

### (E)-3-(4-Fluoro-phenyl)-N-[4-(1-hydroxy-5-methyl-6-nitro-1H-benzoimidazol-2-yl)-phenyl]-acrylamide (Compound BC)

¹H NMR (300 MHz, DMSO-d₆): δ 12.57 (s, 1H), 10.52 (s, 1H), 8.31 (d, 2H), 8.16 (s, 1H), 7.91 (d, 2H), 7.74-7.62 (m, 4H), 7.30 (dd, 2H), 6.82 (d, 1H), 2.63 (s, 3H). MS (M+1)=433

### (E)-N-[4-(5-Ethoxy-1-hydroxy-6-nitro-1H-benzoimidazol-2-yl)-phenyl]-3-(4-fluoro-phenyl)-acrylamide (Compound BJ)

¹H NMR (300 MHz, DMSO-d₆): δ 12.41 (s, 1H), 10.51 (s, 1H), 8.28 (d, 2H), 8.05 (s, 1H), 7.91 (d, 2H), 7.72 (dd, 2H), 7.64 (d, 1H), 7.49 (s, 1H), 7.30 (dd, 2H), 6.82 (d, 1H), 4.22 (q, 2H), 1.36 (t, 3H). MS (M+1)=463

### N-[4-(1-Hydroxy-5-methyl-6-nitro-1H-benzoimidazol-2-yl)-phenyl]-4-oxazol-5-yl-benzamide (Compound BL)

¹H NMR (300 MHz, DMSO-d₆): δ 12.47 (s, 1H), 10.67 (s, 1H), 8.50 (s, 1H), 8.32 (d, 2H), 8.18 (s, 1H), 8.10 (d, 2H), 8.01 (d, 2H), 7.88 (d, 2H), 7.84 (s, 1H), 7.69 (s, 1H), 2.62 (s, 3H). MS (M+1)=456

### N-[4-(5-Dimethylamino-1-hydroxy-6-nitro-1H-benzoimidazol-2-yl)-phenyl]-3-(4-fluorocinnamyl)-amide: (Compound BN)

¹H NMR (300 MHz, DMSO-d₆): δ 12.32 (s, 1H), 10.51 (s, 1H), 8.27 (d, 2H), 7.97 (s, 1H), 7.90 (d, 2H), 7.71 (dd, 2H), 7.65 (d, 1H), 7.49 (s, 1H), 7.30 (dd, 2H), 6.82 (d, 1H), 2.75 (s, 6H). MS (M+1)=462

### N-[4-(5-Fluoro-1-hydroxy-6-nitro-1H-benzoimidazol-2-yl)-phenyl]-3-(4-fluoro-cinnamyl)-amide (Compound BO)

¹H NMR (300 MHz, DMSO-d₆): δ 12.72 (s, 1H), 10.55 (s, 1H), 8.33 (d, 2H), 8.28 (d, 1H), 7.92 (d, 2H), 7.81 (d, 1H), 7.75-7.70 (m, 2H), 7.64 (d, 1H), 7.30 (dd, 2H), 6.82 (d, 1H). MS (M+1)=437

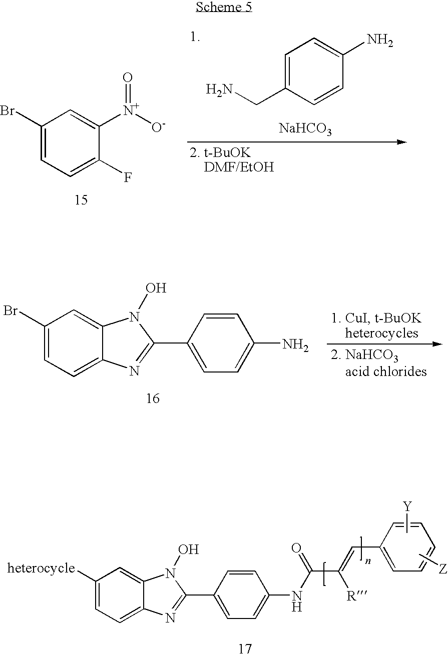

### Preparation of 6-bromo-2-(4-aminophenyl)-1-hydroxybenzimidazole (16)

To a solution of 4-aminobenzyl amine (35.4 mL, 313 mmol) and powdered NaHCO₃(158 g, 1875 mmol) in anhydrous DMF (500 mL) at room temperature was added a solution of 4-bromo-1-fluoro-2-nitrobenzene (15) (31.4 mL, 250 mmol) in anhydrous DMF (50 mL) dropwise via addition funnel over a 1 hour period. After another 4 hours or as determined complete by HPLC, the solution was diluted with anhydrous absolute ethanol (1000 mL) and powdered potassium tert-butoxide (140 g, 1250 mmol) was added in portions. This solution was subsequently heated to 60° C. for 6 hours. After cooling to room temperature, the solution was poured into stirring solution of water (4 L), then adjusted to pH 6 with 1M HCl. The slowly stirring suspension was cooled with an ice bath to facilitate solidification. The suspended product was collected on a fine fritted funnel rinsing with water until the eluent was colorless. The orange solid was further dried under high vacuum.

### Preparation of 6-pyrazole-2-(4-aminophenyl)-1-hydroxybenzimidazole

A 20 mL Biotage microwave vial was charged with 6-bromo-2-(4 aminophenyl)-1-hydroxybenzimidazole (16) (1.52 g, 5.00 mmol), N,N′-dimethylethylenediamine (1.10 mL, 10.0 mmol), CuI (0.952 g, 5.00 mmol), pyrazole (1.36 g, 20.0 mmol) and potassium tert-butoxide (2.24 g, 20.0 mmol) and anhydrous DMSO (20 mL). The secured vial was placed into a Biotage microwave reactor with a temperature setting of 195° C. for 45 minutes. After cooling, the vial was opened and poured into a rapidly stirring water solution. The resulting suspension was filtered through a plug of Celite rinsing with 0.5M NaOH. The water solution was loaded onto a prepared DVB column. After loading, the product was eluted with CH₃CN. The CH₃CN was removed under reduced pressure. The resulting water solution was cooled to 0° C. by an ice bath then adjusted to pH 6 with 1M HCl to precipitate the product 17. The resulting solid was collected onto a fine fritted funnel rinsing with cold water to afford a light brown solid to afford 1.52 g in 70% yield. The product was further dried under high vacuum.

### Preparation (E)-3-(4-Fluoro-phenyl)-N-[4-(1-hydroxy-6-pyrazol-1-yl-1H-benzoimidazol-2-yl)-phenyl]-acrylamide (Compound CL)

To a solution of 6-pyrazole-2-(4-aminophenyl)-1-hydroxybenzimidazole (0.78 g, 2.50 mmol) and NaHCO₃ (0.84 g, 10.0 mmol) in anhydrous CH₃CN (20 mL) and DMPU (5 mL) at room temperature was added 4-fluorocinnamoyl chloride (1.15 g, 6.25 mmol). After 6 hours, the solution was diluted with 3M NaOH (25 mL) and stirred for another 2 hours. The solution was transferred to another flask through dilution with water (100 mL) and then acidified with saturated citric acid. The resulting precipitate was collected on a sintered funnel rinsing with water. The crude product was further purified by recrystallization in hot ethanol or a mixture of hot ethanol and chloroform. ¹H NMR (DMSO-d6) δ 10.49 (s, 1H), 8.61 (s, 1H), 8.33 (m, 2H), 7.94-7.63 (m, 9H), 7.32 (m, 2H), 6.84 (m, 1H), 6.55 (s, 1H). LC/MS (m+1) 440.

### Preparation of 4-Acetyl-N-[4-(1-hydroxy-6-pyrazol-1-yl-1H-benzoimidazol-2-yl)-phenyl]-benzamide (Compound CM)

To a solution of 6-pyrazole-2-(4-aminophenyl)-1-hydroxybenzimidazole (0.78 g, 2.50 mmol) and NaHCO₃ (0.84 g, 10.0 mmol) in anhydrous CH₃CN (20 mL) and DMPU (5 mL) at room temperature was added 4-acetylbenzyl chloride (1.14 g, 6.25 mmol). After 6 hours, the solution was diluted with 3M NaOH (25 mL) and stirred for another 2 hours. The solution was transferred to another flask through dilution with water (100 mL) and then acidified with saturated citric acid. The resulting precipitate was collected on a sintered funnel rinsing with water. The crude product was further purified by recrystallization in hot ethanol or a mixture of hot ethanol and chloroform. ¹H NMR (DMSO-d6) δ 10.61 (s, 1H), 8.69-7.77 (m, 13H), 6.60 (1, 1H), 2.63 (s, 3H). LC/MS (m+1) 438.

### Preparation of 4-Acetyl-N-[4-(1-hydroxy-6-imidazol-1-yl-1H-benzoimidazol-2-yl)-phenyl]-benzamide: (Compound CN)

To a solution of 6-imidazole-2-(4-aminophenyl)-1-hydroxybenzimidazole (0.78 g, 2.50 mmol) and NaHCO₃ (0.84 g, 10.0 mmol) in anhydrous CH₃CN (20 mL) and DMPU (5 mL) at room temperature was added 4-acetylbenzyl chloride (1.14 g, 6.25 mmol). After 6 hours, the solution was diluted with 3M NaOH (25 mL) and stirred for another 2 hours. The solution was transferred to another flask through dilution with water (100 mL) and then acidified with saturated citric acid. The resulting precipitate was collected on a sintered funnel rinsing with water. The crude product was further purified by recrystallization in hot ethanol or a mixture of hot ethanol and chloroform.

¹H NMR (DMSO-d6) δ 10.63 (s, 1H), 8.32-7.46 (m, 13H), 7.13 (1, 1H), 2.68 (s, 3H). LC/MS (m+1) 438.

### Example 2

### SoxS Gel Shift Assay of Compounds

The compounds are diluted in DMSO to the required concentration and added to the appropriate wells. Protein (SoxS) was added to the wells in EMSA buffer at a concentration that was determined to cause a 50% shift of the DNA. The plates are then covered, mixed and shaked for 30 minutes at room temperature to allow for compound-protein binding.

Ten μl of DNA mix (2.4 μl 5×EMSA buffer, 0.2 μl poly(dIdC), 1 μl ³³P-DNA probe, 7.4 μl dH₂O per reaction) are then added to each well. The final DNA concentrations are approximately 1 nM. The samples are then mixed for 15 minutes at room temperature which allows protein-DNA complexes to form.

Electrophoresis is started at approximately 110V and the gels are pre-run for 10-15 minutes. Five μl of gel loading buffer is then added to each sample and mixed. Fifteen μl of each sample are then loaded onto gel. The gel is electrophoresed at 110V for approximately 2 hours or until the bromophenol blue marker approached the bottom of the gel. The gel is then transferred to Whatman filter paper, covered, and dried at 80° C. for approximately 30 minutes. Autoradiography film is exposed to the gel overnight and developed.

### Example 3

### Development of Luminescence Assays

A quantitative chemiluminescence-based assay is being used to measure the DNA binding activity of various MarA (AraC) family members. With this technique, biotinylated double-stranded DNA molecule (2 nM) is incubated with a MarA (AraC) protein (20 nM) fused to 6-histidine (6-His) residues in a streptavidin coated 96-well microtiter (white) plate (Pierce Biotechnology, Rockford, Ill.). Unbound DNA and protein is removed by washing and a primary monoclonal anti-6His antibody is subsequently added. A second washing is performed and a secondary HRP-conjugated antibody is then added to the mixture. Excess antibody is removed by a third wash step and a chemiluminescence substrate (Cell Signaling Technology, Beverly, Mass.) is added to the plate. Luminescence is read immediately using a Victor V plate reader (PerkinElmer Life Sciences, Wellesley, Mass.). Compounds that inhibit the binding of the protein to the DNA result in a loss of protein from the plate at the first wash step and are identified by a reduced luminescence signal. The concentration of compound necessary to reduce signal by 50% (EC₅₀/IC₅₀) can be calculated using serial dilutions of the inhibitory compounds. Also, single transcription factor modulators that affect different transcription factors have been identified.

### Example 4

### In Vivo Activity of Mar Inhibitors in Pyelonephritis Model of Infection

Groups of female CD1 mice (n=6) are diuresed and infected with E. coli UPEC strain C189 via intravesicular inoculation. Subsequently, mice are dosed with a transcription factor modulator (25 mg/kg), a control compound, e.g., SXT (Qualitest Pharmaceuticals, Huntsville, Ala.), or vehicle alone (0 mg/kg), via an oral route of administration at the time of infection and once a day for 4 days thereafter, to maintain a constant level of drug in the mice. After a 5-day period of infection and prior to sacrifice via CO₂/O₂ asphyxiation, a urine sample is taken by gentle compression of the abdomen. Following asphyxiation, the bladder and kidneys are removed aseptically. Urine volumes and individual organ weights are recorded, the organs are suspended in sterile PBS containing 0.025% Triton X-100, and then homogenized. Serial 10-fold dilutions of the urine samples and homogenates are plated onto McConkey agar plates to determine CFU/ml of urine or CFU/gram of organ.

Efficacy in these experiments were defined as a ≧2-log decrease in CFU/ml of urine or CFU/g organ.

### Example 5

### In Vitro Activity of Mar Inhibitors Against Lcrf (Virf) from Y. pseudotuberculosis

The MarA (AraC) family member LcrF (VirF) was cloned, expressed and purified from Y. pseudotuberculosis. The purified protein was used to develop a cell-free system to monitor DNA-protein interactions in vitro. The activities of Mar inhibitors were surveyed against LcrF to identify inhibitory activity and % cytotoxicity in whole cell assays at 50 μg/mL. The EC₅₀'s for some of the compounds of the invention are summarized in Table 3 below. Compounds with excellent inhibition are indicated with “***” (less than 10 EM), compounds with very good inhibition with “**” (between 10.1 and 25 μM) and good inhibition with “*” (greater than 25.1 μM). Compounds that were not tested are represented by “NT,” and compounds that were not active are represented by “—.”

### Example 6

### Activity of Mar Inhibitors in Whole Cell Systems

Type III secretion, the process whereby cytotoxic proteins (Yops) are secreted from a bacterium into a host cell, in pathogenic Yersinia spp. is regulated by LcrF. Wild type Y. pseudotuberculosis are toxic toward J774 tissue culture cells whereas bacteria bearing a mutation in either yopJ (a Yop that inhibits eukaryotic signaling pathways) or lcrF. The cytotoxicity of wild type Y. pseudotuberculosis was exploited in order to screen compounds for their ability to penetrate the intact bacterial cell and prevent type III secretion by binding to an inactivating LcrF function.

The CytoTox 96® assay kit from Promega was used for this assay. Briefly, J774 macrophages were plated out at 2×10⁴ cells per well in 96-well plates on the day prior to infection. Yersinia pseudotuberculosis were grown overnight at 26° C. in 2×YT media and then diluted 1:25 or 1:40 the following morning into 2×YT supplemented with 20 mM MgCl₂ and 20 mM sodium oxalate. The cultures were grown for a further 90 min at 26° C. and then shifted to 37° C. for 90 minutes. The temperature shift and the sodium oxalate, which chelates calcium, lead to induction of LcrF expression. Later experiments also included the YPIIIpB1 ΔJ (YopJ mutant) and YPIIIpIB1 ΔLcrF (LcrF mutant). YPIIIpIB1 ΔJ is a YopJ deletion mutant and any cytotoxicity that is unrelated to YopJ (i.e. lps-mediated) will be seen with this strain. The OD600 was measured and the culture adjusted to an OD600 of 1.0. This should correspond to approximately 1.25×10⁹ cells/mL. Dilutions were prepared in DMEM (the J774 culture media) at different multiplicity of infections (MOIs), assuming J774 cell density of 2×10⁴. Yersinia pseudotuberculosis were added in 10 μl aliquots and cells were incubated at 37° C. either in a chamber with a CO₂ generating system, or later, in a tissue culture incubator with 5% CO₂ for 2 hours. Gentamicin was then added to a final concentration of 50 μg/ml and the incubations were continued either for a further 2-3 h or overnight. Controls were included for media alone, target cell spontaneous lysis, target cell maximum lysis and effector cell spontaneous lysis. For maximum lysis, triton X-100 was added to a final concentration of 0.8% 45 minutes prior to termination of the experiment. Supernatants containing released LDH were harvested following centrifugation at 1,000 rpm for 5 minutes. Supernatants were either frozen overnight or assayed immediately. 50 μl of supernatant was mixed with 50 μl fresh assay buffer and incubated in the dark for 30 minutes 50 μl of stop solution was added to each well and the plates were read at 490 nm. In Table 3 below, the percent cytotoxicity of a bacteria treated with a compound of the invention compared to untreated bacteria is given. Compounds that exhibited a percent cytotoxicity above 75% at 50 μg/mL are indicated with “*.” Compounds with cytotoxicities below 75% at 50 μg/mL are indicated with “**.”

### Example 7

### In Vitro Activity of Mar Inhibitors Against ExsA from Pseudomonas aeruginosa

The MarA (AraC) family member ExsA was cloned, expressed and purified from P. aeruginosa. The purified protein was used to develop a cell-free system to monitor DNA-protein interactions in vitro. Individual Mar inhibitors were tested against ExsA in dose response studies to generate an EC₅₀ for each compound, the concentration required to inhibit 50% of ExsA DNA binding in vitro. The EC₅₀'s for some of the compounds of the invention are summarized in Table 3 below. Compounds with excellent inhibition are indicated with “***” (less than 10 μM), compounds with very good inhibition with “**” (between 10.1 and 25 μM) and good inhibition with “*” (greater than 25.1 μM). Compounds that were not tested are represented by “NT,” and compounds that were not active are represented by “—.”

### Example 8

### Activity of Mar Inhibitors in Whole Cell Systems

In pathogenic P. aeruginosa, type III secretions are regulated by ExsA. Type III secretion is the process in which cytotoxic proteins (ExoU, ExoT, etc.) are secreted from a bacterium into a host cell. Wild type P. aeruginosa are toxic toward J774 tissue culture cells whereas bacteria bearing a mutation in exsA are not. In this example, the cytotoxicity of wild type P. aeruginosa was exploited to screen compounds for their ability to penetrate the intact bacterial cell and prevent type III secretion by binding to an inactivating ExsA function.

The CytoTox 96® assay kit from Promega was used for this assay. Briefly, J774 macrophage-like cells were plated out at 5×10⁴ cells per well in 96-well plates on the day prior to infection. P. aeruginosa were grown overnight at 37° C. in Luria Broth and then diluted 1:25 in MinS, a minimal salt media containing the calcium chelator trisodium nitriloacetate. Experiments also included the WT□ExsA mutants, in which the entire exsA coding sequence has been deleted. Mar inhibitors were added to the MinS cultures at a concentration of 50 μg/mL and the cultures were grown for a further 3 hours at 37° C. The shift to a calcium free media leads to induction of ExsA expression. Cultures were grown to an OD600 of 1.0, approximately 1×10⁹ cells/mL. Dilutions were prepared in DMEM (the J774 culture media) at different multiplicity of infections (MOIs), assuming J774 cell density of 5×10⁴. Media in the J774 cell wells was replaced with DMEM containing 50 μg/mL of Mar inhibitors. P. aeruginosa were added to J774 cells in 10 μl aliquots, plates were centrifuged at 1,000 rpm for 5 minutes to synchronize infection and then incubated in a tissue culture incubator with 5% CO₂ for 2 h. Controls were included for media alone, target cell spontaneous lysis, target cell maximum lysis, and Mar inhibitors with J774 cells alone. For target cell maximum lysis, 10 P of the CytoTox 96® assay kit lysis solution was added to untreated J774 cells 30 minutes prior to termination of the experiment. Supernatants containing released LDH were harvested following centrifugation at 1,000 rpm for 5 minutes. Supernatants were stored frozen overnight or assayed immediately. 50 μl of supernatant was mixed with 50 μl fresh LDH substrate solution and incubated in the dark for 30 minutes. 50 μl of stop solution was added to each well and the plates were read at 490 nm. In Table 3 below, compounds with percent cytotoxicities above 75% at 50 mg/mL are indicated with “*.” Compounds with cytotoxicities below 75% at 50 mg/mL are indicated with “**.”

TABLE 3

| | | | Cytotoxicity at | Cytotoxicity at |
|---|---|---|---|---|
| | LcrF EC₅₀ | ExsA EC₅₀ | 50 μg/mL | 50 μg/mL |
| Code | (μM) | (μM) | Yersinia | Pseudomonas |
| A | ** | ** | * | * |
| B | *** | *** | ** | ** |
| C | * | *** | ** | ** |
| D | *** | *** | ** | * |
| E | ** | *** | ** | * |
| F | *** | ** | * | ** |
| G | ** | *** | * | ** |
| H | * | * | * | NT |
| I | * | — | * | NT |
| J | * | *** | ** | * |
| K | * | ** | * | * |
| L | — | NT | * | NT |
| M | * | * | * | NT |
| N | * | NT | * | * |
| P | ** | ** | * | * |
| R | * | ** | ** | * |
| S | ** | ** | * | * |
| T | *** | *** | ** | * |
| U | * | * | * | NT |
| V | * | NT | * | NT |
| X | ** | ** | * | * |
| Y | ** | *** | * | * |
| Z | ** | * | * | NT |
| AA | ** | ** | * | * |
| AB | ** | ** | * | * |
| AC | ** | ** | * | * |
| AD | — | — | * | NT |
| AE | *** | *** | * | * |
| AF | * | * | * | NT |
| AG | * | ** | * | * |
| AH | ** | ** | * | * |
| AI | * | ** | * | * |
| AJ | ** | ** | * | * |
| AK | — | — | * | NT |
| AL | *** | *** | ** | * |
| AM | ** | ** | * | * |
| AN | *** | *** | * | * |
| AO | ** | *** | * | * |
| AP | *** | *** | * | * |
| AQ | *** | *** | * | * |
| AR | — | — | * | NT |
| AS | * | ** | * | * |
| AT | *** | *** | * | * |
| AU | — | — | * | NT |
| AV | *** | *** | * | * |
| AW | *** | ** | ** | * |
| AX | — | — | * | NT |
| AY | — | — | * | NT |
| AZ | ** | ** | * | * |
| BA | ** | *** | * | * |
| BB | ** | * | * | NT |
| BC | *** | *** | ** | ** |
| BD | * | * | * | NT |
| BE | *** | *** | * | * |
| BF | — | *** | * | NT |
| BG | * | ** | * | ** |
| BH | ** | ** | * | * |
| BI | * | *** | ** | ** |
| BJ | ** | *** | ** | ** |
| BK | — | *** | ** | ** |
| BL | * | *** | ** | ** |
| BM | ** | ** | * | * |
| BN | * | ** | ** | ** |
| BO | * | * | NT | ** |
| BP | — | — | * | * |
| BQ | ** | *** | NT | ** |
| CE | *** | *** | NT | ** |
| CF | *** | *** | NT | * |
| CG | NT | * | NT | * |
| CH | * | ** | BT | * |
| CI | *** | *** | NT | ** |
| CJ | — | — | NT | ** |
| CK | ** | ** | NT | ** |
| CL | * | * | NT | * |
| CM | — | — | NT | * |
| CN | NT | — | NT | * |
| CO | ** | * | NT | ** |

### EQUIVALENTS

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

1. A method for reducing antibiotic resistance of a microbial cell, comprising contacting said cell with a transcription factor modulating compound of the formula (I), (II), (III), (IV), (V), (VI), (VII) or (VIII): 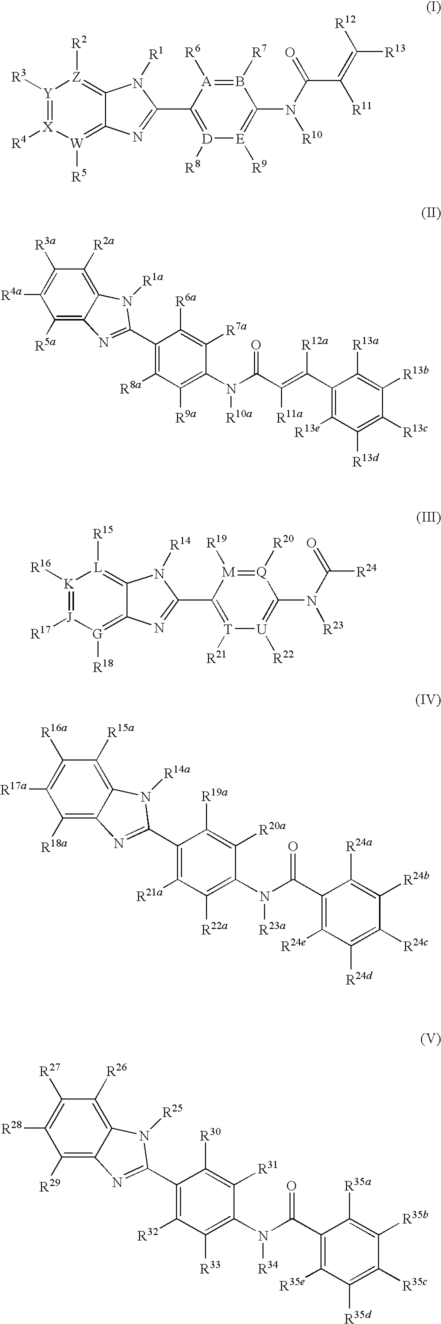 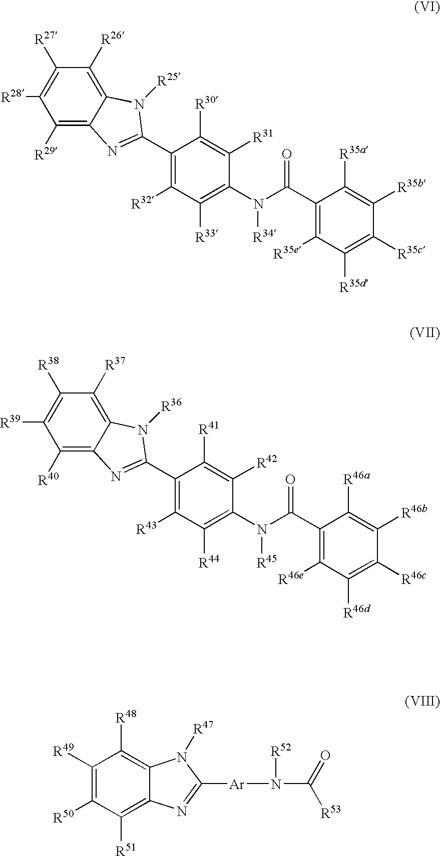
wherein
R¹, R^{1a}, R¹⁴, R^{14a}, R²⁵ are each independently hydroxyl, OCOCO₂H; a straight or branched C₁-C₅ alkyloxy group; or a straight or branched C₁-C₅ alkyl group;
A, B, D, E, G, J, K, L, M, Q, T, U, W, X, Y and Z are each independently carbon or nitrogen;
R², R³, R⁴, R⁵, R⁶, R⁷, R⁸, R⁹ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen when A, B, D, E, W, X, Y and Z are carbon; or R², R³, R⁴, R⁵, R⁶, R⁷, R⁸, R⁹ are each independently absent or hydroxyl when A, B, D, E, W, X, Y and Z are nitrogen;
R^{2a}, R^{3a}, R^{4a}, R^{5a}, R^{6a}, R^{7a}, R^{8a}, R^{9a}, R^{10a}, R^{11a}, R^{12a}, R^{13a}, R^{13b}, R^{13c}, R^{13d} and R^{13e} are each independently hydrogen, alkyl, alkenyl, alkynyl aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
R¹⁰, R¹¹, R¹² and R¹³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen;
R¹⁵, R¹⁶, R¹⁷, R¹⁸, R¹⁹, R²⁰, R²¹, R²², R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen, when G, J, K, L, M, Q, T and U are carbon; or R¹⁵, R¹⁶, R¹⁷, R¹⁸, R¹⁹, R²⁰, R²¹, R²², R²³ and R²⁴ are each independently absent or hydroxyl when G, J, K, L, M, Q, T and U are nitrogen;
R^{15a}, R^{16a}, R^{17a}, R^{18a}, R^{19a}, R^{20a}, R^{21a}, R^{22a}, R^{23a} and R^{24a}, R^{24b}, R^{24c}, R^{24d} and R^{24e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime,
R^{25′} is a substituted straight or branched C₁-C₅ alkyloxy group;
R²⁶, R²⁷, R²⁸, R²⁹, R³⁰, R³¹, R³², R³³, R³⁴, R^{35a}, R^{35b}, R^{35c}, R^{35d} and R^{35e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime amino-oxime, or halogen;
R^{26′}, R^{27′}, R^{28′}, R^{29′}, R^{30′}, R^{31′}, R^{32′}, R^{33′}, R^{34′}, R^{35a′}, R^{35b′}, R^{35c′}, R^{35d′} and R^{35e′} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino alkylamino, arylamino, CO₂H, cyano, nitro, CONH₂, heteroarylamino, oxime, alkyloxime, aryloxime, amino-oxime, or halogen;
R³⁶ is hydroxyl;
R³⁷, R³⁹, R⁴⁰, R⁴¹, R⁴², R⁴³, R⁴⁴, R⁴⁵, R^{46a}, R^{46b}, R^{46d}, and R^{46e} are each independently hydrogen, alkyl alkenyl, alkynyl, aryl, heteroaryl, heterocyclyl, alkoxy, aryloxy, heteroaryloxy, alkoxycarbonyl aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
R³⁸ is cyano, nitro, oxime, alkyloxime, aryloxime, heteroaryl, amino-oxime, or aminocarbonyl;
R^{46c} is hydrogen, acyl, fluoro, pyrizinyl, pyridinyl, cyano, imidazolyl, dialkylaminocarbonyl or dialkylamino;
R⁴⁷ is hydroxyl, OCOCO₂H, a straight or branched C₁-C₅ alkyloxy group, or a straight or branched C₁-C₅ alkyl group;
R⁴⁸, R⁴⁹, R⁵⁰, R⁵¹, R⁵² and R⁵³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
Ar is aryl;
provided that when A, B, C, D, E, W, X, Y and Z are each carbon, one of R⁶, R⁷, R⁸, R⁹ is not hydrogen,
provided that when R^{1a} is hydroxy R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b}, R^{13d}, and R^{13e} are hydrogen, then R^{13c} is not hydrogen, fluorine, dimethylamino, cyano, hydroxy, methyl or methoxy;
provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b} and R^{13d} are hydrogen, then R^{13c} and R^{13e} are not fluorine;
provided that when G, J, K, L, M, Q, T and U are each carbon, one of R¹⁵, R¹⁶, R¹⁷, R¹⁸, R¹⁹, R²⁰, R²¹, R²², R²³ and R²⁴, are not hydrogen;
provided that at least two of R^{24a}, R^{24b}, R^{24c}, R^{24d}, R²⁶, R²⁷, R²⁸ and R²⁹ are not hydrogen;
provided that when R³⁸ is nitro and R³⁷, R⁹¹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino, acyl or hydrogen; and
provided that when R³⁸ is cyano and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino; and pharmaceutically acceptable salts, esters and prodrugs thereof; such that the antibiotic resistance of said microbial cell is reduced.

2. A method for modulating transcription, comprising contacting a transcription factor with a transcription factor modulating compound of the formula (I), (II), (III), (IV), (V), (VI), (VII) or (VII): 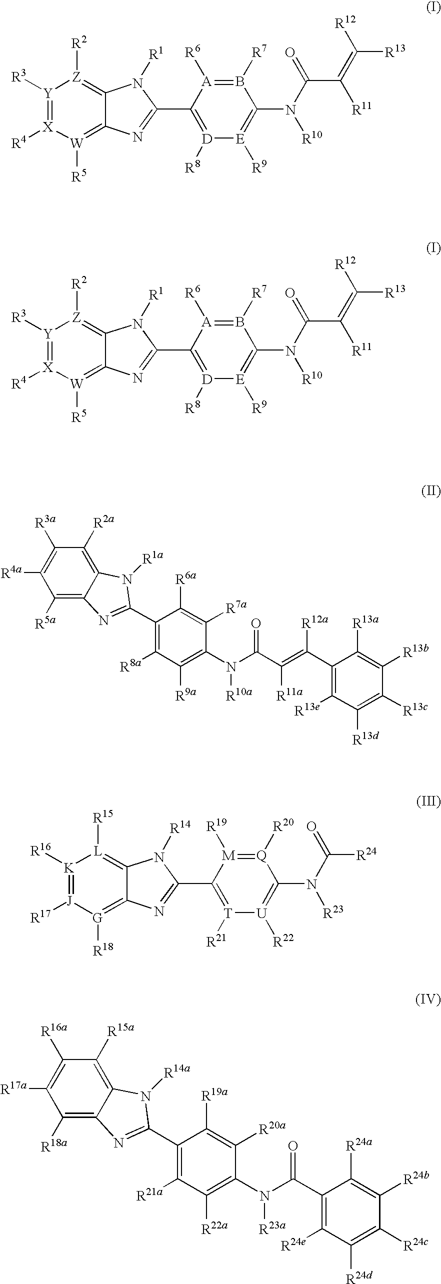 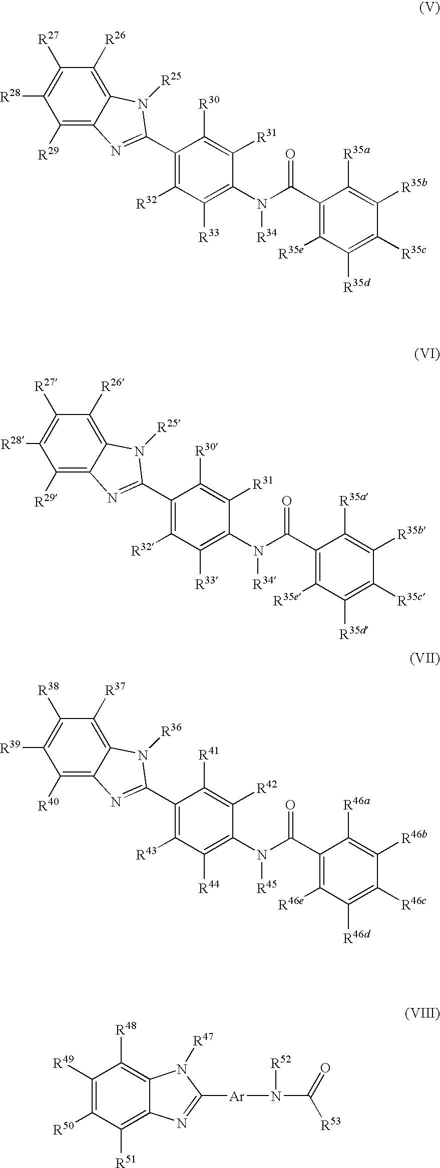
wherein
R¹, R^{1a}, R¹⁴, R^{14a}, R²⁵ are each independently hydroxyl, OCOCO₂H; a straight or branched C₁-C₅ alkyloxy group; or a straight or branched C₁-C₅ alkyl group;
A, B, D, E, G, J, K, L, M, Q, T, U, W, X, Y and Z are each independently carbon or nitrogen;
R², R³, R⁴, R⁵, R⁶, R⁷, R⁸, R⁹ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen when A, B, D, E, W, X, Y and Z are carbon; or R², R³, R⁴, R⁵, R⁶, R⁷, R⁸, R⁹ are each independently absent or hydroxyl when A, B, D, E, W, X, Y and Z are nitrogen;
R^{2a}, R^{3a}, R^{4a}, R^{5a}, R^{6a}, R^{7a}, R^{8a}, R^{9a}, R^{10a}, R^{11a}, R^{12a}, R^{13a}, R^{13b}, R^{13c}, R^{13d} and R^{13e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
R¹⁰, R¹¹, R¹² and R¹³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen;
R¹⁵, R¹⁶, R¹⁷, R¹⁸, R¹⁹, R²⁰, R²¹, R²², R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro, CONH₂, heteroarylamino, oxime, alkyloxime, aryloxime, amino-oxime, or halogen, when G, J, K, L, M, Q, T and U are carbon; or R¹⁵, R¹⁶, R¹⁷, R¹⁸, R¹⁹, R²⁰, R²¹, R²², R²³ and R²⁴ are each independently absent or hydroxyl when G, J, K, L, M, Q, T and U are nitrogen;
R^{15a}, R^{16a}, R^{17a}, R^{18a}, R^{19a}, R^{20a}, R^{21a}, R^{22a}, R^{23a} and R^{24a}, R^{24b}, R^{24c}, R^{24d} and R^{24e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime,
R^{25′} is a substituted straight or branched C₁-C₅ alkyloxy group;
R²⁶, R²⁷, R²⁸, R²⁹, R³⁰, R³¹, R³², R³³, R³⁴, R^{35a}, R^{35b}, R^{35c}, R^{35d} and R^{35e} are each independently hydrogen, alkyl, alkenyl, alkynyl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
R^{26′}, R^{27′}, R^{28′}, R^{29′}, R^{30′}, R^{31′}, R^{32′}, R^{33′}, R^{34′}, R^{35a′}, R^{35b′}, R^{35c′}, R^{35d′} and R^{35e′} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
R³⁶ is hydroxyl;
R³⁷, R³⁹, R⁴⁰, R⁴¹, R⁴², R⁴³, R⁴⁴, R⁴⁵, R^{46a}, R^{46b}, R^{46d} and R^{46e} are each independently hydrogen, alkyl alkenyl, alkynyl, aryl, heteroaryl, heterocyclyl, alkoxy, aryloxy, heteroaryloxy, alkoxycarbonyl aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
R³⁸ is cyano, nitro, oxime, alkyloxime, aryloxime, heteroaryl, amino-oxime, or aminocarbonyl;
R^{46c} is hydrogen, acyl, fluoro, pyrizinyl, pyridinyl, cyano, imidazolyl, dialkylaminocarbonyl or dialkylamino;
R⁴⁷ is hydroxyl. OCOCO₂H, a straight or branched C₁-C₅ alkyloxy group, or a straight or branched C₁-C₅ alkyl group;
R⁴⁸, R⁴⁹, R⁵⁰, R⁵¹, R⁵² and R⁵³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
Ar is aryl;
provided that when A, B, C, D, E, W, X, Y and Z are each carbon, one of R⁶, R⁷, R⁸, R⁹ is not hydrogen,
provided that when R^{1a} is hydroxy R^{3a} is nitro R^{2a}, R^{4a}, R^{5a}, R^{6a}, R^{7a}, R^{8a}, R^{9a}, R^{10a}, R^{11a}, R^{12a}, R^{13a}, R^{13b}, R^{13d}, and R^{13e} are hydrogen, then R^{13c} is not hydrogen, fluorine, dimethylamino, cyano, hydroxy, methyl or methoxy;
provided that when R^{1a} is hydroxyl R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b} and R^{13d} are hydrogen, then R^{13c} and R^{13e} are not fluorine;
provided that when G, J, K, L, M, Q, T and U are each carbon, one of R¹⁵, R¹⁶, R¹⁷, R¹⁸, R¹⁹, R²⁰, R²¹, R²², R²³ and R²⁴, are not hydrogen;
provided that at least two of R^{24a}, R^{24b}, R^{24c}, R^{24d}, R^{24e}, R²⁶, R²⁷, R²⁸ and R²⁹ are not hydrogen;
provided that when R³⁸ is nitro and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino, acyl or hydrogen; and
provided that when R³⁸ is cyano and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino; and pharmaceutically acceptable salts, esters and prodrugs thereof; such that transcription is modulated.

3.-253. (canceled)

254. A pharmaceutical composition comprising a pharmaceutically acceptable carrier and a transcription factor modulating compound, wherein said compound is of the formula (I), (II), (III), (IV), (V), (VI), (XVII), (XIII) or of Table 2.

255. The pharmaceutical composition of claim 254, further comprising an antibiotic.

256. The pharmaceutical composition of claim 254, wherein said effective amount is effective to treat a biofilm associated state in said subject.

257. The pharmaceutical composition of claim 256, wherein said biofilm associated state is selected from the group consisting of middle ear infections, cystic fibrosis, osteomyelitis, acne, dental cavities, endocarditis, and prostatitis.

258. A method for cleaning and disinfecting contact lenses comprising administering a composition comprising an acceptable carrier and a transcription factor modulating compound of the formula (I), (II), (III), (IV), (V), (VI), (VII), (VIII) or of Table 2.

259. A method of treating medical indwelling devices comprising administering a composition comprising a transcription factor modulating compound of the formula (I), (II), (III), (IV), (V), (VI), (VII), (VIII) or of Table 2.

260. The method of claim 259, wherein said device is selected from the group consisting of catheters, orthopedic devices and implants.

261. A method for treating or preventing a biofilm associated state in a subject, comprising administering to said subject an effective amount of a transcription factor modulating compound of the formula (I), (II), (III), (IV), (V), (VI), (VII), (VIII) or of Table 2.

262. The method of claim 261, wherein said biofilm associated state is selected from the group consisting of middle ear infections, cystic fibrosis, osteomyelitis, acne, dental cavities, endocarditis, and prostatitis.

263. The method of claim 261, further comprising administering a pharmaceutically acceptable carrier.

264. The method of claim 261, wherein said subject is a mammal.

265. The method of claim 264, wherein said subject is a human.

266. The method of claim 261, wherein said subject is immunocompromised.

267. A method for preventing a bacterial associated state in a subject, comprising administering to said subject an effective amount of a transcription factor modulating compound of the formula (I), (II), (III), (IV), (V), (VI), (VII), (VII) or of Table 2.

268. The method of claim 267, wherein said subject is a human.

269. The method of claim 267, wherein said transcription factor modulating compound is a MarA family polypeptide inhibitor.

270. The method of claim 267, wherein said transcription factor modulating compound is an AraC family polypeptide inhibitor.

271. A method for treatment of a urinary tract infection in a subject, comprising administering to said subject an effective amount of a compound of the formula (I), (II), (III), (IV), (V), (VI), (VII), (VIII) or of Table 2.

272. The method of claim 271, wherein the subject is a human or a cat.

273. A compound of formula (I), (II), (III), (IV), (V), (VI), (VII) or (VIII): 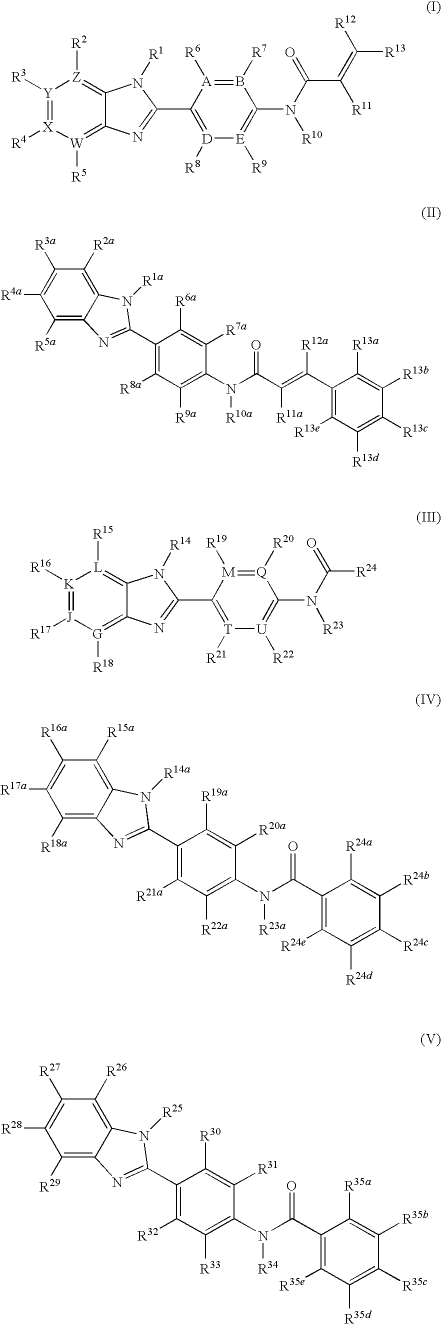 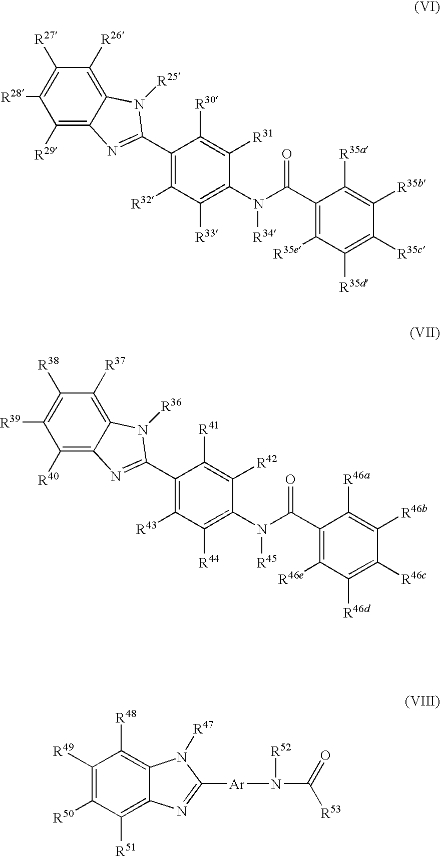
wherein
R¹, R^{1a}, R¹⁴, R^{14a}, R²⁵ are each independently hydroxyl, OCOCO₂H; a straight or branched C₁-C₅ alkyloxy group; or a straight or branched C₁-C₅ alkyl group;
A, B, D, E, G, J, K, L, M, Q, T, U, W, X, Y and Z are each independently carbon or nitrogen;
R², R³, R⁴, R⁵, R⁶, R⁷, R⁸, R⁹ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen when A, B, D, E, W, X, Y and Z are carbon; or R², R³, R⁴, R⁵, R⁶, R⁷, R⁸, R⁹ are each independently absent or hydroxyl when A, B, D, E, W, X, Y and Z are nitrogen;
R^{2a}, R^{3a}, R^{4a}, R^{5a}, R^{6a}, R^{7a}, R^{8a}, R^{9a}, R^{10a}, R^{11a}, R^{12a}, R^{13a}, R^{13b}, R^{13d} and R^{13e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
R¹⁰, R¹¹, R¹² and R¹³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime or halogen;
R¹⁵, R¹⁶, R¹⁷, R¹⁸, R¹⁹, R²⁰, R²¹, R²², R²³ and R²⁴ are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl alkylcarbonyl, arylcarbonyl, heteroarylcarbonyl, acyl, acylamino, amino, alkylamino, arylamino, absent, CO₂H, cyano, nitro CONH₂, heteroarylamino, oxime, alkyloxime, aryloxime, amino-oxime, or halogen, when G, J, K, L, M, Q, T and U are carbon; or R¹⁵, R¹⁶, R¹⁷, R¹⁸, R¹⁹, R²⁰, R²¹, R²², R²³ and R²⁴ are each independently absent or hydroxyl when G, J, K, L, M, Q, T and U are nitrogen;
R^{15a}, R^{16a}, R^{17a}, R^{18a}, R^{19a}, R^{20a}, R^{21a}, R^{22a}, R^{23a} and R^{24a}, R^{24b}, R^{24c}, R^{24d} and R^{24e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime.
R^{25′} is a substituted straight or branched C₁-C₅ alkyloxy group;
R²⁶, R²⁷, R²⁸, R²⁹, R³⁰, R³¹, R³², R³³, R³⁴, R^{35a}, R^{35b}, R^{35c}, R^{35d} and R^{35e} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
R^{26′}, R^{27′}, R^{28′}, R^{29′}, R^{30′}, R^{31′}, R^{32′}, R^{33′}, R^{34′}, R^{35a′}, R^{35b′}, R^{35c′}, R^{35d′}, and R^{35e′} are each independently hydrogen, alkyl, alkenyl, alkynyl, aryl, heteroaryl, heterocyclyl, alkoxy, aryloxy, heteroaryloxy, alkoxycarbonyl, aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino alkylamino, arylamino, CO₂H, cyano, nitro, CONH₂, heteroarylamino, oxime, alkyloxime, aryloxime, amino-oxime, or halogen;
R³⁶ is hydroxyl;
R³⁷, R³⁹, R⁴⁰, R⁴¹, R⁴², R⁴³, R⁴⁴, R⁴⁵, R^{46a}, R^{46b}, R^{46d}, and R^{46e} are each independently hydrogen, alkyl alkenyl, alkynyl, aryl, heteroaryl, heterocyclyl, alkoxy, aryloxy, heteroaryloxy, alkoxycarbonyl aryloxycarbonyl, heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
R³⁸ is cyano, nitro, oxime, alkyloxime, aryloxime, heteroaryl, amino-oxime, or aminocarbonyl;
R^{46c} is hydrogen, acyl, fluoro, pyrizinyl, pyridinyl, cyano, imidazolyl, dialkylaminocarbonyl or dialkylamino;
R⁴⁷ is hydroxyl, OCOCO₂H, a straight or branched C₁-C₅ alkyloxy group, or a straight or branched C₁-C₅ alkyl group;
R⁴⁸, R⁴⁹, R⁵⁰, R⁵¹, R⁵² and R⁵³ are each independently hydrogen, alkyl, alkenyl, alkynyl, aryl, heteroaryl, heterocyclyl, alkoxy, aryloxy, heteroaryloxy, alkoxycarbonyl, aryloxycarbonyl heteroaryloxycarbonyl, alkylsulfonyl, arylsulfonyl, aminosulfonyl, alkylcarbonyl, arylcarbonyl, heteroarylcarbonyl, acyl, acylamino, amino, alkylamino, arylamino, CO₂H, cyano, nitro, CONH₂, heteroarylamino, oxime, alkyloxime, aryloxime, amino-oxime, or halogen;
Ar is aryl;
provided that when A, B, C, D, E, W, X, Y and Z are each carbon, one of R⁶, R⁷, R⁸, R⁹ is not hydrogen,
provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b}, R^{13d}, and R^{13e} are hydrogen, then R^{13c} is not hydrogen, fluorine, dimethylamino, cyano, hydroxy, methyl or methoxy;
provided that when R^{1a} is hydroxy, R^{3a} is nitro, R^{2a}, R^{4a}, R^{5a}, R^{6a}, R^{7a}, R^{8a}, R^{9a}, R^{10a}, R^{11a}, R^{12a}, R^{13a}, R^{13b} and R^{13d} are hydrogen, then R^{13c} and R^{13e} are not fluorine;
provided that when G, J, K, L, M, Q, T and U are each carbon, one of R¹⁵, R¹⁶, R¹⁷, R¹⁸, R¹⁹, R²⁰, R²¹, R²², R²³ and R²⁴, are not hydrogen;
provided that at least two of R^{24a}, R^{24b}, R^{24c}, R^{24d}, R^{24e}, R²⁶, R²⁷, R²⁸ and R²⁹ are not hydrogen;
provided that when R³⁸ is nitro and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino, acyl or hydrogen; and
provided that when R³⁸ is cyano and R³⁷, R³⁹, R⁴⁰, R⁴¹, R⁴², R⁴³, R⁴⁴, R⁴⁵, R^{46a}, R^{46b}, R^{46d}, and R^{46e} are each hydrogen, then R^{46c} is not dialkylamino; and pharmaceutically acceptable salts, esters and prodrugs thereof.

274.-280. (canceled)

281. A compound of claim 273, wherein said compound is a compound of Table 2.

282. The compound of claim 273, wherein said pharmaceutically acceptable salt is a sodium salt or a potassium salt.


SEQUENCE LISTING (7 sequences)

SEQ ID NO: 1 - protein, 20 aa, Unknown
  Description of Unknown MarA family peptide
XXXXAXXXXXSXXXLXXXFX

SEQ ID NO: 2 - protein, 22 aa, Unknown
  Description of Unknown MarA family peptide
XXIXXIAXXXGFXSXXXFXXXX

SEQ ID NO: 3 - protein, 22 aa, Unknown
  Description of Unknown MarA family peptide
XXXAXXXGXSXXXLQXXFXXXX

SEQ ID NO: 4 - protein, 23 aa, Unknown
  Description of Unknown MarA family peptide
IXDIAXXXGFXSXXFXXXFXXXX

SEQ ID NO: 5 - protein, 22 aa, Unknown
  Description of Unknown MarA family peptide
EKVSERSGYSKWHLQRMFKKET

SEQ ID NO: 6 - protein, 24 aa, Unknown
  Description of Unknown MarA family peptide
ILYLAERYGFESQQTLTRTFKNYF

SEQ ID NO: 7 - protein, 6 aa, Artificial Sequence
  Description of Artificial Sequence Synthetic 6xHis tag
HHHHHH